(19) United States
(12) Patent Application Publication
(10) Pub. No.: US 2005/0089858 A1
(43) Pub. Date: Apr. 28, 2005

(54) Salvador tumor suppressor gene

(76) Inventors: Iswar K. Hariharan, Berkeley, CA (US); Nicholas A.M. Tapon, Somervile, MA (US); Daniel A. Haber, Newton, MA (US); Daphne W. Bell, Arlington, MA (US); Doke C.R. Wahrer, Boston, MA (US); Kiernan Harvey, Berkeley, CA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/493,759

(22) PCT Filed: Oct. 25, 2002
(86) PCT No.: PCT/US02/34354

Related U.S. Application Data
(60) Provisional application No. 60/348,432, filed on Oct. 26, 2001.

(51) Int. Cl. A61K 48/00; C12Q 1/68

(52) U.S. Cl. 435/6; 514/44

(57) ABSTRACT

The invention relates to the discovery of a novel Drosophila gene, salavador, and to the discovery of a tumor suppressor function for its human counterpart. The salvador nucleic acid and protein molecules, their use in the diagnosis and treatment of disorders characterized by aberrant salvador molecule expression, as well as various research uses are described.

### RELATED APPLICATIONS

This application claims priority to U.S. Application Ser. No. 60/348,432, filed Oct. 26, 2001, which is hereby incorporated by reference.

### GOVERNMENT SUPPORT

This invention was made in part with support from the National Institutes of Health, a United States Government Agency, under grant numbers EY11632, GM61672, and CA87691. The Government may have certain rights in this invention.

### FILED OF THE INVENTION

This invention relates to the discovery of a novel Drosophila gene, Salvador, and to the discovery of a tumor suppressor function for its human counterpart. The invention is directed to the isolated tumor suppressor nucleic acids, the proteins encoded by these nucleic acids, binding agents that selectively bind thereto, and various diagnostic, therapeutic and research uses of these compositions.

### BACKGROUND OF THE INVENTION

The number of cells in an organism is determined by the number of cells generated as a result of cell proliferation as well the number of cells that are eliminated by cell death. Both cell proliferation and cell death are strictly regulated by developmental mechanisms to ensure that an organ of a characteristic shape and size is generated. The very mechanisms that regulate normal growth and cell proliferation are often those that are perturbed in human cancers. Mutational events found in cancers can either promote growth and cell proiferation or impede cell death.

Cancer progression is caused by accumulation of multiple mutations that provide selective advantage during cancer growth, invasion and metastasis (Haber, D. A. and Fearon, E. R. Lancet 351 Suppl 2:SII1-8, 1998; Loeb, K. R. and Loeb, L. A., Carcinogenesis 21(3):379-85, 2000; Loeb, L. A., et al. Cancer Research 34 (9):2311-21, 1974). While gain of function mutations occur in oncogenes, many of the genetic events that underlie cancer appear to be inactivating, or loss of function mutations, affecting tumor suppressor genes (Haber, D. A. and Fearon, E. R. Lancet 351 Suppl 2:SII1-8, 1998). Tumor suppressor genes identified to date exhibit diverse cellular functions (Haber D. and Harlow E. Nature Genetics 16 (4):320-2, 1997).

Functional studies on these tumor suppressor genes have supported the original hypothesis that these genes represent potential bottlenecks in wide variety of cellular pathways, including proliferation, differentiation, apoptosis and response to DNA damage (Haber D. and Harlow E. Nature Genetics 16 (4):320-2, 1997). For example, p53 and WT1 are DNA binding transcription factors; RB1, APC and possibly BRCA1 indirectly modulate transcription; P16 is an inhibitor of kinases required for cell cycle progression; PTEN is a novel phosphatase; NF2 is a cell structural component; and VHL is a potential mediator of mRNA processing. A large number of genes are believed to be genomic caretakers and mutations in these genes cause microsatellite instability (e.g., MSH2, MLH1, PMS1 and PMS2) or chromosomal instability (e.g., p53, possibly BRCA1 and BRCA2). To date, genes involved in advanced stages of cancer progression such as invasion, angiogenesis and metastasis have not been identified (Haber D. and Harlow E. Nature Genetics 16 (4):320-2, 1997; Fearon, E. R. Current Biology 9 (23):R873-5, 1999). They are likely to become evident over time with large-scale genome wide analysis.

The vast majority of cancers result from sporadic genetic events and only rare cases (fewer than 1%) have an inherited component (Fearon, E. R. Science 278 :1043-50, 1997; Kinzler, K. W. and Vogelstein B. Cell. 87 (2):159-70, 1996). However, the isolation of tumor suppressor genes has typically originated from genetic analysis of such rare inherited cancer syndromes (Fearon, E. R. Science 278 :1043-50, 1997). Linkage analysis on large families with cancer present in multiple generations allows identification of markers that co-segregate with cancer. In some cases cytogenetic abnormalities could also be observed either in sporadic or in germline tumors (Fearon, E. R. Science 278 :1043-50, 1997; Gray, J. W. and Collins, C. Carcinogenesis. 21 (3):443-52, 2000). For example, a small fraction of retinoblastomas have a homozygous deletion of RB 1 gene (Fearon, E. R. Science 278 :1043-50, 1997). Rare Wilms tumor and colon cancer have deletions of WT1 and APC, respectively. These germline or sporadic homozygous deletions have been instrumental in tumor suppressor gene cloning efforts (Fearon, E. R. Science 278 :1043-50, 1997). Allelic losses in tumors are typically detected as “loss of heterozygosity” or “LOH.” This represents loss of a polymorphic marker, commonly resulting from a large interstitial deletion or chromosomal non-disjunction event. While LOH is a common event in cancer, it only allows rough mapping of tumor suppressor loci (Gray, J. W. and Collins, C. Carcinogenesis. 21 (3):443-52, 2000). The large size of the LOH region (>10 Mb) makes the identification of the specific tumor suppressor gene targeted by mutation difficult. In contrast, homozygous deletions in tumors are typically small (<100 Kb) since they are restricted by the deletion of the flanking genes. Homozygous deletions occur by diverse mechanisms, including a small deletion in one allele accompanied by LOH of the second allele, or even large deletion of each allele whose common region of overlap is small. Identification of such homozygous deletions can be a powerful approach to identify tumor suppressor genes (Fearon, E. R. Science 278:1043-50, 1997).

Significant technological advances have been made to identify regions of chromosomes involved in tumor progression. Analyses of metaphase chromosomes show chromosomal rearrangements in leukemia and lymphomas (Rowley, J. D. Annu. Rev. Genet. 32 :495-519). This is more difficult in solid tumors where karyotyping is less commonly performed. Fluorescence in situ hybridization (FISH) has greatly improved the sensitivity and specificity of detecting chromosome aberrations (Pinkel, D., et al. Proc. Natl. Acad. Sci. 85 9138-9142, 1988; Speicher, M. R. and Ward D. C. Nature Med. 2:1046-1048 1996). However, its application in human malignancies is still limited because of the complex karyotypes seen in clinical samples. Comparative genome hybridization (CGH) uses both normal and tumor genomes to identify regions in tumor DNA that have undergone changes in copy number (Kallioniemi, O. P., et al. Seminars in Cancer Biology. 4 (1):41-6, 1993). In this technique, normal and tumor DNA are labeled with two different haptens that fluoresce at different wavelengths. The probes are then hybridized to metaphase chromosomes in the presence of excess Cot-i DNA thus inhibiting hybridization of labeled repetitive sequences. The ratio of the amount of two genomes that hybridize to specific areas of the chromosomes indicates the copy number of the two samples. CGH is currently limited to a resolution of 10 to 20 Mb and more sensitive in detecting amplifications rather than a small deletion (Gray, J. W. and Collins, C. Carcinogenesis. 21 (3):443-52, 2000). An alternative method, Representational Difference Analysis (RDA) is a PCR based subtractive hybridization technique, that is particularly applicable in isolating homozygous deletions in tumors (Lisitsyn, N., et al. Science. 259 (5097):946-51, 1993; Lisitsyn N A. Trends in Genetics 11 (8):303-7, 1995; Lisitsyn, N. and Wigler, M. Methods in Enzymology. 254:291-304, 1995). It has already been successful in isolating tumor suppressor genes PTEN and DMBT1 and has played a significant role in cloning of BRCA2 (Mollenhauer, J., et al. Nature Genetics. 17 (1):32-9, 1997; Li, J., et al. Science 275, 1943-1947, 1997; Schutte, M., et al. Proc. Natl. Acad Sci. 92, 5950-5954, 1995).

The Drosophila compound eye is particularly suited to the application of genetic approaches to the study of cell proliferation and cell death in the context of organ development (Wolff, T. and Ready, D. F. (1993) Pattern formation in the Drosophila retina. In The Development of Drosophila melanogaster, M. Bate, and A. Martinez Arias, eds. (Plainview, N.Y.: Cold Spring Harbor Laboratory Press) 1277-1325). The adult eye develops from a primordium consisting of approximately 30 cells in the embryo. Cell growth and proliferation occur during all stages of larval development. Most of the cells generated lo adopt specialized fates (e.g., photoreceptor, pigment cell) during the late larval and pupal stages, leaving approximately 2000 unspecified cells. These excess cells are subsequently eliminated by a wave of apoptosis. Thus, the final number of cells in the adult eye can be altered by changes in either cell proliferation or cell death.

While the developmental signals that trigger cell cycle exit or apoptosis in Drosophila are still poorly characterized, considerable progress has been made in identifying the endpoints of these pathways. In many different tissues, cell cycle exit appears to be contingent on the downregulation of Cyclin E levels (Knoblich, J. A., et al. (1994) Cell 77, 107-120). This coincides with increased expression of the cdk inhibitor Dacapo during the final cell cycle (de Nooij, J. C., et al. (1996) Cell 87, 1237-1247; Lane, M. E., et al. (1996) Cell 87, 1225-1235). Dacapo inactivates residual Cyclin E/cdk2 complexes and facilitates a precisely timed exit from the cell cycle. The decrease in Cyclin E Summary levels is primarily achieved by a reduction in its transcription, but other mechanisms including degradation of Cyclin E protein appear to be important (Jones, L., et al. (2000) Development 127, 46194630; Moberg, K. H., et al. (2001) Nature 413, 311-316). Developmentally regulated cell death in the pupal retina is mediated by caspase activation. The Reaper, Hid, and Grim proteins bind to the Drosophila inhibitor of apoptosis 1 (DIAP1) protein and prevent DIAP1 from inhibiting caspases (Goyal, L., et al. (2000) EMBO J 19, 589-597; Lisi, S., et al. (2000) Genetics 154, 669-678; Wang, S. L. (1999) Cell 98, 453-463).

### SUMMARY OF THE INVENTION

The invention is based, in part, on the discovery of a novel Drosophila gene, termed salvador, which is mutated in tumorous outgrowths (Genbank Accession Number AY131211). Three mutations were identified in the salvador locus that give mutant Drosophila cells a proliferative advantage with respect to wild type cells. The Drosophila salvador gene was cloned and found to encode a novel protein having two WW domains with highly conserved orthologues in C. elegans and mammals. The three salvador mutations identified in Drosophila result in proteins lacking both WW domains.

The human homologue of salvador, hWW45 (GenBank accession number NM0218118), has been identified, but its function was previously unknown (Valverde, P. Biochem. Biophys. Res. Commun. 276: 990-998, 2000). The Drosophila and human salvador gene products share 47% identity and 54% similarity over the C-terminal 188 amino acids. The invention also is based, in part, on the discovery that the human counterpart of the Drosophila gene is deleted or mutated in at least three tumor-derived cancer cell lines. Accordingly, although not wishing to be bound to any particular theory or mechanism, it is believed that the salvador genes from various species play a role in modulating cellular development (including cell proliferation, growth, and death) by regulating both cell cycle exit and apoptosis. Thus, the invention is directed to novel compositions of Salvador human and Drosophila nucleic acids and proteins encoded thereby (SEQ ID NOs: 1, 2, 3, and 4), as well as to agents that selectively bind to these novel molecules. Further, the invention includes diagnostic, therapeutic, and research applications of these compositions.

According to one aspect of the invention, an isolated nucleic acid molecule is provided. The isolated nucleic acid molecule is selected from:
(a) nucleic acid molecules which hybridize under stringent conditions to a nucleic acid molecule having a nucleotide sequence set forth as SEQ ID NO:1 (human salvador cDNA) or SEQ ID NO:3 (Drosophila salvador cDNA), and which code for a Salvador protein,
(b) deletions, additions and substitutions of the nucleic acid molecules of (a), which code for a Salvador protein,
(c) nucleic acid molecules that differ from the nucleic acid molecules of (a) or (b) in codon sequence due to the degeneracy of the genetic code, or
(d) complements of (a), (b) or (c).

The preferred isolated nucleic acids of the invention are salvador nucleic acid molecules which encode a Salvador protein and include nucleic acid molecules that encode both wild-type and mutant Salvador proteins. As used herein, a Salvador protein refers to a protein which is encoded by a nucleic acid having SEQ ID NO:1 or SEQ ID NO:3; a functional fragment or equivalent thereof, provided that the functional fragment or equivalent encodes a protein which modulates cellular development by, for example, modulating cell maturation/differentiation, cell growth, cell proliferation, and/or cell death; wild-type Salvador proteins; and mutant Salvador proteins. Methods to determine whether a protein modulates cellular development involve introducing the protein (or the nucleic acid which encodes the protein) into the cell and observing a change in cellular development in accordance with standard procedures known to those of ordinary skill in the art. Such methods can be employed in vivo (e.g., observing tumor suppression) as well as in vitro (e.g., apoptosis assays). In a preferred embodiment, the isolated nucleic acid molecule is SEQ ID NO:1 or SEQ ID NO:3.

Other preferred isolated nucleic acids are fragments comprising one or more WW domains of a salvador gene or functional portions thereof.

According to another aspect of the invention, further isolated nucleic acid molecules that are based on the above-noted salvador nucleic acid molecules are provided. In this aspect, the isolated nucleic acid molecules are selected from: (a) a unique fragment of the nucleotide sequence set forth as SEQ ID NO:1 or set forth as SEQ ID NO:3 between 12 and Is 2000 nucleotides in length (more preferably, between 12 and 1400 nucleotides in length and, most preferably, between 12 and 32 nucleotides in length) or more and (b) complements of (a), wherein the unique fragments exclude nucleic acids having nucleotide sequences that are contained within SEQ ID NO:1 or SEQ ID NO:3, and that are known as of the priority date of this application. Known fragments, i.e., fragments that are not unique, of SEQ ID NO:1 and SEQ ID NO:3 are provided in Table 1 and identified by their positions in SEQ ID NO:1 and SEQ ID NO:3.

In a preferred embodiment, the unique fragments comprise the sequences selected from the group consisting of SEQ ID NO:7, SEQ ID NO:8, SEQ ID NO:9, SEQ ID NO:10, SEQ ID NO:11, SEQ ID NO:12, SEQ ID NO:13, and SEQ ID NO:14.

In yet another aspect of the invention, Mutant Salvador nucleic acid molecules are provided. The Mutant salvador nucleic acid molecules contain a sequence which is the same as SEQ ID NO:1 or SEQ ID NO:3, with the exception that the sequence includes one or more mutations, e.g., point mutations, deletion mutations, or truncations, such that the Mutant Salvador nucleic acid molecule does not encode a functional Salvador protein. Rather, the Mutant Salvador nucleic acid molecules encode a Mutant Salvador protein, i.e., a protein which does not exhibit Salvador protein functional activity. In certain preferred embodiments, the Mutant Salvador nucleic acid molecules are truncated forms of SEQ ID NO: 1 or SEQ ID NO:3 which lack one or more WW domains and/or one or more functional domains.

According to yet another aspect of the invention, isolated nucleic acid molecules are provided which are based upon the salvador gene genomic sequence (SEQ ID NO:6) and are useful, for example, for detecting the salvador nucleic acid molecules of the invention. According to this aspect of the invention, the isolated nucleic acid molecule is selected from:
(a) nucleic acid molecules which hybridize under stringent conditions to a nucleic acid molecule having a nucleotide sequence set forth as SEQ ID NO:6 (human salvador genomic DNA), and which codes for a Salvador protein,
(b) deletions, additions and substitutions of the nucleic acid molecules of (a), which code for a Salvador protein,
(c) nucleic acid molecules that differ from the nucleic acid molecules of (a) or (b) in codon sequence due to the degeneracy of the genetic code, and
(d) complements of (a), (b) or (c).

The preferred isolated nucleic acids of the invention are salvador nucleic acid molecules which encode a Salvador protein as defined above.

According to yet another aspect of the invention, unique fragments of the salvador gene genomic sequence are provided. In this aspect, the isolated nucleic acid molecules are selected from the group consisting of:
(a) a unique fragment of the nucleotide sequence set forth as SEQ ID NO:6 between 12 and 2000 nucleotides in length or more and
(b) complements of (a),
   wherein the unique fragments exclude nucleic acids having nucleotide sequences that are contained within SEQ ID NO:6, and that are known as of the priority date of this application. In preferred embodiments, the nucleic acid molecule is selected from the group consisting of: an intron of SEQ ID NO:6, a unique fragment of an intron of SEQ ID NO:6, and complements of the foregoing. In a particularly preferred embodiment, the isolated nucleic acid molecule comprises SEQ ID NO:5.

According to yet another aspect of the invention, an expression vector comprising any of the isolated nucleic acid molecules of the invention operably linked to a promoter is provided. In a related aspect, host cells transformed or transfected with such expression vectors also are provided.

According to still a further aspect of the invention, a transgenic non-human animal comprising an expression vector of the invention is provided. Also provided are transgenic non-human animals which have reduced or increased expression of a wild-type salvador nucleic acid molecule and/or reduced or increased expression of a Mutant salvador nucleic acid molecule. Increased and decreased expression levels are measured using methods known to those of skill in art, such as comparing an expression level of a gene or protein of interest in a sample versus a normal expression level, for example, the average expression level of several (e.g., 10) samples.

According to another aspect of the invention, an isolated polypeptide encoded by any of the foregoing isolated nucleic acid molecules of the invention is provided. Preferably, the isolated polypeptide comprises SEQ ID NO: 2 or SEQ ID NO: 4. In yet alternative embodiments, the isolated polypeptide comprises a truncated form of SEQ ID NO:2 or SEQ ID NO:4 which lacks one or more WW domains, lacks one or more functional WW domains, or otherwise includes modifications which result in a protein that does not exhibit a Salvador functional activity, i.e., a Mutant Salvador protein.

In yet a further aspect of the invention, binding polypeptides that selectively bind to a salvador molecule, including a wild-type and/or Mutant salvador molecule, are provided. According to this aspect, the binding polypeptides bind to an isolated nucleic acid or protein of the invention, including binding to unique fragments thereof. Preferably, the binding polypeptides bind to a Salvador protein, a Mutant Salvador protein, or a unique fragment of the foregoing. In certain particularly preferred embodiments, the binding polypeptide binds to a Mutant Salvador protein but does not bind to a Salvador protein, i.e., the binding polypeptides are selective for binding to the Mutant protein and can be used in various assays to detect the presence of the Mutant Salvador protein without detecting the wild type Salvador protein.

In preferred embodiments, the binding polypeptide is an antibody or antibody fragment, more preferably, an Fab or F(ab)₂ fragment of an antibody. Typically, the fragment includes a CDR3 region that is selective for the Salvador protein and/or Mutant Salvador protein. Any of the various types of antibodies can be used for this purpose, including monoclonal antibodies, humanized antibodies, and chimeric antibodies.

According to a further aspect of the invention, pharmaceutical compositions containing the nucleic acids, proteins, and binding polypeptides of the invention are provided. The pharmaceutical compositions contain any of the foregoing salvador molecules, Mutant salvador molecules, or binding agents in a pharmaceutically acceptable carrier. Thus, in a related aspect, the invention provides a method for forming a medicament that involves placing a therapeutically effective amount of the foregoing agent(s) in the pharmaceutically acceptable carrier to form one or more doses.

According to another aspect of the invention, various diagnostic methods are provided. In general, the methods are for diagnosing “a disorder characterized by aberrant expression of a salvador molecule.” As used herein, a “disorder characterized by aberrant expression of a salvador molecule” refers to a disorder in which there is a detectable difference in the expression levels of salvador molecule(s) including wild-type and/or Mutant salvador molecule(s) in cells that exhibit abnormal cellular development, compared to the expression levels of these molecule(s) in cells which do not exhibit such abnormal cellular development. Abnormal cellular development is determined in accordance with standard procedures known in the art to assess cell growth, cell maturation/differentiation, cell proliferation, cell-cell interactions, and cell death (including, e.g., apoptosis). Thus, the observation of normal cells is used to establish a control standard for normal cellular development in a particular cell type.

A disorder characterized by aberrant expression of a salvador molecule embraces a disorder characterized by underexpression (including no detectable expression) of a wild-type salvador nucleic acid molecule or a Salvador protein compared to control levels of these molecules (i.e., levels present in cells which do not exhibit abnormal cellular development, such as cells obtained from a subject who does not have a disorder characterized by aberrant cellular development), as well as overexpression of a Mutant salvador nucleic acid molecule or Mutant Salvador protein compared to control levels of these molecules. Such differences in expression levels can be determined in accordance with the diagnostic methods of the invention as disclosed herein. Exemplary categories of disorders that are characterized by aberrant expression of a salvador molecule include various cancers, birth defects, and autoimmunity disorders.

As noted above, “aberrant expression” refers to either or both of a decreased expression (including no detectable expression) of the salvador molecule (nucleic acid or protein) or an increased expression of a “Mutant salvador molecule.” A Mutant salvador molecule refers to a salvador nucleic acid molecule which includes a mutation (point mutation, addition, deletion, rearrangement, substitution, truncation, and the like) or to a Salvador protein molecule (e.g., gene product of a Mutant salvador nucleic acid molecule) which includes a mutation, provided that the mutation results in a Mutant Salvador protein that does not have the Salvador functional activity that is exhibited by a Salvador protein as described herein. The diagnostic methods of the invention can be used to detect the presence of a disorder associated with aberrant expression of a salvador molecule, as well as to assess the progression and/or regression of the disorder such as in response to treatment (e.g., chemotherapy, radiation). According to this aspect of the invention, the method for diagnosing a disorder characterized by aberrant expression of a salvador molecule involves: detecting in a first biological sample obtained from a subject, expression of a salvador molecule or a Mutant salvador molecule; wherein decreased expression of a salvador molecule or increased expression of a Mutant salvador molecule compared to a control sample indicates that the subject has a disorder characterized by aberrant expression of a salvador molecule.

In yet other embodiments, the diagnostic methods are useful for diagnosing the progression of a disorder. According to these embodiments, the methods further involve: detecting in a second biological sample obtained from the subject, expression of a salvador molecule or a Mutant salvador molecule, and comparing the expression of the salvador molecule or the Mutant salvador molecule in the first biological sample and the second biological sample. In these embodiments, a decrease in the expression of the salvador molecule in the second biological sample compared to the first biological sample or an increase in the expression of the Mutant salvador molecule in the second biological sample compared to the first biological sample indicates progression of the disorder.

In yet other embodiments, the diagnostic methods are useful for diagnosing the regression of a disorder. According to these embodiments, the methods further involve: detecting in a second biological sample obtained from the subject, expression of a salvador molecule or a Mutant salvador molecule, and comparing the expression of the salvador molecule or the Mutant salvador molecule in the first biological sample and the second biological sample. In these embodiments, an increase in the expression of the salvador molecule in the second biological sample compared to the first biological sample or a decrease in the expression of the Mutant salvador molecule in the second biological sample compared to the first biological sample indicates regression of the disorder.

In certain embodiments, the diagnostic methods of the invention involve detecting a salvador molecule that is a salvador nucleic acid molecule including a wild-type and/or Mutant salvador nucleic acid molecule as described above. In yet other embodiments, the methods involve detecting a Salvador protein or Mutant Salvador protein as described above. Various detection methods can be used to practice the diagnostic methods of the invention. For example, the methods can involve contacting the biological sample with an agent that selectively binds to the salvador molecule, to the salvador gene genomic sequence, or to the Mutant salvador molecule to detect these molecules. In certain embodiments, the salvador molecule is a nucleic acid and the method involves using an agent that selectively binds to the salvador molecule or to the Mutant Salvador molecule, e.g., a nucleic acid that hybridizes to SEQ ID NO:1 or to SEQ ID NO:3 under stringent conditions. Alternatively, the method involves using nucleic acids that hybridize under stringent conditions to a SEQ ID NO:6 that includes the coding sequence of the salvador gene. Such nucleic acid probes include nucleic acids which hybridize to intron portions of the salvador gene (e.g., SEQ ID NO:5). In yet other embodiments, the salvador molecule is a protein and the method involves using an agent that selectively binds to the Salvador protein or to the Mutant Salvador protein, e.g., a binding polypeptide, such as an antibody, that selectively binds to SEQ ID NO:2 or to SEQ ID NO:4.

According to still another aspect of the invention, kits for performing the diagnostic methods of the invention are provided. The kits are nucleic acid-based kits or protein-based kits. According to the former embodiment, the kits include one or more nucleic acid molecules that hybridize to a salvador nucleic acid molecule including to a wild-type or Mutant salvador nucleic acid molecule under stringent conditions; one or more control agents; and instructions for the use of the nucleic acid molecules, and agents in the diagnosis of a disorder associated with aberrant expression of a salvador molecule. Nucleic acid-based kits optionally further include a first primer and a second primer, wherein the first primer and the second primer are constructed and arranged to selectively amplify at least a portion of a salvador nucleic acid molecule comprising SEQ ID NO:1 or SEQ ID NO:6 or to selectively amplify a portion of a Mutant salvador nucleic acid molecule. Alternatively, the kits include two isolated nucleic acid molecules, the first consisting of a 20-32 nucleotide contiguous segment of SEQ ID NO:1 or SEQ ID NO:6 and the second consisting of a 20-32 nucleotide contiguous segment of the complement of SEQ ID NO:1 or SEQ ID NO:6 that does not overlap the first segment. Optionally the isolated nucleic acids are unique fragments of SEQ ID NO:1 or SEQ ID NO:6 or the complements of SEQ ID NO:1 or SEQ ID NO:6. The first and second isolated nucleic acid molecules are designed to act as primers capable of selectively amplifying at least a portion or all of SEQ ID NO:1 or SEQ ID NO:6. Preferably, the nucleic acid molecules that are used to detect and/or amplify the target sequence are selected from the group consisting of SEQ ID NO:7, SEQ ID NO:8, SEQ D NO:9, SEQ ID NO:10, SEQ ID NO:11, SEQ ID NO:12, SEQ ID NO:13, and SEQ ID NO:14.

Alternatively, protein based-kits are provided. Such kits include one or more binding polypeptides that selectively bind to a Salvador protein or to a Mutant Salvador protein, one or more control agents, and instructions for the use of the binding polypeptides, and agents in the diagnosis of a disorder associated with aberrant expression of a salvador molecule. In the preferred embodiments, the binding polypeptides are antibodies or antigen-binding fragments thereof, such as those described above. In these and other embodiments, certain of the binding polypeptides bind to the Mutant Salvador protein but do not bind to the Salvador protein to further distinguish the expression of these proteins in a biological sample.

The invention also provides treatment methods. In general, the treatment methods involve administering an agent to a subject to affect expression levels of a salvador molecule, e.g., to increase expression of a wild-type salvador molecule and/or reduce expression of a Mutant salvador molecule, and thereby treat the disorder. Thus, these methods include gene therapy applications. In certain embodiments, the method for treating a subject with a disorder characterized by aberrant expression of a salvador molecule, involves administering to the subject an effective amount of a salvador nucleic acid molecule to treat the disorder. In preferred embodiments, the salvador nucleic acid molecule has SEQ ID NO:1. In other embodiments, the method for treating a subject with a disorder characterized by aberrant expression of a salvador molecule involves administering to the subject an effective amount of a Salvador protein to treat the disorder. In yet other embodiments, the treatment method involves administering to the subject an effective amount of a binding agent (e.g., a nucleic acid or polypeptide) to inhibit a Mutant Salvador molecule and, thereby, treat the disorder. In certain preferred embodiments, the binding polypeptide is an antibody or an antigen-binding fragment thereof; more preferably, the antibodies or antigen-binding fragments are labeled with one or more cytotoxic agents. Successful treatment of the disorder is determined in accordance with standard clinical practice for assessing regression of the disorder being treated, e.g., a cancer, a birth defect, an autoimmunity disorder.

The invention provides various research methods and compositions. Thus, according to one aspect of the invention, a method for producing a Salvador protein is provided. The method involves providing a salvador nucleic acid molecule operably linked to a promoter, wherein the salvador nucleic acid molecule encodes the Salvador protein or a fragment thereof; expressing the salvador nucleic acid molecule in an expression system; and isolating the Salvador protein or a fragment thereof from the expression system. Preferably, the salvador nucleic acid molecule has SEQ ID NO:1 or SEQ ID NO:3. According to yet another aspect of the invention, a method for producing a Mutant Salvador protein is provided. This method involves: providing a Mutant salvador nucleic acid molecule operably linked to a promoter, wherein the Mutant salvador nucleic acid molecule encodes the Mutant Salvador protein or a fragment thereof; expressing the Mutant salvador nucleic acid molecule in an expression system; and isolating the Mutant Salvador protein or a fragment thereof from the expression system. Preferably, the Mutant salvador nucleic acid molecule has SEQ ID NO:1 or SEQ ID NO:3, with one or more point mutations, deletions, or truncations, to encode a Mutant Salvador protein.

According to other aspects of the invention, various research methods for using the compositions of the invention are provided. Exemplary research methods include a method to induce apoptosis in a cell by administering to the cell an effective amount of a salvador molecule to induce apoptosis in the cell. Exemplary research methods also include a method to inhibit cell proliferation in a cell by administering to the cell an effective amount of a salvador molecule to inhibit cell proliferation. Such methods can be performed in vivo or in vitro.

According to yet another aspect of the invention, a method for identifying a salvador molecule including a Mutant salvador molecule is provided. The method involves: (a) introducing a putative salvador molecule or a putative Mutant salvador molecule into a cell; and (b) detecting a salvador functional activity. The salvador functional activity is selected from the group consisting of binding to a warts/LATS molecule, modulating cell maturation/differentiation, modulating cell growth, modulating cell proliferation, and modulating cell death. In certain embodiments, the putative salvador molecule or putative Mutant salvador molecule is derived from a human cell, a Drosophila, or a nematode, e.g., C. elegans.

According to still another aspect of the invention, a method for identifying a salvador modulating agent that modulates a salvador molecule-cognate interaction is provided. The method involves (a) contacting a salvador molecule with a salvador molecule cognate, in the presence of a putative modulating agent, under conditions to allow the salvador molecule to bind to the cognate, e.g., a warts/LATS molecule, and (b) detecting salvador molecule binding to the cognate. A change in salvador molecule binding to the cognate in the presence of the putative modulating agent compared to salvador molecule binding to the cognate in the absence of the cognate indicates that the agent is a modulating agent of the salvador molecule and/or its cognate. The Salvador molulating agent may modulate the salvador molecule-cognate interaction by binding to one or both of these binding partners. In general, detecting comprises detecting a change in a parameter selected from salvador molecule binding to its cognate, cell maturation/differentiation, cell growth, cell proliferation, and/or cell death compared to the parameters detected in the absence of the putative modulating agent.

These and other aspects of the invention, as well as various advantages and utilities, will be more apparent with reference to the detailed description of the preferred embodiments.

### Abbreviated Sequence Listing

SEQ ID NO:1 is the human Salvador nucleic acid molecule (cDNA).
SEQ ID NO:2 is the human Salvador protein.
SEQ ID NO:3 is the Drosophila salvador melanogaster nucleic acid molecule (cDNA).
SEQ ID NO: 4 is the Drosophila Salvador protein.
SEQ ID NO: 5 is a probe for the human salvador gene.
SEQ ID NO: 6 is the human Salvador gene genomic DNA.
SEQ ID NO: 7 is a unique fragment of SEQ ID NO:1.
SEQ ID NO: 8 is a unique fragment of SEQ ID NO:1.
SEQ ID NO: 9 is a unique fragment of SEQ ID NO:1.
SEQ ID NO: 10 is a unique fragment of SEQ ID NO:1.
SEQ ID NO: 11 is a mutant Drosophila Salvador nucleic acid molecule, Salvador¹.
SEQ ID NO: 12 is a mutant Drosophila Salvador protein corresponding to Salvador¹.
SEQ ID NO: 13 is a mutant Drosophila salvador nucleic acid molecule, salvador².
SEQ ID NO: 14 is a mutant Drosophila Salvador protein corresponding to salvador².
SEQ ID NO: 15 is a mutant Drosophila Salvador nucleic acid molecule, salvador³.
SEQ ID NO: 16 is a mutant Drosophila Salvador protein corresponding to salvador³.
SEQ ID NO: 17 is LATS having GenBank accession number U29608.
SEQ ID NO: 18 is the LATS protein having GenBank accession number U29608.
SEQ ID NO: 19 is LATS having GenBank accession number L39837.
SEQ ID NO: 20 is the LATS protein having GenBank accession number L39837.
SEQ ID NO: 21 is LATS1 having GenBank accession number AF104413.
SEQ ID NO: 22 is the LATS1 protein having GenBank accession number AF104423.
SEQ ID NO: 23 is LATS2 having GenBank accession number AB028019.
SEQ ID NO: 24 is the LATS2 protein having GenBank accession number AB028019.
SEQ ID NO: 25 is a peptide used in a protein binding study.
SEQ ID NO: 26 is a peptide used in a protein binding study.

### BRIEF DESCRIPTION OF THE DRAWINGS

FIG. 1 illustrates the Salvador gene organization and protein sequence.

### DETAILED DESCRIPTION OF THE INVENTION

The invention is based, in part, on the discovery of a novel Drosophila gene, termed salvador, which is mutated in tumorous outgrowths. Three mutations were identified in the salvador locus that give mutant Drosophila cells a proliferative advantage with respect to wild type cells. The Drosophila salvador gene was cloned and found to encode a novel protein having two WW domains with highly conserved orthologues in C. elegans and mammals. The three salvador mutations identified in Drosophila result in proteins lacking both WW domains.

The two WW domains are located from nucleotide 1350 to 1448 and from nucleotide 1455 to 1553 of the Drosophila Salvador gene (SEQ ID NO: 3), corresponding to amino acids 424 to 456 and 459 to 491, respectively, of the Drosophila Salvador protein (SEQ ID NO: 4).

The human homologue of Salvador, hWW45 (GenBank accession number NM0218118), has been identified, but its function was previously unknown (Valverde, P., Biochem. Biophys. Res. Commun. 276: 990-998, 2000). In the hWW45 gene, the two WW domains are located from nucleotide 813 to 911 and from nucleotide 918 to 1016 of SEQ ID NO:1, corresponding to amino acids 200 to 232 and 235 to 267, resepectively, of the human Salvador protein (SEQ ID NO:2). The Drosophila and human Salvador gene products share 47% identity and 54% similarity over the C-termninal 188 amino acids.

The invention also is based, in part, on the discovery that the human counterpart of the Drosophila gene is deleted or mutated in at least three tumor-derived cancer cell lines. Accordingly, although not wishing to be bound to any particular theory or mechanism, we believe the salvador genes from various species play a role in modulating cellular development (including cell proliferation, growth, and death). Thus, the invention is directed to novel compositions of the human and Drosophila salvador nucleic acids and proteins encoded thereby (SEQ ID NOs: 1, 2, 3, and 4), as well as to agents that selectively bind to these novel molecules, and to diagnostic, therapeutic, and research applications of these compositions.

According to one aspect of the invention, an isolated nucleic acid molecule is provided. The isolated nucleic acid molecule is selected from the group consisting of:
(a) nucleic acid molecules which hybridize under stringent conditions to a nucleic acid molecule having a nucleotide sequence set forth as SEQ ID NO:1 or SEQ ID NO:3, and which code for a Salvador protein,
(b) deletions, additions and substitutions of the nucleic acid molecules of (a), which code for a Salvador protein,
(c) nucleic acid molecules that differ from the nucleic acid molecules of (a) or (b) in codon sequence due to the degeneracy of the genetic code, and
(d) complements of (a), (b), or (c).

The preferred isolated nucleic acids of the invention are salvador nucleic acid molecules which encode a Salvador protein. As used herein, a Salvador protein refers to a protein which is encoded by a nucleic acid having SEQ ID NO:1 or SEQ ID NO:3, or a functional fragment thereof, or a functional equivalent thereof (e.g., a nucleic acid sequence encoding the same protein as encoded by SEQ ID NO:1 or SEQ ID NO:3), provided that the functional fragment or equivalent encodes a protein which exhibits a salvador functional activity. As used herein, a salvador functional activity refers to the ability of a salvador molecule to modulate cellular development such as cell maturation/differentiation, cell proliferation, growth, and death. An exemplary salvador functional activity is a tumor suppressor activity which includes suppressing and/or reducing tumor cell growth, proliferation, and/or metastasis. Although not wishing to be bound to any particular theory or mechanism, it is believed that the salvador molecule affects cellular development by promoting cellular arrest in the G₁ phase of the cell cycle, promoting cell cycle exit, and/or regulating cell death. An assay to measure salvador functional activity is described in the Examples. Specifically, a salvador molecule is said to have functional activity if the gene product or protein is capable of detectably binding to a protein with a PPPY motif.

In the preferred embodiments, the isolated nucleic acid molecule is SEQ ID NO:1 or SEQ ID NO:3. The invention provides nucleic acid molecules which code for Salvador proteins and which hybridize under stringent conditions to a nucleic acid molecule consisting of the nucleotide set forth in SEQ ID NO:1 or SEQ ID NO:3. Such nucleic acids may be DNA, RNA, or composed of mixed deoxyribonucleotides and ribonucleotides, and may also incorporate synthetic non-natural nucleotides. Various methods for determining the expression of a nucleic acid and/or a polypeptide in normal and tumor cells are known to those of skill in the art and are described further below and in the Examples. As used herein, the term “protein” is meant to include high molecular weight proteins, polypeptides, low molecular weight peptides, and fragments thereof.

The term “stringent conditions,” as used herein, refers to parameters with which one skilled in the art is familiar. Nucleic acid hybridization parameters may be found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄ (pH 7), 0.5% SDS, 2 mM EDTA). SSC is 0.15M sodium chloride/0.15M sodium citrate, pH 7; SDS is sodium dodecyl sulphate; and EDTA is ethylenediaminetetracetic acid. After hybridization, the membrane upon which the DNA is transferred is washed at 2×SSC at room temperature and then at 0.1×SSC/0.1% SDS at temperatures up to 68° C. The foregoing set of hybridization conditions is but one example of stringent hybridization conditions known to one of ordinary skill in the art. There are other conditions, reagents, and so forth which can be used, which also result in stringent hybridization. The skilled artisan will be familiar with such conditions, and thus they are not given here. It will be understood, however, that the skilled artisan will be able to manipulate the conditions in a manner to permit the clear identification of homologs and alleles of salvador nucleic acid molecules of the invention. The skilled artisan also is familiar with the methodology for screening cells and libraries for expression of such molecules which then are routinely isolated, followed by isolation of the pertinent nucleic acid molecule and sequencing.

In general, homologs and alleles typically will share at least 35% nucleotide identity and/or at least 40% amino acid identity and/or 50% similarity to SEQ ID NOs:1 or 3 and SEQ ID NOs:2 or 4, respectively, in some instances will share at least 45% nucleotide identity and/or at least 50% amino acid identity and/or 60% similarity, and in still other instances will share at least 55% nucleotide identity and/or at least 60%-75% amino acid identity and/or 70%-85% similarity. Preferred homologs and alleles share nucleotide and amino acid identities with SEQ ID NO:1 or SEQ ID NO:3 and SEQ ID NO:2 or SEQ ID NO:4, respectively, and encode polypeptides of greater than 70%, more preferably greater than 80%, still more preferably greater than 90% and most preferably greater than 99% identity. The percent identity can be calculated using various, publicly available software tools developed by NCBI (Bethesda, Md.) that can be obtained through the internet (ftp:/ncbi.nlm.nih.gov/pub/). Exemplary tools include the BLAST system, available at http://www.ncbi.nlm.nih.gov, which uses algorithms developed by Altschul et al. (Nucleic Acids Res. 25:3389-3402, 1997). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acid molecules also are embraced by the invention.

In screening for salvador nucleic acid molecules, a Southern blot may be performed using the foregoing conditions, together with a radioactive probe. After washing the membrane to which the DNA is finally transferred, the membrane can be placed against X-ray film to detect the radioactive signal.

The invention also includes degenerate nucleic acid molecules which include alternative codons, or triplets, to those present in the native materials. For example, serine is encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into a Salvador protein. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

According to another aspect of the invention, further isolated nucleic acid molecules that are based on the above-noted salvador nucleic acid molecules are provided. In this aspect, the isolated nucleic acid molecules are selected from the group consisting of:
(a) a unique fragment of the nucleotide sequence set forth as SEQ ID NO:1 or set forth as SEQ ID NO:3 between 12 and 2000 (more preferably, between 12 and 1400 nucleotides in length and, most preferably, between 12 and 32 nucleotides in length) or more and
(b) complements of (a),
wherein the unique fragments exclude nucleic acids having nucleotide sequences that are contained within SEQ ID NO:1 or SEQ ID NO:3, and that are known as of the priority date of this application (Table 1).

Known fragments, i.e., fragments that are not unique, of SEQ ID NO:1 and SEQ ID NO:3 are provided in Table 1 and identified by their positions in SEQ ID NO:1 and SEQ ID NO:3. Overlapping fragments are identified by a range of starting and ending nucleotides (Table 1). For example, a fragment which begins with any nucleotide between 1905 and 1916 of SEQ. ID NO:1 and ends with any nucleotide between 1930 and 1955 of SEQ ID NO:1 is not a unique fragment.

TABLE 1
Nucleotide sequence contained within SEQ ID NO: 1 and SEQ ID
NO: 3 that are known as of the priority date of this application

| Starting nucleotide(s) | Ending nucleotide(s) | Sequence |
|---|---|---|
| 1 | 68 | SEQ ID NO: 1 |
| 38 | 68 | SEQ ID NO: 1 |
| 158 | 309-311 | SEQ ID NO: 1 |
| 174 | 1707 | SEQ ID NO: 1 |
| 310 | 406 | SEQ ID NO: 1 |
| 749 | 1021 | SEQ ID NO: 1 |
| 1019 | 1165 | SEQ ID NO: 1 |
| 1720 | 1738 | SEQ ID NO: 1 |
| 1748 | 2009 | SEQ ID NO: 1 |
| 1755 | 2009 | SEQ ID NO: 1 |
| 1905-1916 | 1930-1955 | SEQ ID NO: 1 |
| 1921-1937 | 1947-1961 | SEQ ID NO: 1 |
| 1974 | 1999 | SEQ ID NO: 1 |
| 1980-1985 | 1998-2010 | SEQ ID NO: 1 |
| 1994 | 2005 | SEQ ID NO: 1 |
| 2029 | 2148-2151 | SEQ ID NO: 1 |
| 2105 | 2118 | SEQ ID NO: 1 |
| 2107 | 2120 | SEQ ID NO: 1 |
| 2047-2063 | 2069-2083 | SEQ ID NO: 1 |
| 1 | 767 | SEQ ID NO: 3 |
| 1 | 892 | SEQ ID NO: 3 |
| 144 | 163 | SEQ ID NO: 3 |
| 155 | 172 | SEQ ID NO: 3 |
| 158 | 175 | SEQ ID NO: 3 |
| 172 | 190 | SEQ ID NO: 3 |
| 372 | 391 | SEQ ID NO: 3 |
| 386 | 403 | SEQ ID NO: 3 |
| 410-413 | 431-433 | SEQ ID NO: 3 |
| 584 | 603 | SEQ ID NO: 3 |
| 765-771 | 784-791 | SEQ ID NO: 3 |
| 773 | 892 | SEQ ID NO: 3 |
| 958 | 1846 | SEQ ID NO: 3 |
| 1192 | 1210 | SEQ ID NO: 3 |
| 1378 | 1397 | SEQ ID NO: 3 |
| 1424 | 1442 | SEQ ID NO: 3 |
| 1467 | 1484 | SEQ ID NO: 3 |
| 1528 | 1547 | SEQ ID NO: 3 |
| 1812 | 1829-1830 | SEQ ID NO: 3 |
| 1846 | 2194 | SEQ ID NO: 3 |
| 1881-1883 | 1903-1904 | SEQ ID NO: 3 |

Thus, the invention provides isolated unique fragments of SEQ ID NOs:1 or 3 or complements of SEQ ID NOs:1 or 3. A unique fragment is one that is a ‘signature’ for the larger nucleic acid. It, for example, is long enough to assure that its precise sequence is not found in molecules outside of the Salvador nucleic acid molecules defined above. Those of ordinary skill in the art may apply no more than routine procedures to determine if a fragment is unique within the human or Drosophila genome. Unique fragments, however, exclude fragments completely composed of the nucleotide sequences that are contained within SEQ ID NO: 1 or SEQ ID NO: 3 and that are known as of the priority date of this application.

Unique fragments can be used as probes in Southern blot assays to identify such nucleic acid molecules, or can be used as probes in amplification assays such as those employing the polymerase chain reaction (PCR). As known to those skilled in the art, large probes such as 200 nucleotides or more are preferred for certain uses such as Southern blots, while smaller fragments will be preferred for uses such as PCR. Especially preferred unique fragments include the sequences set forth in SEQ ID NO:7, SEQ ID NO:8, SEQ ID NO:9, SEQ ID NO:10, SEQ ID NO:11, SEQ ID NO:12, SEQ ID NO:13, and SEQ ID NO:14. Unique fragments also can be used to produce fusion proteins for generating antibodies or determining binding of the polypeptide fragments, or for generating immunoassay components. Likewise, unique fragments can be employed to produce nonfused fragments of the Salvador polypeptides useful, for example, in the preparation of antibodies, in immunoassays.

As will be recognized by those skilled in the art, the size of the unique fragment will depend upon its conservancy in the genetic code. Thus, some regions of SEQ ID NO:1 and/or SEQ ID NO:3 and its complement will require longer segments to be unique while others will require only short segments, typically between 12 and 32 nucleotides or more in length (e.g. 12, 13, 14, 15, 16, 17, 18, 19, 20, 21, 22, 23, 24, 25, 26, 27, 28, 29, 30, 31, and 32 or more), up to the entire length of the disclosed sequence. Many segments of the polynucleotide coding region or complements thereof that are 18 or more nucleotides in length will be unique. Those skilled in the art are well versed in methods for selecting such sequences, typically on the basis of the ability of the unique fragment to selectively distinguish the sequence of interest from non-salvador nucleic acid molecules. A comparison of the sequence of the fragment to those on known data bases typically is all that is necessary, although in vitro confirmatory hybridization and sequencing analysis may be performed.

A unique fragment can be a functional fragment. A functional fragment of a nucleic acid molecule of the invention is a fragment which retains some functional property of the larger nucleic acid molecule, such as coding for a functional polypeptide, binding to proteins, regulating transcription of operably linked nucleic acid molecules, and the like. One of ordinary skill in the art can readily determine using the assays described herein and those well known in the art to determine whether a fragment is a functional fragment of a nucleic acid molecule using no more than routine experimentation. Preferred functional fragments include fragments comprising one or more WW domains of the salvador gene.

According to yet another aspect of the invention, isolated nucleic acid molecules that are based on the salvador gene genomic region (SEQ ID NO:6) are provided. According to this aspect of the invention, the isolated nucleic acid molecule is selected from the group consisting of:
(a) nucleic acid molecules which hybridize under stringent conditions to a nucleic acid molecule having a nucleotide sequence set forth as SEQ ID NO:6 (human salvador genomic DNA), and which codes for a Salvador protein,
(b) deletions, additions and substitutions of the nucleic acid molecules of (a), which code for a Salvador protein,
(c) nucleic acid molecules that differ from the nucleic acid molecules of (a) or (b) in codon sequence due to the degeneracy of the genetic code, and
(d) complements of (a), (b) or (c).
   The preferred isolated nucleic acids of the invention are salvador nucleic acid molecules which encode a Salvador protein as defined above.

According to yet another aspect of the invention, unique fragments of the salvador gene genomic sequence are provided. In this aspect, the isolated nucleic acid molecules are selected from the group consisting of:
(a) a unique fragment of the nucleotide sequence set forth as SEQ ID NO:6 between 12 and 2000 nucleotides in length or more and
(b) complements of (a),
   wherein the unique fragments exclude nucleic acids having nucleotide sequences that are contained within. SEQ ID NO:6, and that are known as of the priority date of this application. In preferred embodiments, the nucleic acid molecule is selected from the group consisting of: an exon of SEQ ID NO:6, a unique fragment of an exon of SEQ ID NO:6, an intron of SEQ ID NO:6, a unique fragment of an intron of SEQ ID NO:6, and complements of the foregoing. Exons of SEQ ID NO:6 include nucleotides 3171-3479 (exon 1), nucleotides 5733-6173 (exon 2), nucleotides 25027-25297 (exon 3), nucleotides 27622-27765 (exon 4), and nucleotides 33131-34873 (exon 5). In a particularly preferred embodiment, the isolated nucleic acid molecule comprises SEQ ID NO:5. Preferably, the unique fragments are between 12 and 2000 nucleotides in length, more preferably between 12 and 1400 nucleotides in length.

In yet another aspect of the invention, Mutant salvador nucleic acid molecules are provided. The Mutant salvador nucleic acid molecules contain a sequence which is identical to SEQ ID NO:1 or SEQ ID NO:3, with the exception that the sequence includes one or more mutations, e.g., deletions, additions, substitutions, or truncations, such that the Mutant salvador nucleic acid molecule does not encode a functional Salvador protein. Rather, the Mutant salvador nucleic acid molecules encode a Mutant Salvador protein, i.e., a protein which does not exhibit a Salvador protein functional activity. Typically, there are fewer than 30 deletions, additions, substitutions, truncations, or combinations thereof. In some embodiments, there are 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 mutation(s).

Three Mutant salvador nucleic acid molecules were identified in Drosophila using a genetic screen, as described in the Examples. The Mutant salvador nucleic acid sequences salvador¹, salvador², and salvador³ are set forth in SEQ ID NO: 11, SEQ ID NO: 13, and SEQ ID NO: 15, respectively. The predicted sequences of Mutant proteins corresponding to the foregoing genes are set forth in SEQ ID NO: 12, SEQ ID NO: 14, and SEQ ID NO: 16 respectively. These four Mutant salvador genes include mutations which prohibit at least nucleotides 1350-1448 and 1455-1553 of SEQ ID NO:3 from being expressed, resulting in Mutant Salvador protein sequences that are truncated or otherwise mutated proteins lacking both WW domains characteristic of WW domain proteins. Preferred human Mutant salvador nucleic acid molecules have a nucleic acid sequence that is a truncated form of SEQ ID NO:1. These Mutant salvador nucleic acid molecules result in Mutant human Salvador proteins lacking one or more WW domains, which are located between amino acids 200 to 232 and 235 to 267 of SEQ ID NO:2.

As used herein, with respect to nucleic acid molecules, the term “isolated” means: (i) amplified in vitro by, for example, PCR; (ii) recombinantly produced by cloning; (iii) purified, for example, by cleavage and gel separation; or (iv) synthesized, for example, chemically. An isolated nucleic acid molecule is one which is readily manipulable by recombinant DNA techniques well known in the art. Thus, a nucleotide sequence contained in a vector in which 5′ and 3′ restriction sites are known or for which PCR primer sequences have been disclosed is considered isolated but a nucleic-acid sequence existing in its native state in its natural host is not. An isolated nucleic acid molecule may be substantially purified, but need not be. For example, a nucleic acid molecule that is isolated within a cloning or expression vector is not pure in that it may comprise only a tiny percentage of the material in the cell in which it resides. Such a nucleic acid molecule is isolated, however, as the term is used herein, because it is readily manipulable by standard techniques known to those of ordinary skill in the art. An isolated nucleic acid molecule, as used herein, does not include a naturally occurring chromosome.

As used herein, a “Mutant Salvador nucleic acid molecule” refers to a Salvador nucleic acid molecule which includes a mutation (point mutation, addition, deletion, rearrangement, substitution, truncation, and the like) such that the Mutant salvador nucleic acid molecule does not encode a functional Salvador protein. Rather, the Mutant salvador nucleic acid molecule encodes a Mutant Salvador protein, i.e., a protein which does not exhibit a Salvador protein functional activity. A “Mutant Salvador protein” refers to a Salvador protein that is a protein product of a Mutant salvador nucleic acid molecule which includes a mutation that affects the functional activity of the salvador molecule. Preferred Mutant Salvador proteins are those which lack one or more WW domains and/or one or more functional WW domains, e.g., the protein can be a truncated form of the Salvador protein having SEQ ID NO:2.

As used herein, the term “aberrant expression” refers to either or both of decreased expression (including no detectable expression) of a salvador molecule (nucleic acid or protein) or increased expression of a Mutant salvador molecule (nucleic acid or protein). The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

According to yet another aspect of the invention, an expression vector comprising any of the isolated nucleic acid molecules of the invention, preferably operably linked to a promoter is provided. In a related aspect, host cells transformed or transfected with such expression vectors also are provided.

As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids, and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, e.g., β-galactosidase or alkaline phosphatase, and genes which visibly affect the phenotype of transformed or transfected cells, hosts, colonies or plaques, e.g., green fluorescent protein. Preferred vectors are those capable of autonomous replication and expression of the structural gene products present in the DNA segments to which they are operably joined.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

It will also be recognized that the invention embraces the use of the salvador cDNA and genomic sequences or Mutant Salvador cDNA and genomic sequences in expression vectors, as well as to transfect host cells and cell lines, be these prokaryotic, e.g., E. coli or eukaryotic, e.g., CHO cells, COS cells, yeast expression systems, and recombinant baculovirus expression in insect cells. Especially useful are mammalian cells such as human, mouse, hamster, pig, goat, primate, etc. They may be of a wide variety of tissue types, including mast cells, fibroblasts, oocytes, and lymphocytes, and may be primary cells and cell lines. Specific examples include dendritic cells, U293 cells keratinocytes, peripheral blood leukocytes, bone marrow stem cells and embryonic stem cells. The expression vectors require that the pertinent sequence, i.e., those nucleic acids described supra, be operably linked to a promoter.

According to still a further aspect of the invention, a transgenic non-human animal comprising an expression vector of the invention is provided, including a transgenic non-human animal which has reduced or increased expression of a salvador nucleic acid molecule or elevated expression of a Mutant salvador nucleic acid molecule.

As used herein, the term “transgenic non-human animals” includes non-human animals having one or more exogenous nucleic acid molecules incorporated in germ line cells and/or somatic cells. Thus, the transgenic animals include “knockout” animals having a homozygous or heterozygous gene disruption by homologous recombination, animals having episomal or chromosomally incorporated expression vectors, etc. Knockout animals can be prepared by homologous recombination using embryonic stem cells as is well known in the art. The recombination can be facilitated by the cre/lox system or other recombinase systems known to one of ordinary skill in the art. In certain embodiments, the recombinase system itself is expressed conditionally, for example, in certain tissues or cell types, at certain embryonic or post-embryonic developmental stages, inducibly by the addition of a compound which increases or decreases expression, and the like. In general, the conditional expression vectors used in such systems use a variety of promoters which confer the desired gene expression pattern, e.g., temporal or spatial patterns. Conditional promoters also can be operably linked to salvador nucleic acid molecules to increase or decrease expression of a salvador molecule in a regulated or conditional manner. Trans-acting negative or positive regulators of salvador activity or expression also can be operably linked to a conditional promoter as described above. Such trans-acting regulators include antisense salvador nucleic acid molecules, nucleic acid molecules which encode dominant negative salvador molecules, ribozyme molecules specific for salvador nucleic acid molecules, and the like. The transgenic non-human animals are useful in experiments directed toward testing biochemical or physiological effects of diagnostics or therapeutics for conditions characterized by increased or decreased salvador molecule expression. Other uses will be apparent to one of ordinary skill in the art. Thus, the invention also permits the construction of salvador gene “knockouts” in cells and in animals, providing materials for studying certain aspects of cellular development including, e.g., modulating cell maturation/differentiation, cell growth, cell proliferation, and cell death, as well as metastasis.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA or RNA encoding a Salvador protein, a Mutant Salvador protein, fragments, or variants thereof. The heterologous DNA or RNA is placed under operable control of transcriptional elements to permit the expression of the heterologous DNA in the host cell.

Preferred systems for mRNA expression in mammalian cells are those such as pRc/CMV (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mizushima and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant is described by Warnier et al., in intradermal injection in mice for immunization against P1A (Int. J. Cancer, 67:303-310, 1996).

The invention also embraces kits termed expression kits, which allow the artisan to prepare a desired expression vector or vectors. Such expression kits include at least separate portions of each of the previously discussed coding sequences. Other components may be added, as desired, as long as the previously mentioned sequences, which are required, are included.

According to another aspect of the invention, an isolated protein encoded by any of the foregoing isolated nucleic acid molecules of the invention is provided. Preferably, the isolated protein comprises SEQ ID NO: 2 or SEQ ID NO: 4, for example, fragments comprising at least one WW domain. The invention also embraces Mutant Salvador proteins, such as those described in the Examples and other Mutant Salvador proteins such as truncated forms of SEQ ID NO:2 which lack one or more functional WW domains.

The invention also provides isolated proteins, which include the proteins of SEQ ID NOs:2 and 4 and unique fragments of SEQ ID NOs:2 and 4, for example, fragments comprising at least one functional WW domain. Such proteins are useful, for example, alone or as fusion proteins to generate antibodies (for example, as therapeutics), or as a component(s) of an immunoassay.

As used herein, a Salvador protein refers to a protein which is encoded by a nucleic acid having SEQ ID NO:1 or SEQ ID NO:3, a functional fragment thereof, or a functional equivalent thereof (e.g., a nucleic acid sequence encoding the same protein as encoded by SEQ ID NO:1 or SEQ ID NO:3), provided that the functional fragment or equivalent encodes a Salvador protein which exhibits a salvador functional activity. As used herein, a “salvador functional activity” refers to the ability of a Salvador protein to modulate cellular development, e.g., cell maturation/differentiation, cell growth, cell proliferation, and cell death. Modulating cellular development can be detected using conventional assays which detect any one or more of cell maturation/differentiation, cell growth, proliferation (including, e.g., cell metastasis), and death (including, e.g., apoptosis assays). Thus, an exemplary salvador functional activity is a tumor suppressor activity such as suppressing and/or reducing tumor cell growth, proliferation, and/or metastasis. Although not wishing to be bound to any particular theory or mechanism, it is believed that the Salvador protein modulates at least some of the above-noted cell functions by promoting cellular arrest in the G₁ phase of the cell cycle, promoting cell cycle exit, and/or regulating cell death.

Proteins can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, including antigenic peptides (such as are presented by MHC molecules on cell surfaces for immune recognition) also can be synthesized chemically using well-established methods of peptide synthesis.

Thus, as used herein with respect to proteins, “isolated” means separated from its native environment and present in sufficient quantity to permit its identification or use. Isolated, when referring to a protein or polypeptide, means, for example: (i) selectively produced by expression of a recombinant nucleic acid or (ii) purified as by chromatography or electrophoresis. Isolated proteins or polypeptides may, but need not be, substantially pure. The term “substantially pure” means that the proteins or polypeptides are essentially free of other substances with which they may be found in nature or in vivo systems to an extent practical and appropriate for their intended use. Substantially pure proteins may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, e.g., isolated from other proteins.

A fragment of a Salvador protein, for example, generally has the features and characteristics of fragments including unique fragments as discussed above in connection with nucleic acid molecules. As will be recognized by those skilled in the art, the size of a fragment which is unique will depend upon factors such as whether the fragment constitutes a portion of a conserved protein domain. Thus, some regions of Salvador proteins will require longer segments to be unique while others will require only short segments, typically between 5 and 12 amino acids (e.g., 5, 6, 7, 8, 9, 10, 11, and 12 amino acids long).

Unique fragments of a protein preferably are those fragments which retain a distinct functional capability of the protein. Functional capabilities which can be retained in a fragment of a protein include interaction with antibodies, interaction with other proteins or fragments thereof, selective binding of nucleic acid molecules, and enzymatic activity. One important activity is the ability to act as a signature for identifying the polypeptide. Another is the ability to provoke in an animal an immune response to a Mutant salvador molecule but not provoke an immune response to a salvador molecule and, thereby, create antibodies that are selective for the Mutant salvador molecule.

Those skilled in the art are well versed in methods for selecting fragments with unique amino acid sequences, typically on the basis of the ability of the fragment to selectively distinguish the sequence of interest from non-family members. A comparison of the sequence of the fragment to those in known data bases typically is all that is necessary.

The invention embraces variants and mutants of the Salvador proteins described herein. As used herein, a “variant” of a Salvador protein is a protein which contains one or more modifications to the primary amino acid sequence of a Salvador protein. Modifications which create a Salvador protein variant can be made to a Salvador protein (1) to produce, increase, reduce, or eliminate an activity of the Salvador protein; (2) to enhance a property of the Salvador protein, such as protein stability in an expression system or the stability of protein-protein binding; or (3) to provide a novel activity or property to a Salvador protein, such as addition of an antigenic epitope or addition of a detectable moiety. Modifications to a Salvador protein or to a Mutant Salvador protein are typically made to the nucleic acid molecule which encodes the protein, and can include deletions, point mutations, truncations, rearrangements, amino acid substitutions, and additions of amino acids or non-amino acid moieties.

Mutations of a nucleic acid molecule which encode a Salvador protein preferably preserve the amino acid reading frame of the coding sequence, and preferably do not create regions in the nucleic acid which are likely to hybridize to form secondary structures, such as hairpins or loops, which can be deleterious to expression of the variant protein.

Mutations can be made by selecting an amino acid substitution, or by random mutagenesis of a selected site in a nucleic acid which encodes the protein. Variant proteins are then expressed and tested for one or more activities to determine which mutation provides a variant protein with the desired properties. Further mutations can be made to variants (or to non-variant Salvador proteins) which are silent as to the amino acid sequence of the protein, but which provide preferred codons for translation in a particular host. The preferred codons for translation of a nucleic acid in, e.g., E. coli are well known to those of ordinary skill in the art. Still other mutations can be made to the noncoding sequences of a salvador gene or cDNA clone to enhance expression of the protein. The activity of variants of Salvador proteins can be tested by cloning the gene encoding the variant Salvador protein into a bacterial or mammalian expression vector, introducing the vector into an appropriate host cell, expressing the variant Salvador protein, and testing for a functional capability of the Salvador protein as disclosed herein.

Alternatively, modifications can be made directly to the Salvador protein, such as by cleavage, addition of a linker molecule, addition of a detectable moiety, such as biotin, addition of a fatty acid, and the like. Modifications also embrace fusion proteins comprising all or part of the salvador amino acid sequences, or proteins which retain the same conformation and/or function but have a designed sequence. One of skill in the art will be familiar with methods for designing a protein sequence for a particular protein conformation, and can thus “design” a variant Salvador polypeptide according to known methods. One example of such a method is described by Dahiyat and Mayo in Science 278:82-87, 1997, and U.S. Patent Nos. 6,188,965 and 6,269,312, whereby proteins or portions thereof can be designed de novo. These methods can also be applied to a known protein to vary only a portion of the protein sequence. By applying computational methods, specific variants of a Salvador protein can be proposed and tested to determine whether the variant retains a desired conformation and/or function. In general, variants include Salvador proteins which are modified specifically to alter a feature of the protein unrelated to its desired physiological activity. For example, cysteine residues can be substituted or deleted to prevent unwanted disulfide linkages. Similarly, certain amino acids can be changed to enhance expression of a Salvador protein by eliminating proteolysis by proteases in an expression system, e.g., dibasic amino acid residues in yeast expression systems in which KEX2 protease activity is present. Additionally, some or all of the protein may be redesigned to, for example, enhance stability or shelf life.

The skilled artisan will also realize that conservative amino acid substitutions may be made in Salvador proteins to provide functional variants of the foregoing proteins, i.e., the variants retain the functional capabilities of the Salvador proteins. As used herein, a “conservative amino acid substitution” refers to an amino acid substitution which does not alter the relative charge or size characteristics of the protein in which the amino acid substitution is made. Conservative substitutions of amino acids include substitutions made amongst amino acids within the following groups: (a) M, 1, L, V; (b) F, Y, W; (c) K, R, H; (d) A, G; (e) S, T; (f) Q, N; and (g) E, D.

For example, upon determining that a peptide derived from a Salvador protein plays a role in cellular development, e.g., tumor suppression, metastasis, cell maturation/differentiation, cell growth, cell proliferation, cell death, and/or apoptosis, one can make conservative amino acid substitutions to the amino acid sequence of the peptide. The substituted peptides can then be tested for one or more of the above-noted functions, in vivo or in vitro. These variants can be tested for improved stability and are useful, inter alia, in pharmaceutical compositions.

Functional variants of Salvador proteins, i.e., variants of proteins which retain the function of the Salvador proteins, can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functional variants of the Salvador proteins include conservative amino acid substitutions of proteins encoded by SEQ ID NOs:2 or 4. Conservative amino acid substitutions in the amino acid sequence of Salvador proteins to produce functional variants of Salvador proteins typically are made by alteration of the nucleic acid molecule encoding a Salvador protein (e.g., SEQ ID NO:1 or SEQ ID NO:3). Such substitutions can be made by a variety of methods known to one of ordinary skill in the art. For example, amino acid substitutions may be made by PCR-directed mutations, site-directed mutagenesis according to the method of Kunkel (Kunkel, Proc. Nat. Acad. Sci. U.S.A. 82: 488-492, 1985), or by chemical synthesis of a gene encoding a Salvador protein. Where amino acid substitutions are made to a small unique fragment of a Salvador protein, the substitutions can be made by directly synthesizing the peptide. The activity of functional variants or fragments of Salvador protein can be tested by cloning the gene encoding the altered Salvador protein into a bacterial or mammalian expression vector, introducing the vector into an appropriate host cell, expressing the altered Salvador protein, and testing for a functional capability of the Salvador protein as disclosed herein.

The invention as described herein has a number of uses, some of which are described elsewhere herein. First, the invention permits isolation of the Salvador proteins and Mutant Salvador proteins. A variety of methodologies well-known to the skilled practitioner can be utilized to obtain isolated Salvador proteins and Mutant Salvador proteins. The proteins may be purified from cells which naturally produce the protein by chromatographic means or immunological recognition. Alternatively, an expression vector may be introduced into cells to cause production of the protein. In another method, mRNA transcripts may be microinjected or otherwise introduced into cells to cause production of the encoded protein. Translation of mRNA in cell-free extracts such as the reticulocyte lysate system also may be used to produce the protein. Those skilled in the art also can readily follow known methods for isolating Salvador proteins and Mutant Salvador proteins. These include, but are not limited to, chromatographic techniques such as immunochromatography, HPLC, size-exclusion chromatography, ion-exchange chromatography, and immune-affinity chromatography.

The isolation and identification of salvador nucleic acid molecules and of Mutant salvador nucleic acid molecules also allows one of skill in the art to diagnose a disorder characterized by aberrant expression of a salvador nucleic acid molecule or protein or of a Mutant salvador nucleic acid molecule or protein. These methods involve determining the aberrant expression of one or more Salvador nucleic acid molecules and/or Mutant Salvador nucleic acid molecules, and/or encoded Salvador proteins and/or Mutant Salvador proteins. In the former two situations, such determinations can be carried out via any standard nucleic acid determination assay, including the polymerase chain reaction, or assaying with hybridization probes which may be labeled. In the latter two situations, such determinations can be carried out by assaying biological samples with binding partners (e.g., antibodies) for Salvador proteins or Mutant Salvador proteins.

The invention also provides, in certain embodiments, “dominant negative” polypeptides derived from Salvador proteins and/or Mutant Salvador proteins. A dominant negative polypeptide is an inactive variant of a protein, which, by interacting with the cellular machinery, displaces an active protein from its interaction with the cellular machinery or competes with the active protein, thereby reducing the effect of the active protein. Dominant negative polypeptides are useful, or example, for preparing transgenic non-human animals to further characterize the functions of the salvador molecules and Mutant salvador molecules disclosed herein. For example, a dominant negative receptor which binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to a promoter site in the control region of a gene but does not increase gene transcription can reduce the effect of a normal transcription factor by occupying promoter binding sites without increasing transcription.

The end result of the expression of a dominant negative polypeptide in a cell is a reduction in function of active proteins. One of ordinary skill in the art can assess the potential for a dominant negative variant of a protein, and using standard mutagenesis techniques to create one or more dominant negative variant polypeptides. For example, one of ordinary skill in the art can modify the sequence of Salvador proteins by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. The skilled artisan then can test the population of mutagenized proteins for diminution in a selected and/or for retention of such an activity. Other similar methods for creating and testing dominant negative variants of a protein will be apparent to one of ordinary skill in the art.

In yet a further aspect of the invention, binding polypeptides that selectively bind to a salvador molecule and/or to a Mutant salvador molecule are provided. According to this aspect, the binding polypeptides bind to an isolated nucleic acid or protein of the invention, including unique fragments thereof. Preferably, the binding polypeptides bind to a Salvador protein, a Mutant Salvador protein, or a unique fragment thereof. In certain particularly preferred embodiments, the binding polypeptide binds to a Mutant Salvador protein but does not bind to a Salvador protein, i.e., the binding polypeptides are selective for binding to the Mutant Salvador protein and can be used in various assays to detect the presence of the Mutant Salvador protein without detecting Salvador protein. Such Mutant Salvador protein binding polypeptides also can be used to selectively bind to a Mutant salvador molecule in a cell (in vivo or ex vivo) for imaging and therapeutic applications in which, for example, the binding polypeptide is tagged with a detectable label and/or a toxin for targeted delivery to the salvador molecule. Of course, other binding polypeptides can be developed which bind selectively to the Salvador protein and not to the Mutant Salvador protein to, for example, selectively identify expression of a Salvador protein in a cell.

In preferred embodiments, the binding polypeptide is an antibody or antibody fragment, more preferably, an Fab or F(ab)₂ fragment of an antibody. Typically, the fragment includes a CDR3 region that is selective for the Salvador protein and/or Mutant Salvador protein. Any of the various types of antibodies can be used for this purpose, including polyclonal antibodies, monoclonal antibodies, humanized antibodies, and chimeric antibodies.

Thus, the invention provides agents which bind to Salvador proteins and/or Mutant Salvador proteins encoded by salvador nucleic acid molecules and/or Mutant salvador nucleic acid molecules, respectively, and in certain embodiments preferably to unique fragments of the Salvador proteins or Mutant Salvador proteins. Such binding partners can be used in screening assays to detect the presence or absence of a Salvador protein and/or a Mutant Salvador protein and in purification protocols to isolate such Salvador proteins and/or Mutant Salvador proteins. Likewise, such binding partners can be used to selectively target drugs, toxins or other molecules to cells which express Salvador and/or Mutant Salvador proteins. In this manner, for example, cells present in solid or non-solid tumors which express Mutant Salvador proteins can be treated with cytotoxic compounds that are selective for the Mutant Salvador molecules (nucleic acids and/or proteins). Such binding agents also can be used to inhibit the native activity of the Salvador and/or Mutant Salvador proteins, for example, by binding to such proteins, to further characterize the functions of these molecules.

The invention, therefore, provides antibodies or fragments of antibodies having the ability to selectively bind to Salvador proteins and/or Mutant Salvador proteins, and preferably to unique fragments of the foregoing. Antibodies include polyclonal, monoclonal, and chimeric antibodies, prepared, e.g., according to conventional methodology.

The antibodies of the present invention thus are prepared by any of a variety of methods, including administering a protein, fragments of a protein, cells expressing the protein or fragments thereof and the like to an animal to induce polygonal antibodies. The production of monoclonal antibodies is according to techniques well known in the art. As detailed herein, such antibodies may be used for example to identify tissues expressing protein or to purify protein. Antibodies also may be coupled to specific labeling agents for imaging or to antitumor agents, including, but not limited to, methotrexate, radioiodinated compounds, toxins such as ricin, other cytostatic or cytolytic drugs, and so forth.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the present invention also provides for F(ab′)₂, Fab, Fv, and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies. Thus, the invention involves polypeptides of numerous size and type that bind specifically to Salvador proteins and/or Mutant Salvador proteins. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties.

Phage display can be particularly effective in identifying binding peptides useful according to the invention. Briefly, one prepares a phage library (using, e.g., m13, fd, or lambda phage), displaying inserts from 4 to about 80 amino acid residues using conventional procedures. The inserts may represent a completely degenerate or biased array. One then can select phage-bearing inserts which bind to a Salvador protein or a Mutant Salvador protein. This process can be repeated through several cycles of reselection of phage that bind to a Salvador protein and/or a Mutant Salvador protein. Repeated rounds lead to enrichment of phage bearing particular sequences. DNA sequence analysis can be conducted to identify the sequences of the expressed polypeptides. The minimal linear portion of the sequence that binds to the Salvador protein and/or to the Mutant Salvador protein can be determined. One can repeat the procedure using a biased library containing inserts containing part or all of the minimal linear portion plus one or more additional degenerate residues upstream or downstream thereof. Thus, the Salvador proteins of the invention can be used to screen peptide libraries, including phage display libraries, to identify and select peptide binding partners of the Salvador proteins and/or Mutant Salvador proteins of the invention. Such molecules can be used, as described, for screening assays, for diagnostic assays, for purification protocols or for targeting drugs, toxins and/or labeling agents (e.g., radioisotopes, fluorescent molecules, etc.) to cells which express Mutant salvador genes such as cancer cells which have aberrant salvador expression. As detailed herein, the foregoing antibodies and other binding molecules may be used to identify tissues with normal or aberrant expression of a Salvador protein, for example, to identify tissues expressing abnormal levels of a Salvador protein and/or of a Mutant Salvador protein, compared to the expression of these proteins in tissues which express a normal level of these proteins (i.e., levels of Salvador and/or Mutant Salvador protein in tissues which are not characterized by tumor cell growth and/or metastasis or other abnormal cell maturation/differentiation, abnormal cell growth, abnormal cell proliferation, abnormal cell death, and/or abnormal cell apoptosis. Additionally, the binding molecules may be used to purify Salvador proteins or Mutant Salvador proteins.

Antibodies also may be coupled to specific diagnostic labeling agents for imaging of cells and tissues with normal or aberrant salvador expression or to therapeutically useful agents according to standard coupling procedures. Diagnostic agents include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99, iodine-131 and indium-111, and nuclides for nuclear magnetic resonance such as fluorine and gadolinium. Other diagnostic agents useful in the invention will be apparent to one of ordinary skill in the art.

As used herein, “therapeutically useful agents” include any therapeutic molecule known in the art. These agents include antineoplastic agents, radioiodinated compounds, toxins, other cytostatic or cytolytic drugs, and so forth. Antineoplastic therapeutics are well known and include: aminoglutethimide, azathioprine, bleomycin sulfate, busulfan, carmustine, chlorambucil, cisplatin, cyclophosphamide, cyclosporine, cytarabidine, dacarbazine, dactinomycin, daunorubicin, doxorubicin, taxol, etoposide, fluorouracil, interferon, lomustine, mercaptopurine, methotrexate, mitotane, procarbazine HCl, thioguanine, vinblastine sulfate and vincristine sulfate. Additional antineoplastic agents include those disclosed in Chapter 52, Antineoplastic Agents (Paul Calabresi and Bruce A. Chabner), and the introduction thereto, pp. 1202-1263, of Goodman and Gilman's “The Pharmacological Basis of Therapeutics,” Eighth Edition, 1990, McGraw-Hill, Inc. (Health Professions Division). Toxins can be proteins such as, for example, pokeweed anti-viral protein, cholera toxin, pertussis toxin, ricin, gelonin, abrin, diphtheria exotoxin, or Pseudomonas exotoxin. Toxin moieties can also be high energy-emitting radionuclides such as cobalt-60. In one embodiment, these agents may be targeted selectively to a cell or tissue selectively with an aberrant salvador expression.

According to a further aspect of the invention, pharmaceutical compositions containing the nucleic acid molecules, proteins, and binding polypeptides of the invention are provided. The pharmaceutical compositions contain any of the foregoing molecules of the invention in a pharmaceutically acceptable carrier. Thus, in a related aspect, the invention provides a method for forming a medicament that involves placing a therapeutically effective amount of a molecule of the invention in the pharmaceutically acceptable carrier to form one or more doses.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

The therapeutics of the invention can be administered by any conventional route, including injection or by gradual infusion over time. The administration may, for example, be oral, intravenous, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712). Those of skill in the art can readily determine the various parameters and conditions for producing antibody aerosols without undue experimentation.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, and lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases, and the like.

The preparations of the invention are administered in effective amounts. As used herein, an “effective amount” is that amount of a pharmaceutical preparation that alone, or together with further doses, stimulates a desired response. In the case of treating cancer, the desired response is inhibiting the progression of the cancer. This may involve only slowing the progression of the disease temporarily, although more preferably, it involves halting the progression of the disease permanently. In the case of stimulating an immune response (e.g., for research applications to prepare antibodies or for therapeutic applications to induce antibodies to a Mutant salvador molecule but not to a salvador molecule), the desired response is an increase in antibodies or T lymphocytes which are specific for the immunogen(s) employed. These responses can be monitored by routine methods or can be monitored according to diagnostic methods of the invention discussed herein.

Where it is desired to stimulate an immune response using a therapeutic composition of the invention (e.g., a Mutant Salvador protein fragment which is a unique fragment of the Mutant salvador molecule and is absent from the Salvador protein), a desired response may involve the stimulation of a humoral antibody response resulting in an increase in antibody titer in serum, a clonal expansion of cytotoxic lymphocytes, or some other desirable immunologic response. It is believed that doses of immunogens ranging from one nanogram/kilogram to 100 milligrams/kilogram, depending upon the mode of administration, would be effective. The preferred range is believed to be between 500 nanograms and 500 micrograms per kilogram. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

According to another aspect of the invention, various diagnostic methods are provided. In general, the methods are for diagnosing a disorder characterized by aberrant expression of a salvador molecule or a Mutant salvador molecule. As used herein, “aberrant expression” refers to either or both of a decreased expression (including no detectable expression) of a Salvador molecule (nucleic acid or protein) or an increased expression of a “Mutant salvador molecule”. A Mutant Salvador molecule refers to a Salvador nucleic acid molecule which includes a mutation (point, deletion, addition, substitution, rearrangement, truncation) or to a Mutant Salvador protein molecule (e.g., gene product of Mutant Salvador nucleic acid molecule) which includes a mutation, provided that the mutation results in a Mutant Salvador protein that does not have a Salvador protein functional activity. The diagnostic methods of the invention can be used to detect the presence of a disorder associated with aberrant expression of a Salvador molecule or a Mutant salvador molecule, as well as to assess the progression and/or regression of the disorder such as in response to treatment (e.g., chemotherapy, radiation).

According to this aspect of the invention, the method for diagnosing a disorder characterized by aberrant expression of a Salvador molecule or a Mutant salvador molecule involves: detecting in a first biological sample obtained from a subject, expression of a salvador molecule or a Mutant salvador molecule; wherein decreased expression of a salvador molecule or increased expression of a Mutant salvador molecule compared to a control sample indicates that the subject has a disorder characterized by aberrant expression of a salvador molecule.

In one embodiment, a probe sequence is used to screen for aberrant expression of a salvador molecule. In a preferred embodiment, the probe has the sequence set forth as SEQ ID NO:5, and is used to screen human cells for aberrant, including no detectable expression, of the salvador nucleic acid.

As used herein, a “disorder characterized by aberrant expression of a salvador molecule” refers to a disorder in which there is a detectable difference in the expression levels of salvador molecule(s) and/or Mutant salvador molecule(s) in selected cells of a subject compared to the control levels of these molecules in the cells obtained from subjects who do not exhibit tumor growth, metastasis, abnormal cellular development, abnormal cell proliferation, abnormal cell death, and/or abnormal apoptosis. Thus, a disorder characterized by aberrant expression of a salvador molecule embraces reduced expression (including no detectable expression) of a salvador nucleic acid molecule or a Salvador protein compared to control levels of these molecules, as well as enhanced expression of a Mutant salvador nucleic acid molecule or Mutant Salvador protein compared to control levels of these molecules. Such differences in expression levels can be determined in accordance with the diagnostic methods of the invention as disclosed herein. Exemplary disorders that are characterized by aberrant expression of a Salvador molecule include: various cancers, birth defects, and autoimmunity disorders. In general, each of these disorders is associated with abnormal cell proliferation, abnormal cell death, abnormal cellular growth and abnormal cell maturation/differentiation, and/or abnormal cell-cell interactions which, for example, characterize tumor cell growth and metastasis.

In certain embodiments, the methods of the invention are to diagnose a cancer including, but not limited to, kidney cancer, biliary tract cancer, brain cancer (including glioblastomas and medulloblastomas), breast cancer, cervical cancer, choriocarcinoma, colon cancer, endometrial cancer, esophageal cancer, gastric cancer, hematological neoplasms, including acute lymphocytic and myelogenous leukemia, multiple myeloma, AIDS associated leukemias and adult T-cell leukemia lymphoma, intraepithelial neoplasms, including Bowen's disease and Paget's disease, liver cancer, lung cancer, lymphomas, including Hodgkin's disease and lymphocytic lymphomas, neuroblastomas, oral cancer, including squamous cell carcinoma, ovarian cancer, including those arising from epithelial cells, stromal cells, germ cells and mesenchymal cells, pancreatic cancer, prostate cancer, rectal cancer, renal cancer including adenocarcinoma and Wilms tumor, sarcomas, including leiomyosarcoma, rhabdomyosarcoma, liposarcoma, fibrosarcoma and osteosarcoma, skin cancer, including melanoma, Kaposi's sarcoma, basocellular cancer, squamous cell cancer, testicular cancer, including germinal tumors (seminoma, non-seminoma [teratomas, choriocarcinomas]), stromal tumors and germ cell tumors, and thyroid cancer, including thyroid adenocarcinoma and medullary carcinoma. In the preferred embodiments, the methods of the invention are useful for diagnosing liver cancer, ovarian cancer, testicular cancer, and biliary tract cancer.

In certain embodiments, the methods of the invention are to diagnose a birth defect including, but not limited to, defects resulting from increased or decreased cell proliferation, benign and malignant childhood tumors, neural tube defects, mental retardation, congenital malformations resulting from the absence of normal structure, and supernumerary structures, for example, additional digits.

The method may be used on biological samples including, but not limited to, amniotic fluid samples, chorionic villus biopsy samples and cell-containing samples from preimplantation embryos, particularly cell-containing samples from early preimplantation embryos stage (cell numbers equal to or less than 64). Therefore, the invention also contemplates prenatal diagnosis of a disorder.

In yet other embodiments, the methods of the invention are useful for diagnosing an autoimmunity disorder, including, but not limited to, rheumatoid arthritis, multiple sclerosis, type 1 diabetes, psoriasis, and inflammatory bowel disease (IBD).

In the foregoing embodiments, the biological sample can be any nucleic acid- or protein-containing sample obtained from a subject. Exemplary biological samples are described below. In yet other embodiments, the diagnostic methods are useful for diagnosing the progression of a disorder. According to these embodiments, the methods further involve: detecting in a second biological sample obtained from the subject, expression of a salvador molecule or a Mutant salvador molecule, and comparing the expression of the salvador molecule or the Mutant salvador molecule in the first biological sample and the second biological sample. In these embodiments, a decrease in the expression of the salvador molecule in the second biological sample compared to the first biological sample or an increase in the expression of the Mutant salvador molecule in the second biological sample compared to the first biological sample indicates progression of the disorder.

In yet other embodiments, the diagnostic methods are useful for diagnosing the regression of a disorder. According to these embodiments, the methods further involve: detecting in a second biological sample obtained from the subject, expression of a salvador molecule or a Mutant salvador molecule, and comparing the expression of the salvador molecule or the Mutant salvador molecule in the first biological sample and the second biological sample. In these embodiments, an increase in the expression of the salvador molecule in the second biological sample compared to the first biological sample or a decrease in the expression of the Mutant salvador molecule in the second biological sample compared to the first biological sample indicates regression of the disorder.

In certain embodiments, the diagnostic methods of the invention detect a salvador molecule that is a salvador nucleic acid molecule or a Mutant salvador nucleic acid molecule as described above. In yet other embodiments, the methods involve detecting a Salvador protein or Mutant Salvador protein as described above.

Various detection methods can be used to practice the diagnostic methods of the invention. For example, the methods can involve contacting the biological sample with an agent that selectively binds to the salvador molecule or to the Mutant salvador molecule to detect these molecules. In certain embodiments, the salvador molecule or the Mutant salvador molecule is a nucleic acid and the method involves using an agent that selectively binds to the salvador molecule or to the Mutant salvador molecule, e.g., a nucleic acid that hybridizes to SEQ ID NO:1 or to SEQ ID NO:3 or to SEQ ID NO:6 under stringent conditions. In yet other embodiments, the salvador molecule or the Mutant salvador molecule is a protein and the method involves using an agent that selectively binds to the Salvador protein or to the Mutant Salvador protein, e.g., a binding polypeptide, such as an antibody, that selectively binds to SEQ ID NO:2 or to SEQ ID NO:4.

According to still another aspect of the invention, kits for performing the diagnostic methods of the invention are provided. In general, the kits are nucleic acid-based kits or protein-based kits. According to the former embodiment, the kits include: one or more nucleic acid molecules that hybridize to a salvador nucleic acid molecule (cDNA or genomic sequence) or to a Mutant salvador nucleic acid molecule (cDNA or genomic sequence) under stringent conditions; one or more control agents; and instructions for the use of the nucleic acid molecules, and agents in the diagnosis of a disorder associated with aberrant expression of a salvador molecule. Nucleic acid-based kits optionally further include a first primer and a second primer, wherein the first primer and the second primer are constructed and arranged to selectively amplify at least a portion of an isolated salvador nucleic acid molecule comprising SEQ ID NO:1 or SEQ ID NO:6. Alternatively, the kits include two isolated nucleic acid molecules, the first consisting of a 20-32 nucleotide contiguous segment of SEQ ID NO:1 or SEQ ID NO:6 and the second consisting of a 20-32 nucleotide contiguous segment of the complement of SEQ ID NO:1 or SEQ ID NO:6 that does not overlap the first segment. Optionally the isolated nucleic acids are unique fragments of SEQ ID NO:1 or SEQ ID NO:6 or the complements of SEQ ID NO:1 or SEQ ID NO:6. The first and second isolated nucleic acid molecules are designed to act as primers capable of selectively amplifying at least a portion or all of SEQ ID NO:1 or SEQ ID NO:6 and are nonoverlapping to prevent the formation of primer-dimers. One of the primers will hybridize to one strand of the nucleic acid to be amplified and the second primer will hybridize to the complementary strand of the nucleic acid to be amplified, in an arrangement which permits amplification of the nucleic acid. Selection of appropriate primer pairs is standard in the art. For example, the selection can be made with assistance of a computer program designed for such a purpose, optionally is followed by testing the primers for amplification specificity and efficiency.

Alternatively, protein based-kits are provided. Such kits include: one or more binding polypeptides that selectively bind to a Salvador protein or to a Mutant Salvador protein; one or more control agents; and instructions for the use of the binding polypeptides, and agents in the diagnosis of a disorder associated with aberrant expression of a salvador molecule. In the preferred embodiments, the binding polypeptides are antibodies or antigen-binding fragments thereof, such as those described above. In these and other embodiments, certain of the binding polypeptides bind to the Mutant Salvador protein but do not bind to the Salvador protein to further distinguish the expression of these proteins in a biological sample.

As used herein, the term “subject” is used to describe a human, non-human primate, cow, horse, pig, sheep, goat, dog, cat, rodent, insect such as Drosophila, or nematode such as C. elegans. In all embodiments human and Drosophila salvador molecules and human subjects are preferred.

The biological sample can be located in vivo or in vitro. For example, the biological sample can be a tissue in vivo and the agent specific for the tumor associated nucleic acid molecule or polypeptide can be used to detect the presence of such molecules in kidney tissue (e.g., for imaging portions of the tissue that express the tumor associated gene products). Alternatively, the biological sample can be located in vitro (e.g., a blood sample, tumor biopsy, tissue extract). In a particularly preferred embodiment, the biological sample can be a cell-containing sample, more preferably a sample containing tumor cells. Samples of tissue and/or cells for use in the various methods described herein can be obtained through standard methods. Samples can be surgical samples of any type of tissue or body fluid. Samples can be used directly or processed to facilitate analysis (e.g., by parafin embedding). Exemplary samples include a cell, a cell scraping, a cell extract, a blood sample, a tissue biopsy, including punch biopsy, a tumor biopsy, cells from a preimplantation embryo, a bodily fluid, a tissue, or a tissue extract or other methods.

The invention also provides treatment methods. As used herein, “treatment” includes preventing, delaying, abating or arresting the clinical symptoms of a disorder characterized by aberrant expression of a salvador molecule. Thus, treatment includes reducing or preventing tumor cell growth, proliferation, and/or metastasis, as well as reducing or preventing any manifestation of abnormal cell maturation/differentiation, cell growth, proliferation, or death (including apoptosis).

In general, the treatment methods involve administering an agent to increase expression of a salvador molecule and/or reduce expression of a Mutant salvador molecule. Thus, these methods include gene therapy applications. In certain embodiments, the method for treating a subject with a disorder characterized by aberrant expression of a salvador molecule, involves administering to the subject an effective amount of a salvador nucleic acid molecule to treat the disorder. An exemplary molecule for inhibiting expression of a Mutant salvador nucleic acid molecule is an anti-sense molecule that is selective for the mutant nucleic acid and that does not inhibit expression of the salvador nucleic acid molecule. Alternatively, the method for treating a subject with a disorder characterized by aberrant expression of a Salvador molecule involves administering to the subject an effective amount of a Salvador protein to treat the disorder. In yet another embodiment, the treatment method involves administering to the subject an effective amount of a binding polypeptide to inhibit a Mutant Salvador protein and, thereby, treat the disorder. In certain preferred embodiments, the binding polypeptide is an antibody or an antigen-binding fragment thereof; more preferably, the antibodies or antigen-binding fragments are labeled with one or more cytotoxic agents

The invention also contemplates gene therapy. The procedure for performing ex vivo gene therapy is outlined in U.S. Pat. No. 5,399,346 and in exhibits submitted in the file history of that patent, all of which are publicly available documents. In general, it involves introduction in vitro of a functional copy of a gene into a cell(s) of a subject which contains a defective copy of the gene, and returning the genetically engineered cell(s) to the subject. Alternatively, a functional copy of the gene may be introduced into a cell that is missing a functional copy of the gene. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo gene therapy using vectors such as adenovirus, retroviruses, herpes virus, and targeted liposomes also is contemplated according to the invention.

In preferred embodiments, a virus vector for delivering a nucleic acid molecule encoding a Salvador protein is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia-virus derivative (Paoletti, Proc. Natl. Acad Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol 26:1951-1959, 1996). In preferred embodiments, the virus vector is an adenovirus.

Another preferred virus for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Cliffton, N.J. (1991).

Preferably, the foregoing nucleic acid delivery vectors: (1) contain exogenous genetic material that can be transcribed and translated in a mammalian cell and that can suppress tumor cell growth and/or proliferation and/or metastasis, and/or other abnormal cell maturation/differentiation, cell growth, cell proliferation, cell death, cell migration, and/or cell-cell interaction in a host, and preferably (2) contain on a surface a ligand that selectively binds to a receptor on the surface of a target cell, such as a mammalian cell, and thereby gains entry to the target cell.

Various techniques may be employed for introducing nucleic acid molecules of the invention into cells, depending on whether the nucleic acid molecules are introduced in vitro or in vivo in a host. Such techniques include transfection of nucleic acid molecule-CaPO₄ precipitates, transfection of nucleic acid molecules associated with DEAE, transfection or infection with the foregoing viruses including the nucleic acid molecule of interest, liposome mediated transfection, and the like. For certain uses, it is preferred to target the nucleic acid molecule to particular cells. In such instances, a vehicle used for delivering a nucleic acid molecule of the invention into a cell (e.g., a virus such as a retrovirus, or a liposome) can have a targeting molecule attached thereto. For example, a molecule such as an antibody specific for a surface membrane protein on the target cell or a ligand for a receptor on a target cell can be bound to or incorporated within the nucleic acid molecule delivery vehicle. Especially preferred are monoclonal antibodies. Where liposomes are employed to deliver the nucleic acid molecules of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acid molecules into cells, as is known by those skilled in the art. Such systems even permit oral delivery of nucleic acid molecules.

The invention provides various research methods and compositions. Thus, according to one aspect of the invention, a method for producing a Salvador protein is provided. The method involves providing a salvador nucleic acid molecule operably linked to a promoter, wherein the salvador nucleic acid molecule encodes the Salvador protein or a fragment thereof; expressing the salvador nucleic acid molecule in an expression system; and isolating the Salvador protein or a fragment thereof from the expression system. Preferably, the salvador nucleic acid molecule has SEQ ID NO:1 or SEQ ID NO:3. According to yet another aspect of the invention, a method for producing a Mutant Salvador protein is provided. This method involves: providing a Mutant salvador nucleic acid molecule operably linked to a promoter, wherein the Mutant salvador nucleic acid molecule encodes the Mutant Salvador protein or a fragment thereof; expressing the Mutant salvador nucleic acid molecule in an expression system; and isolating the Mutant Salvador protein or a fragment thereof from the expression system. Preferably, the Mutant salvador nucleic acid molecule has SEQ ID NO:1 or SEQ ID NO:3 with one or more deletions, additions, rearrangements, substitutions, or truncations to encode a Mutant Salvador protein.

The invention further provides efficient methods of identifying pharmacological agents or lead compounds for agents which mimic the functional activity of a salvador molecule. Generally, the screening methods involve assaying for compounds which modulate (i.e., up- or down-regulate) a salvador functional activity. For example, a genetic screen can be used to identify salvador molecules that function in G1 to facilitate or inhibit cell cycle entry. This approach stems from our observation that the human cyclin-dependent kinase inhibitor p21, when expressed in the developing Drosophila eye, can function to arrest cell cycle progression of precursor cells and results in the development of a rough eye due to a deficit of retinal cells. This rough eye phenotype is extremely sensitive to levels of endogenous G1 regulators and interacts with mutations in DmcycE (cyclin E), dE2, and roughex, but not with mutations in cell cycle regulators that function at other stages of the cell cycle. Thus, flies overexpressing p21 can be used to identify additional salvador molecules and Mutant salvador molecules that regulate entry into or exit from the cell cycle. (See, e.g., the Examples).

A wide variety of assays for pharmacological agents can be used in accordance with this aspect of the invention, including, labeled in vitro protein-protein binding assays, electrophoretic mobility shift assays, immunoassays, cell-based assays such as two- or three-hybrid screens, expression assays, apoptosis assays, etc. An assay mixture comprises a candidate pharmacological agent. Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection. Candidate agents encompass numerous chemical classes, although typically they are organic compounds. Preferably, the candidate pharmacological agents are small organic compounds, i.e., those having a molecular weight of more than 50 and less than about 2500, preferably less than about 1000 and, more preferably, less than about 500. Candidate agents comprise functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc., which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease is inhibitors, antimicrobial agents, and the like may also be used.

An exemplary binding assay is described herein. In general the mixture of the foregoing assay materials is incubated under conditions whereby, but for the presence of the candidate pharmacological agent, the salvador molecule or the Mutant salvador molecule specifically binds the binding agent (e.g., antibody, complementary nucleic acid). The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the presence or absence of specific binding between the salvador molecule or the Mutant salvador molecule and one or more binding agents is detected by any convenient method available to the user. For cell free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximum signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromotograpic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. When the solid substrate is a magnetic bead, the beads may be washed one or more times with a washing solution and isolated using a magnet.

Detection may be effected in any convenient way for cell-based assays such as two- or three-hybrid screens. For cell free binding assays, one of the components usually comprises, or is coupled to, a detectable label. A wide variety of labels can be used, such as those that provide direct detection (e.g., radioactivity, luminescence, optical or electron density, etc.) or indirect detection (e.g., epitope tag such as the FLAG epitope, enzyme tag such as horseradish peroxidase, etc.). The label may be bound to a salvador binding partner (e.g., polypeptide), or incorporated into the structure of the binding partner.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradioactive energy transfers, etc., or indirectly detected with antibody conjugates, strepavidin-biotin conjugates, etc. Methods for detecting the labels are well known in the art.

According to yet another aspect of the invention, a method for identifying a salvador molecule or a Mutant salvador molecule is provided. In certain embodiments, the putative salvador molecule is derived from a human cell, a Drosophila, or a nematode (e.g., C. elegans). The method for identifying a salvador molecule or a Mutant salvador molecule involves: (a) introducing a putative salvador molecule or a Mutant salvador molecule into a cell; and (b) detecting a salvador functional activity. The salvador functional activity is selected from the group of activities consisting of binding to a cognate molecule (e.g., a warts/LATS molecule) containing a PPPY motif which binds to the WW domain of a Salvador protein, modulating cell maturation/differentiation, modulating cell growth, modulating cell proliferation, and modulating cell death.

Warts/LATS was originally identified in Drosophila and refers to the same gene having different names assigned by different laboratories. LATS has GenBank accession number U29608 (SEQ ID NO: 18) for the Drosophila melanogaster large tumor suppressor (lats) mRNA, long transcript, complete cds; warts has GenBank accession number L39837 (SEQ ID NO: 19) for the Drosophila melanogaster tumor suppressor (warts) mRNA exons 1-8, complete cds. The PPPY motif in the warts LATS protein is at position 541-544 of SEQ ID NO: 18 and SEQ ID NO: 20. As used herein, a warts/LATS molecule refers to a nucleic acid or protein molecule that comprises a portion of warts/LATS molecule containing the PPPY motif (or which encodes this motif). Preferably, the warts/LATS molecule contains a sufficient number of amino acids on either side of the PPPY motif to retain sufficient secondary and, optionally, tertiary structure in the molecule to allow binding of the PPPY motif to a Salvador protein. In general, the warts/LATS molecule contains at least from about 1 to about 50 amino acids (and every integer therebetween) from the natural warts/LATS protein on either or both of the 5′ and 3′ terminal ends of the PPPY motif. The invention also provides nucleic acids which encode these portions of the naturally occurring warts/LATS molecule. Thus, these portions of the naturally occurring warts/LATS molecules retain their ability to selectively bind to a Salvador protein and can be used, for example, in binding assays to identify agents which modulate this interaction and to further define the pathways involving this interaction.

The human warts/LATS homologs, LATS1 and LATS2, have GenBank accession numbers AF104413 (SEQ ID NO: 21) and AB028019 (SEQ ID NO:23), respectively; although the cDNA sequence for LATS2 is incomplete. Like the Drosophila warts/LATS gene, the human homolog contains a PPPY motif which is believed to bind to a WW domain in the human Salvador protein.

According to still another aspect of the invention, a method for identifying a salvador modulating agent that modulates a salvador molecule-cognate interaction is provided. As used herein, a “cognate” is a molecule to which a salvador molecule binds. The method involves: (a) contacting a salvador molecule with a cognate, in the presence of a putative modulating agent, under conditions to allow the salvador molecule to bind to the cognate (e.g., warts/LATS); and (b) detecting salvador molecule binding to the cognate. A change in salvador molecule binding to the cognate in the presence of the putative modulating agent compared to salvador molecule binding to the cognate in the absence of the cognate indicates that the agent is a salvador modulating agent, that is, a molecule that affects the binding properties of a salvador molecule and a cognate. In general, detecting comprises detecting a change in a parameter selected from salvador molecule binding to its cognate, cell maturation/differentiation, cell growth, cell proliferation, and/or cell death.

The invention will be more fully understood by reference to the following examples. These examples, however, are merely intended to illustrate the embodiments of the invention and are not to be construed to limit the scope of the invention.

### EXAMPLES

### Example 1

### Introduction to Identification of Salvador Molecules

Mutations in the salvador gene in Drosophila were identified by detecting mutant cells having a proliferative or growth advantage over wild type cells using FLP-induced mitotic recombination in the eye (described in detail below and in Tapon, N., et al., Cell, 110(4):467, 2002, hereby incorporated by reference).

More than 200 mutations were found using the above-identified assay. These mutations include the Mutant salvador nucleic acid molecules having SEQ ID NOs: 15, 17, and 19; and their gene products having SEQ ID NOs: 16, 18, and 20.

Clones of the mutant Drosophila cells were generated in wing and notum tissues and resulted in the appearance of tumor-like growths on this tissues. Further characterization of the clones included visualizing cell proliferation using BrdU incorporation and TUNEL labeling to visualize cell death. The results of these studies indicated that the mutant Drosophila cells do not arrest in G1 phase and continue to S-phase and that there is almost no apoptosis in mutant clones.

Meiotic mapping of the Drosophila salvador gene identified CG13832 as the genomic sequence which includes an open reading frame corresponding to the cDNA for the Drosophila salvador gene having SEQ ID NO:3 and its protein product having SEQ ID NO:4. Mutant Drosophila salvador nucleic acid molecules were sequenced in accordance with standard procedures. The Mutant Drosophila salvador molecules of the invention that have been identified in accordance with this procedure include Mutant salvador nucleic acid molecules (SEQ ID NOs:15, 17, and 19) and Mutant Salvador proteins (SEQ ID NOs:16, 18, and 20).

The human homologue to the Drosophila salvador gene was identified as the hWW45 gene (GenBank accession number NM0218118) reported by Valverde (Biochem. Biophys. Res. Commun. 276 990-998, 2000), with the human salvador cDNA having SEQ ID NO:1 and the encoded human Salvador protein having SEQ ID NO:2.

Screening for the salvador gene in human cancer cells lines was accomplished using a probe (SEQ ID NO:5) for the human salvador gene, in accordance with the procedures described below.

The results obtained using SEQ ID NO:5 as the hybridization probe established that all coding sequences of the human salvador gene are deleted in the ACHN human kidney tumor cell lines and exons 3, 4, and 5 (nucleotides 25027-25297, nucleotides 27622-27765, and nucleotides 33131-34873, of Seq ID NO:6, respectively) are deleted in the 786-0 human kidney tumor cell line.

Mutant human salvador molecules are identified in the accordance with the above-described procedures and are sequenced in accordance with standard procedures. In general, the Mutant salvador nucleic acid molecules contain a sequence which is the same as SEQ ID NO:1 or SEQ ID NO:3, with the exception that the sequence includes one or more mutations, e.g., point mutations, deletion mutations, or truncations, such that the Mutant salvador nucleic acid molecule does not encode a functional Salvador protein. Rather, the Mutant salvador nucleic acid molecules encode a Mutant Salvador protein, i.e., a protein which does not exhibit Salvador protein functional activity. In certain preferred embodiments, the Mutant human salvador nucleic acid molecules are truncated forms of SEQ ID NO: 1 which lack one or more WW domains. The WW domains are located at positions 813-911 and 918-1016 of SEQ ID NO:1. In related embodiments, the Mutant human Salvador proteins are truncated forms of SEQ ID NO:2 which lack one or more WW domains. The WW domains in the protein sequence are located at positions 200-232 and 235-267 of SEQ ID NO:2.

### Example 2

### Screening Methods and Results

A genetic screen in Drosophila was used to identify genes that restrict growth and proliferation. Once the Drosophila salvador gene was identified, database searches were performed to identify the human homologue. Once identified, tumor cell lines were examined for mutations in the putative tumor suppressor gene.

In order to identify genes that restrict cell numbers in vivo, a genetic screen in Drosophila was conducted to identify mutations in genes that result in mutant cells having even a subtle growth or proliferative advantage over their wild-type neighbors. Using FLP-induced mitotic recombination in the eye, clones of mutant tissue were generated and compared in size to the wild-type twin spots generated from the same recombination event. After screening the four major autosomal arms which together account for approximately 80% of the genome, more than 200 mutations that result in this phenotype were identified. Of these, 23 loci are represented by more than a single allele. These include mutations in Drosophila homologues of known human tumor suppressor genes including PTEN (5 alleles) and the Tuberous Sclerosis Complex (Tsc1, 3 alleles; Tsc2, 2 alleles).

Another locus identified in the screen, represented by 3 alleles is a novel gene salvador which corresponds to the transcription unit designated CG13832. Mutations in salvador result in inappropriate cell proliferation and a failure of cell cycle exit. Moreover, mutant cells appear to be resistant to some of the apoptotic signals that result in the death of is their wild-type neighbors. Clones of mutant salvador cells can give rise to tumorous outgrowths. We have cloned the salvador gene. It encodes a novel protein with two WW-domains with highly conserved orthologues in C. elegans and mammals. The human homologue of salvador, hWW45 (accession number NM021818), containing two WW-domains from amino acids 200 to 232 and 235 to 267, has been identified previously but its function was not known. We have now shown that hWW45 appears to be deleted or rearranged in at least two human kidney cancer cell lines.

To identify genes that restrict cell growth or cell numbers in vivo, a screen in the Drosophila eye for mutations that increased the relative representation of mutant tissue compared to wild-type tissue was conducted (Tapon, N., et al. (2001) Cell 105, 345-355). Using FLP/FRT-induced mitotic recombination, clones of white mutant tissue were compared in size to sister clones of red wild-type tissue. Those flies whose eyes contained an excess of mutant over wild-type tissue were retained. Mutations in at least 23 distinct loci that elicit this phenotype were identified. These included negative regulators of cell proliferation such as archipelago (ago) as well as homologs of human tumor-suppressor genes including PTEN, TSC1, and TSC2 (Moberg, K. H., et al. (2001) Nature 413, 311-316; Tapon, N., et al. (2001) Cell 105, 345-355).

Three alleles of salvador were identified using this screen. A fourth allele, salvador⁴, was isolated by Jessica Treisman and kindly provided. Salvador¹ and salvador² generated eyes that have an increased representation of mutant tissue over wild-type tissue when compared to the parent chromosome. Salvador³ elicited a more severe phenotype; in addition to a further increase in the representation of mutant tissue, the mutant tissue protruded from the eye in folds. Salvador⁴ exhibited an intermediate phenotype. Clones of salvador³ mutant s tissue generated in other parts of the fly including the notum and haltere also displayed outgrowths. All four alleles were lethal when homozygous, in trans to each other or in trans to the deletion Df(3R)hh that spans the salvador locus.

In salvador¹ clones in the adult retina, almost all the ommatidia contained the normal complement of eight photoreceptor cells. However, there was increased spacing between adjacent ommatidia. In contrast to wild-type retinas from late pupae that contained a single layer of interommatidial cells, mutant clones contained many additional interommatidial cells. Generation of salvador¹ mutant clones in a white⁺ background indicated that most of these additional interommatidial cells contained pigment. Thus, these cells underwent terminal differentiation. The more disorganized retinas of the salvador³ allele displayed all of these phenotypic abnormalities. In addition, almost half of the ommatidia in salvador³ clones lacked one or more photoreceptor cells.

### Example 3

### Salvador Promotes Cell Cycle Exit

In wild-type imaginal discs, S phases, as visualized by BrdU incorporation, were observed anterior to the morphogenetic furrow (MF) and as a single stripe of incorporation posterior to the furrow referred to as the second mitotic wave (SMW). In salvador clones, many BrdU-incorporating nuclei were observed posterior to the SMW. Clones spanning the MF had some BrdU-incorporating nuclei in the anterior half of the MF, a region that is normally composed of cells arrested in G1. Using the anti-phosphohistone H3 antibody, additional cells in mitosis were also visualized in salvador mutant clones posterior to the MF, suggesting that at least some of these cells had completed additional cell cycles. BrdU incorporation persisted in mutant clones during the first 12 hr after puparium formation (APF) but ceased by 24 hr APF. Thus, salvador mutant cells continued to proliferate for 12 to 24 hr after wild-type cells stopped dividing but eventually were able to exit from the cell cycle to undergo terminal differentiation.

In cycling cells in the anterior portion of the eye imaginal disc, the distribution of mutant cells in the cell cycle, as assessed by flow cytometry, was extremely similar to that of wild-type cells. The mutant cells were very slightly smaller than their wild-type counterparts. Posterior to the MF, mutant populations had an increased proportion of cells in S and G2, indicating that mutant cells continued to cycle in this portion of the disc. Mutant cells were of normal size. The population doubling times of clones of mutant cells and wild-type cells generated in the wing imaginal disc during the proliferative phase of development did not differ significantly. Thus, when proliferating, mutant cells behaved like wild-type cells. However, exit from the cell cycle was delayed in salvador cells.

Elevated levels of Cyclin E protein were found in the basal nuclei of salvador clones posterior to the MF. These were the nuclei of the undifferentiated cells that continued to proliferate in salvador clones. They were examined for levels of cyclin E RNA. When salvador clones were generated using eyFLP (Newsome, T. P., et al. (2000) Development 127, 851-860), a large proportion of cells in third instar discs were mutant, and these discs contained large patches of mutant tissue.

In wild-type discs, cyclin E RNA was expressed in a narrow stripe immediately posterior to the morphogenetic furrow. In discs containing salvador clones, the stripe of expression was broader and more intense, indicating that cyclin E RNA levels were elevated in these discs. Thus, the increased level of Cyclin E protein is likely to have resulted from, at least in part, from an increase in cyclin E RNA levels.

### Example 4

### Salvador is Required for Apoptosis in the Eye Imaginal Disc

In wild-type eyes, excessive interommatidial cells are eliminated by a wave of apoptosis that is evident in 38 hr pupal retinas (Wolff, T. and Ready, D. F. (1993) Pattern formation in the Drosophila retina. In The Development of Drosophila melanogaster, M. Bate, and A. Martinez Arias, eds. (Plainview, New York: Cold Spring Harbor Laboratory Press) 1277-1325). Even in salvador mutant clones, cell proliferation, as assessed by BrdU incorporation, had ceased within 24 hr APF. When mosaic retinas were examined 38 hr APF, cell death was mostly confined to the wild-type portions of the retina. Thus, the apoptotic cell deaths that are part of normal retinal development appear to require salvador function.

Apoptosis in the pupal retina requires hid function, since hid mutants display additional interommatidial cells (Kurada, P. and White K. (1998) Cell 95, 319-329). Hid is thought to induce caspase activation by binding to the DIAP1 protein and preventing it from inhibiting caspase function (Goyal, L., et al. (2000) EMBO J. 19, 589-597; Lisi, S., et al. (2000) Genetics 154, 669-678; Wang, S. L. (1999) Cell 98, 453463). Overexpression of hid using the eye-specific GMR promoter generated a small eye (Hay, B. A., et al. (1995) Cell 83, 1253-1262). The induction of cell death by hid was severely impaired in salvador mutant clones. As a consequence, eyes derived from GMR-hid-expressing discs that contain salvador mutant clones were larger than those derived from wild-type discs that express GMR-hid. Since salvador function is required for hid-induced cell death, salvador is believed to function either downstream of hid or in a parallel pathway.

Several recent studies have shown that another mechanism by which Hid and Rpr activate caspases is by inducing the autoubiquitination of DIAP1 and targeting it for degradation by the proteasome (Hays, R., et al. (2002) Nat. Cell Biol. 4, 425431; Holley, C. L., et al. (2002) Nat. Cell Biol, 4, 439444; Ryoo, H. D., et al. (2002) Nat. Cell Biol. 4, 432-438; Wilson, R., et al. (2002) Nat. Cell Biol. 4, 445-450; Wing, J. P., et al. (2002) Nat. Cell Biol. 4, 451456; and Yoo, S. J., et al. (2002) Nat. Cell Biol. 4, 416424). It was observed that DIAP1 levels were markedly elevated in salvador clones in the larval eye disc, and remained elevated in the interommatidial cells in mutant clones in the pupal eye disc, where a reduction of apoptosis was observed. Thus, it is believed that increased levels of DIAP1 in salvador cells may be able to overcome the effect of many proapoptotic signals.

To examine DIAP1 RNA levels, in situ hybridization was used to examine 20 wild-type discs and 20 mutant discs. The presence of salvador (GFP⁻) clones in the mutant discs was confirmed by examining the discs by fluorescence microscopy prior to hybridization. There was a modest level of DIAP1 RNA expression posterior to the furrow in both populations of discs and no evidence of increased DIAP1 RNA in the discs containing salvador clones. Thus, it is believed that this level of detection, the increased DIAP1 expression in salvador cells does not result from increased transcription.

In wild-type eye discs, DIAP1 protein was expressed at higher levels posterior to the morphogenetic furrow. DIAP1 protein levels were downregulated by GMR-rpr or, to a lesser extent, by GMR-hid expression. In salvador mutant clones expressing GMR-rpr, DIAP1 protein levels remained elevated. Similar results were observed with GMR-hid. Thus, it is believed that neither GMR-rpr nor GMR-hid is capable of downregulating the elevated levels of DIAP1 sufficiently in salvador clones to activate caspases.

Expression of hid or reaper (rpr) in the eye imaginal disc resulted in activation of the effector caspase Drice. An antibody that recognizes the cleaved (activated) form of Drice (Yoo, S. J., et al. (2002) Nat. Cell Biol. 4, 416-424) was used to stain eye discs expressing GMR-hid or GMR-rpr. In wild-type cells, Drice was activated by GMR-hid or GMR-rpr. However, in clones of salvador tissue, Drice activation by either GMR-hid or GMR-rpr was almost completely blocked. To counteract the possibility of convolutions in the disc, stainings which were projections of confocal Z series (12 individual frames) spanning the entire thickness of the eye disc, excluding the peripodial membranes, were analyzed. At least 30 discs per genotype from three independent experiments were carefully examined to confirm the results. These experiments indicated that Salvador blocks activation of Drice by both rpr and hid.

A mutant form of Hid (Hid-Ala5) is resistant to inactivation by MAP kinase phosphorylation (Bergmann, A., et al. (1998) Cell 95, 331-341). GMR-hid-Ala5 was a more potent inducer of cell death, as assessed by the extent of Drice activation in the eye disc, than is GMR-hid. Cell death induced by GMR-hid-Ala5 was only partially blocked in salvador clones, indicating that the increased potency of Hid-Ala-5 may have been able to overcome increased DIAP1 levels.

### Example 5

### Salvador Encodes a Protein with WW Domains and has a Human Ortholog

The salvador mutations were localized to the interval 93F11-13 to 94D10-13 (formerly annotated 94D 4-7 to 94E 1-2). High-resolution meiotic mapping localized salvador to a 20 kb region that contained five ORFs. All five ORFs were sequenced completely and it was found that all four Salvador chromosomes had truncating mutations in CG13831. The other four ORFs did not have any amino acid changes.

Meiotic mapping using multiplying marked chromosomes, and subsequently using deletions, allowed for localization of Salvador to the region 94D 4-7 to 94E 1-2 on the cytogenetic map. Salvador was crossed to all the lethal P-element insertions in the region and all of them were able to complement Salvador. Thus the cloning of salvador was not facilitated by the finding of a P-element insertion in the gene. Recombination in males was then initiated by the mobilization of a P-element to locate salvador with respect to identified P-element insertion. It was determined that salvador maps to the right of a P-element in the region of the klingon gene and left of a P-element in hedgehog. A recombinant chromosome was generated that had the klingon insertion in cis to salvador. The chromosome containing the P-element insertion in hedgehog was placed in trans and recombinant chromosomes were identified (by conventional recombination in female meiosis) that lacked both P-elements (white-eyed progeny). SNPs in the interval were identified that allowed for distinguishing the parent chromosome used in the screen from the chromosome that had the hedgehog P-element insertion. These SNPs allowed for identification of the regions where the crossovers had occurred. Using this type of analysis salvador was localized to a region of less than 30 kb. At this stage, the Drosophila genomic sequence became available. Two candidate genes in that region included the Drosophila p53 homologue and a transcription unit identified as CG13832. p53 sequences from each salvador mutant chromosome were amplified. No mutations were identified. The CG13832 gene was then sequenced. Each of the salvador alleles had a truncating mutation in this ORF.

Five independent cDNA clones of CG13831 were examined by restriction mapping, and two independent clones were sequenced completely. The longest clone was 2.2 kb long, which is in agreement with the approximate size of the RNA determined by Northern blotting. The predicted ORF, encoding a protein of 608 amino acids, included the entire coding region since there is a stop codon upstream and in-frame with the ATG codon.

The predicted Salvador protein has two WW domains, and its C-terminal portion includes a domain that is likely to adopt the conformation of a coiled-coil. Salvador is most similar to the human protein hWW45 (Valverde, P. (2000) Biochem. Biophys. Res. Commun. 276, 990-998) and to the protein encoded by the C. elegans ORF T10H10.3. WW domains are known to mediate protein-protein interactions with various proline containing motifs (Kato, Y., et al. (2001) J. Biol. Chem. 277, 10173-10177).

The more C-terminal WW domain of hWW45 lacks the second conserved tryptophan residue that is required for substrate binding and may not be a functional WW domain. The N-terminal WW domain contains all of the appropriate conserved residues. This putative WW domain is predicted to belong to the Group I family of WW domains that is predicted to interact with the PPXY (“PY”) motif.

Although not wishing to be bound by theory, it is believed that salvador and warts interact via a WW domain-PY motif-dependent interaction and function to promote cell cycle exit and apoptosis during development. However, warts may have salvador-independent functions as well. While salvador mutations appeared to result in a subtle increase in growth rate, the very strong overrepresentation of warts mutant tissue in third instar larval discs indicated that warts mutations must cause a much greater increase in growth rate.

The CG13832 ORF encodes a 386 amino acid protein which contains two WW domains and a putative coiled-coil domain. The original annotation of the gene in the Gadfly is incomplete and a more recently deposited EST includes the entire coding region. The coding region is from nucleotide 81 to 1907. The salvador¹ allele changes codon 289 from a Gln (CAG) to a stop (TAG). The salvador² changes codon 231 (or 232) from a Gln (CAG) to a stop (TAG) and the salvador³ allele introduces a frame-shift in codon 407 by the deletion of a G nucleotide. Thus each mutation results in the synthesis of a truncated protein that lacks both WW repeats.

hWW45 displays strong sequence similarity to salvador at the amino acid level and shows an identical organization of domains. Over the C-terminal 188 amino acids, the two proteins display 47% identity and 54% similarity at the amino acid level. The human protein contains two WW domains from amino acids 200 to 232 and 235-267. This gene has been called hWW45 by others but its function was not known. A previous description of this human gene (Valverde, P. Biochem. Biophys. Res. Commun. 276 990-998, 2000) contains only sequence information and analysis of expression in different tissues by Northern blotting but does not provide any experimental data that suggest a function.

As described herein, hWW45 was shown to be deleted or rearranged in at least two human kidney cancer cell lines. It is anticipated that this gene is either deleted or rearranged in other cells exhibiting abnormal cellular development.

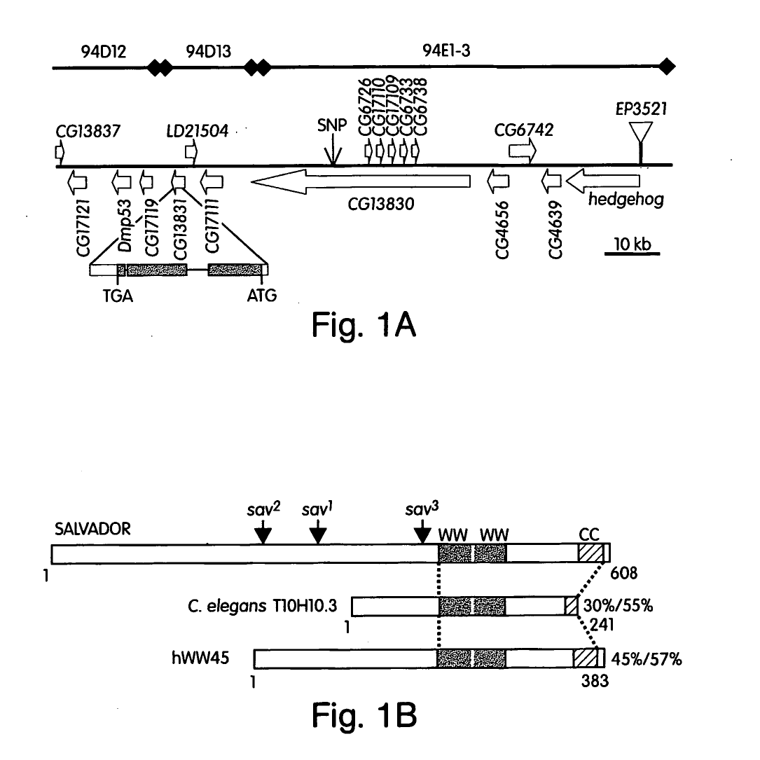

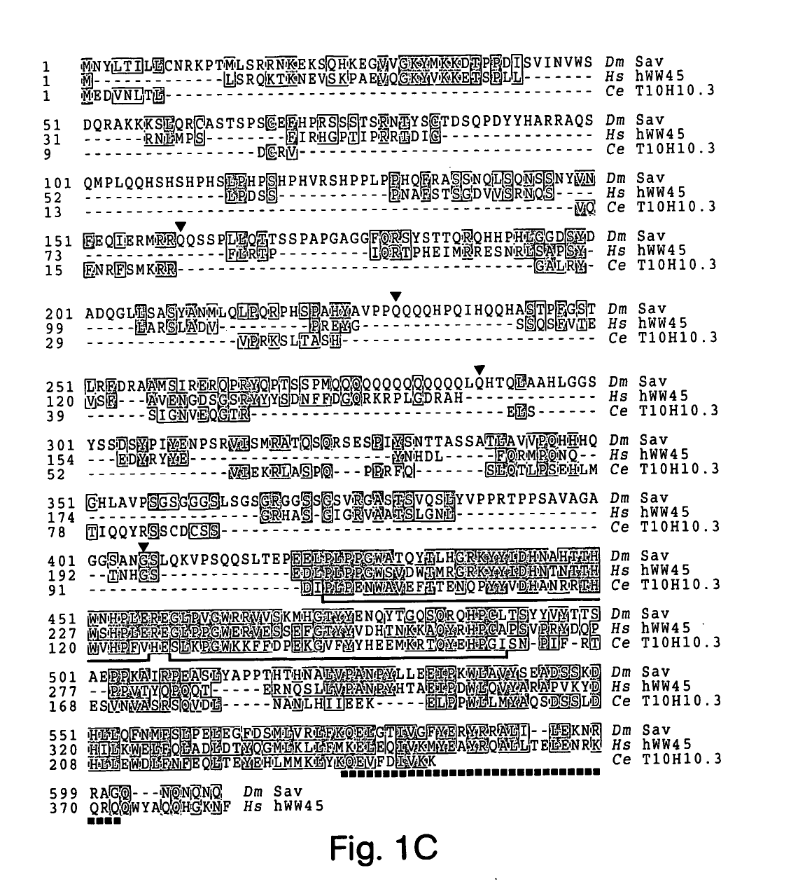

The mutations in salvador¹, salvador², and salvador⁴ result in stop codons in positions 289, 231, and 160, respectively, that truncate the protein N-terminal to the WW domains, as shown in FIG. 1. The more N-terminally salvador⁴ mutation has a more severe phenotype than salvador¹ or salvador². Surprisingly, the salvador³ mutation, which elicited the most severe phenotype, maps 3′ to those found in salvador¹ and salvador². The salvador³ mutation causes a frameshift and generated a protein consisting of 406 salvador-encoded amino acids derived from the use of an alternate open reading frame that has no sequence similarity to any protein in the database. Although not wishing to be bound by theory, it is believed that salvador¹, salvador² and salvador⁴ proteins may have some residual activity despite the absence of the WW domains and that salvador³ is a null allele. The salvador³ allele may have a more severe phenotype because the novel C-terminal sequences may have further impaired its stability or function. Alternatively, the novel C terminus of the salvador³ protein may have conferred some neomorphic properties. Any such properties, if present, were not apparent in the presence of the wild-type protein, since salvador³/+ flies displayed no overt phenotypic abnormalities. In different transheterozygous combinations, salvador³ was similar in strength to a deletion. In four independent experiments, salvador¹/salvador³ animals and salvador¹/Df(3R)EB6 animals had hatching rates of 85.5% (SD 2.5%) and 83.3% (SD 3.2%), respectively (n=40), and 90%-95% of the animals of each genotype subsequently failed to grow and died as first instar larvae. Thus, at least by this criterion, salvador³ behaved like a null mutation. The abnormalities in cell proliferation and apoptosis were analyzed using at least two different salvador alleles and only quantitative differences were observed between salvador³ and the weaker alleles.

In the eye disc, salvador was expressed in a stripe in the MF, and expression decreased in the region of the SMW. Expression increased once again posterior to the SMW. Thus, to a first approximation, salvador expression coincided with regions of temporary or permanent cell cycle arrest which supports the theory that salvador functions in promoting exit from the cell cycle.

### Example 6

### Salvador Functions Together with Warts

Clones of cells mutant for warts generate large tumor-like growths in Drosophila (Bryant, P. J., et al. (1993) Devel. Suppl, 239-249; Justice, R. W., et al. (1995) Genes Dev. 9, 534-546; Xu, T., et al. (1995) Development 121, 1053-1063). Its human ortholog LATS1 binds to the cdc2 protein kinase in a cell cycle-dependent manner and inhibits its activity (Tao, W., et al. (1999) Nat. Genet. 21, 177-181). Thus, it has been suggested that excessive Cyclin A/cdc2 may cause excessive cell proliferation by promoting both the G1/S and G2/M transitions. The interaction between warts and cdc2, however, does not explain the excessive and inappropriate growth (mass accumulation) that appears to drive the cell proliferation in clones of warts mutant cells. The defect in cell death in warts cells is also not easily accounted for by the interaction of Warts with cdc2.

One candidate for a Salvador-interacting protein is encoded by the warts (wts; also known as LATS) gene (Bryant, P. J., et al. (1993) Devel. Suppl, 239-2495; Justice, R. W., et al. (1995) Genes Dev. 9, 534-546; Xu, T., et al. (1995) Development 121, 1053-1063) that encodes a serine-threonine kinase. Clones of warts tissue generated outgrowths that resembled tumors. Nine alleles of warts were identified in our screen, and the phenotype of salvador³ was similar to that elicited by hypomorphic mutations in warts. Null alleles of warts displayed a more severe phenotype. Like salvador, warts clones in the pupal retina had additional interommatidial cells. Larval imaginal discs containing large warts clones are enlarged and convoluted (Justice, R. W., et al. (1995) Genes Dev. 9, 534-546; Xu, T., et al. (1995) Development 121, 1053-1063). Larval eye discs that contain eyFLP-induced warts clones were composed mostly of mutant tissue with small regions of wild-type tissue. Many additional BrdU-incorporating nuclei were observed in mutant clones posterior to the SMW. As observed with salvador, the stripe of cyclin E RNA expression was also broadened in these discs. Moreover, the normal cell death that occurs in the pupal retina was almost completely abolished in warts mutant clones. Thus, as for salvador, warts mutations generate additional interommatidial cells resulting from both increased cell proliferation posterior to the SMW as well as reduced apoptosis in the pupal retina. In addition, Drice activation induced by GMR-hid is markedly diminished in warts clones.

Overexpression of salvador alone using the GMR promoter (Hay, B. A., et al. (1995) Cell 83, 1253-1262) had no effect, and overexpression of warts generated subtle irregularities in ommatidial architecture. However, combined overexpression of salvador and warts resulted in a smaller eye where the ommatidial pattern is highly irregular. This effect appeared to reflect a synergistic increase in cell death in the eye discs of flies that express both transgenes as well as a minor effect on reducing cell proliferation associated with the SMW.

Thus, although not wishing to be bound by theory, it is believed that salvador and warts may function in the same pathway and bind to each other. Indeed, the Salvador protein has a Group I WW domain that is predicted to interact with the PPXY (PY) motif, five of which are found in the Warts protein. To test whether Drosophila Salvador and Warts proteins could physically interact, a GST pull-down assay was employed. The region containing the two potential WW domains of Salvador was fused to GST and incubated with cell lysates that expressed Myc-tagged Warts protein. Using this assay, Warts was found to interact specifically with the region of Salvador that contained the WW domain. Furthermore, a 15 amino acid peptide, designed to mimic one of the PY motifs of Warts, was found to inhibit the interaction between the WW domain region of Salvador and Warts. An identical peptide where the tyrosine residue that is required for interaction with type I WW domains had been replaced by an alanine did not prevent this interaction. Thus, at least under the conditions of this experiment, Salvador and Warts interact in a WW domain- and PY motif-dependent fashion. It is believed that an analogous interaction occurs in vivo.

Discs containing clones of the warts null allele, warts^{latX1} (Xu, T., et al. (1995) Development 121, 1053-1063), are much larger than discs containing salvador³ clones. If all salvador functions were warts dependent, the double mutant phenotype would not be more severe than the warts phenotype. When mutant clones were generated with eyFLP, average disc sizes were 39,669 pixels (SD 10,401) for salvador³, warts^{latsX1} double mutant discs and 31,360 pixels (SD 5260) for warts^{latsX1} discs (n=20). Thus, the double mutant discs were significantly larger than the warts^{latsX1} discs (p<0.01). Thus, while salvador and warts appear to function together in certain ways, they are believed to have functions that are independent of each other as well.

### Example 7

### The Human Ortholog of Salvador, hWW45, is Mutated in Cancer Cell Lines

Since mutations in salvador lead to excessive cell proliferation and reduced cell death, hWW45 was tested to see if it is a mutational target in cancer. hWW45 maps to the chromosomal region 14q13-14q23 (Valverde, P. (2000) Biochem. Biophys. Res. Commun. 276, 990-998), a locus that is subject to allelic loss in a variety of cancers, including renal cancers, ovarian cancers, and malignant mesothelioma. The entire coding region of hWW45 in a panel of 52 tumor-derived cell lines representing a broad range of tissue types was sequenced. One colon cancer cell line, HCT15, had a heterozygous C to A mutation at nucleotide 554, resulting in a substitution of aspartic acid for alanine at codon 185. This mutation was not present in 185 population-based controls (370 chromosomes), indicating that it is not a common polymorphism. HCT15 carries a mutation in the mismatch repair gene MSH6, which appears to enhance the frequency of point mutations in other genes. More significantly, two renal cancer cell lines, ACHN and 786-O, were found to have deletions involving hWW45. The normal allele was not present in either cell line, indicating that these cell lines are either homozygous or hemizygous for the deletion. The hWW45 transcript was undetectable by RT-PCR in both cell lines, and a Southern blot using a probe derived from the 3′ portion of the gene demonstrated that this part of the gene was absent in both cell lines. In cell line 786-O, PCR analysis of genomic DNA indicated that there is a deletion of ˜157 kb with the 5′ breakpoint between exons 2 and 3 of hWW45. The deletion in ACHN of ˜138 kb encompassed the entire gene. The common region of overlap between these two deletions is only 21 kb, containing exons 3-5 of hWW45. No other transcription units were identified within this 21 kb interval, using the GENSCAN exon prediction program. Thus, deletions that inactivate the human ortholog of salvador were identified in at least two cancer cell lines.

### Example 8

### Role of Salvador in Promoting Cell Cycle Exit

In the eye disc, salvador clones contain cells that continued to proliferate for 12-24 hr after their normal counterparts stop dividing. Studies of cycling cells showed almost no differences between wild-type and mutant populations. However, given that mutant clones contain more ommatidia than wild-type twin spots, accelerated growth must have occurred in mutant tissue anterior to the furrow. Even a relatively minor growth advantage exhibited by mutant cells at every cell cycle can eventually result in increased clone size when amplified by the approximately nine rounds of cell division that occur in the eye primordium prior to the passage of the MF. A subtle change in cell cycle parameters may not be easily detectable.

In salvador clones, elevated Cyclin E protein levels were observed in the basal nuclei posterior to the MF in the eye imaginal disc. These cells normally stop dividing when they downregulate Cyclin E protein levels. In discs containing many salvador clones, the stripe of cyclin E RNA expression is broader and more intense. Thus, the increased level of Cyclin E protein was, at least in part, a result of elevated cyclin E RNA levels. Thus, an inability to downregulate Cyclin E/cdk activity may be the result of increased levels of cyclin E RNA as occurs in salvador clones, impaired protein degradation (Moberg, K. H., et al. (2001) Nature 413, 311-316), or reduced levels of the cdk inhibitor Dacapo (de Nooij, J. C., et al. (1996) Cell 87, 1237-1247; Lane, M. E., et al. (1996) Cell 87, 1225-1235). In each case, cell cycle exit is delayed.

### Example 9

### Role of Salvador in Regulating Cell Death

Elevated DIAP1 levels were likely to underlie the absence of the developmentally regulated apoptosis in salvador clones in the pupal retina as well as the resistance to hid-induced and rpr-induced apoptosis in the larval imaginal disc. The elevated DIAP1 levels appeared to result from alterations in posttranscriptional regulation of DIAP1 expression. Recent work has shown that both Rpr and Hid can downregulate DIAP1 levels either by promoting the autoubiquitination of DIAP1 or by causing a generalized inhibition of translation that especially impacts proteins with a short half-life such as DIAP1 (Hays, R., et al. (2002) Nat. Cell Biol. 4, 425-431; Holley, C. L., et al. (2002) Nat. Cell Biol. 4, 439-444; Ryoo, H. D., et al. (2002) Nat. Cell Biol. 4, 432-438; Wilson, R., et al. (2002) Nat. Cell Biol. 4, 445-450; Wing, J. P., et al. (2002) Nat. Cell Biol. 4, 451-456; Yoo, S. J., et al. (2002) Nat. Cell Biol. 4, 416-424). Either of these mechanisms is likely to be less efficient in cells that already have elevated levels of DIAP1.

The findings described herein indicate that Salvador normally functions to downregulate the basal level of DIAP1 protein. In the absence of Salvador, higher levels of DIAP1 accumulate. This increases the level of Hid or Rpr activity that is required to overcome DIAP1-mediated inhibition of caspase activation. Consistent with this model, the more potent form of Hid, Hid-Ala5, is able to partially overcome the increased levels of DIAP1 in salvador clones and induce a low level of caspase activity.

It is believed that Salvador is capable of regulating both cell cycle exit and apoptosis by virtue of its ability to modulate the levels of two key regulators—Cyclin E and DIAP1. Loss of salvador appears to increase cyclin E levels transcriptionally and DIAP1 levels by a posttranscriptional mechansim. Since cell number is determined by both the extent of cell proliferation as well as apoptosis, Salvador could function as a key regulator of cell number by virtue of its ability to regulate both processes.

One of few pathways that can directly regulate both cell proliferation and cell death is the Ras/MAPK pathway. Ras can promote cell proliferation by promoting growth (Prober, D. A. and Egar, B. A. (2000) Cell 100, 435446), and MAP kinase can phosphorylate and inactivate Hid and also reduce Hid transcription (Bergmann, A., et al. (1998) Cell 95, 331-341; Kurada, P. and White K. (1998) Cell 95, 319-329). Although not wishing to be bound by theory, the results described herein indicate that Salvador might function in a distinct pathway. First, no change in diphospho-ERK activity was observed in salvador mutant clones. Second, cell death induced by the MAP kinase-resistant Hid-Ala5 protein (where five putative MAPK phosphorylation sites have been mutated to alanines) was also reduced by a loss of salvador function. However, it is still possible that Salvador might function downstream of the MAPK family proteins.

### Example 10

### Salvador and Warts Orthologs as Tumor Suppressors in Humans

Mice lacking the warts ortholog LATS1 display pituitary hyperplasia and develop slow-growing tumors (St. John, M. A., et al. (1999) Nat. Genet. 21, 182-186). This contrasts with the dramatic overgowth phenotype observed in warts mutants in Drosophila. It is believed that these differences may be due to the presence of other warts homologs (e.g., LATS2) in mammals that can partially compensate for LATS1 inactivation (St. John, M. A., et al. (1999) Nat. Genet. 21, 182-186).

Although not wishing to be bound by theory, it is believed that the presence of a single salvador homolog, hWW45, in humans makes it less likely that its function is redundant with that of a related gene. Mutations in this gene in three cancer cell lines have been identified and it has been shown that two of these cell lines have homozygous deletions that either disrupt or eliminate the gene. While cell lines can accumulate mutations in culture, these findings nevertheless represent a first step in implicating hWW45 in the pathogenesis of human cancer.

Athough chromosomal aberrations have been consistently identified for a number of human tumors, in most cases the relevant lesion has not been molecularly characterized. Many mammalian tumor suppressor genes must exist that have not yet been identified. The phenotype-based screen described herein, which is capable of detecting even subtle increases in growth or cell proliferation, has identified a number of genes that restrict growth or cell number. For ago and salvador, mutations in their human orrthologs in cancer cell lines have been subsequently identified. Thus, the strategy of conducting phenotype-based screens in model organisms followed by a search for mutations in cancer cell lines may be used to identify new tumor suppressor genes.

### Example 12

### Experimental Procedures

Fly Stocks

w; FRT82B males were mutagenized with ethylmethanesulfonate (EMS), then crossed either to y w eyFLP; FRT82B P[mini-w, arm-LacZ] or first to w; TM3/TM6B and then individually toy w eyFLP; FRT82B P[mini-w, armLacZ] (Tapon, N., et al. (2001) Cell 105, 345-355). Males with mostly white eyes were retained and maintained as balanced stocks. Alleles of salvador identified were salvador¹, salvador², and salvador³. GMR-hid and 2XGMR-rpr (on the second chromosome) were provided by Kristin White. GMR-hid Ala5 (second chromosome) was provided by Andreas Bergmann. FRT82B LATS^{X1} has been described (Xu, T., et al. (1995) Development 121, 1053-1063). Warts^{MGH1}, identified in our screen, is a homozygous lethal allele of moderate strength.

Mapping

Salvador mutations failed to complement the lethality of Df(3R)hh, which deleted 93F11-13 to 94D10-13. Using P element-mediated male recombination, the salvador¹ allele was placed in cis to P[lacW]C2-3-33 at 94D. The P[lacW]C2-3-33, salvador¹ chromosome was placed in trans to the P[EP]3251 (distal to salvador) chromosome in females. Meiotic recombination events were selected for between the two P elements. A SNP 57 kb proximal to P[EP]3251 was identified. Some crossovers proximal to the SNP were salvador⁺, indicating that salvador was proximal to the SNP. Of the salvador⁺ lines, 14 of 19 lines had the polymorphic variant from the P[EP]3251 chromosome, while 5 of 19 had the salvador¹ chromosome version. Since the SNP was 57 kb away from P[EP]3251, salvador is likely to be located approximately 20 kb proximal to the SNP. Genomic DNA from the salvador chromosomes for five predicted ORFs in this region was sequenced. All these ORFs were found to be wild-type except CG13831, which had a nonsense mutation in each salvador chromosome.

Microscopy, Immunohistochemistry, Flow Cytometry

For adult eye pictures, sections, and eye SEMs, genotypes were as follows: y w, eyFLP/+; FRT82B /FRT82B P[mini-w] P[armLacZ] and y w, eyFLP/+; FRT82B salvador^{1/3}/FRT82B P[mini-w] P[armLacZ]. For thorax SEMs, genotypes were y w, hsFLP/+; FRT82B/FRT82B P[πMyc] P[w y] and y w, hsFLP/+; FRT82B salvador³/FRT82B P[7πMyc] P[w y].

Imaginal disc BrdU incorporations used a 1.5 hr BrdU pulse to visualize ectopic S phases posterior to the MF. Antibodies used were anti-rabbit-Cy5 and anti-mouse Cy3 (Jackson Laboratories, West Grove, Pa.), a rabbit polyclonal anti-phosH3 antibody (Upstate Laboratories, lake Placid, N.Y.), anti-β-galactosidase rabbit polyclonal (Cappel, Aurora, Ohio), a mouse monoclonal anti-β-galactosidase (Promega, Madison, Wis.), and a mouse monoclonal is anti-DIAP1 antibody and a rabbit anti-activated Drice antibody (both provided by Bruce Hay) (Yoo, S. J., et al. (2002) Nat. Cell Biol. 4, 416424). FACS analysis was performed as described previously (Neufeld, T. P., et al. (1998) Cell 93, 1183-1193; Tapon, N., et al. (2001) Cell 105, 345-355).

For immunofluorescence and TUNEL stainings, discs were dissected from the following genotypes: (1) y w, eyFLP/+; FRT82B salvador^{1/2/3}/FRT82B P[mini-w] P[armLacZ], (2) y w eyFLP/+; FRT82B Warts^{MGH1}/FRT82B P[mini-w] P[armLacZ], and (3) y w eyFLP/+; FRT82B LATSX1/FRT82B P[mini-w] P[armLacZ]. For TUNEL, DIAP1, or Drice stainings and adult eye pictures in a GMR-hid transgenic background, genotypes were y w, eyFLP/+; GMR hid/+; FRT82B/FRT82B P[mini-w] P[armLacZ] and y w, eyFLP/+; GMR-hid/+; FRT82B salvador³/FRT82B P[mini-w] P[armLacZ]. For DIAP1 or Drice in a GMR-rpr or GMR-hid-Ala5 transgenic background, genotypes were y w, eyFLP/+; GMR-hid/+; FRT82B /FR182B P[mini-w] P]armLacZ] and y w, eyFLP/+; GMR hidAla5 (or 2XGMRrpr)/+; FRT82B salvador³/FRT82B P[mini-w] P[UbiGFP]. TUNEL stainings were performed as previously described (Kurada, P. and White K. (1998) Cell 95, 319-329). TUNEL positive nuclei were detected with a Rhodamine-conjugated anti-DIG antibody (Boerhinger, Indianapolis, Ind.). For FACS analysis, the genotype was y w, eyFLP/+; FRT82B salvador³/FRT82B P[mini-w] P[UbiGFP].

Loss-of-function wing clone counts were performed as previously described (Tapon, N., et al. (2001) Cell 105, 345-355). Clones were induced at 48 hr after egg deposition (AED). Discs were dissected for analysis at 120 hr AED. The genotype was y w, hsFLP/+; FRT82B salvador³/FRY82B P[mini-w] P[UbiGFP].

Molecular Biology

The coding region of a salvador cDNA clone was PCR amplified using oligonucleotide primers with EcoRI and BgIII sites and cloned into pGMR. A 4.1 kb EcoRI/DraI fragment of the warts cDNA (provided by Peter Bryant) was cloned into pGMR. GMR-salvador and GMR-warts were third chromosome integrations.

Characterization of Human hWW45

The entire coding region of hWW45 was amplified by RT-PCR in two overlapping fragments. Uncloned PCR products were sequenced directly. The cancer cell lines analyzed is and the DNA used for control populations were performed as described previously (Moberg, K. H., et al. (2001) Nature 413, 311-316).

Primers derived from intronic sequences were used to amplify individual exons of hWW45 from genomic DNA of the ACHN and 780-O cell lines to assess the extent of genomic deletions in these two cases. For the regions flanking hWW45, primers based on nonrepetitive sequences from BACs containing hWW45 were used.

Protein Binding Studies

The Drosophila warts gene was Myc tagged and cloned into the pCDNA3 mammalian expression vector. The Salvador WW domain-GST construct was generated by cloning sequences encoding residues 419495 into the BamHI/EcoRI sites of pGEX-2TK. 293T cells were transfected using Fugene and harvested 36 hr later in lysis buffer (50 mM Tris-HCl [pH 7.5], 150 mM NaCl, 10 mM EDTA, 10% glycerol, 1% Triton X-100, 20 mg/ml leupeptin, 10 mg/ml aprotitin, 1 mM PMSF, 0.5 mM DTT, 0.5 mM NaF). Cell lysates were incubated with 500 ng to 1 mg of GST-fusion protein coupled to glutathione-sepharose, in the presence or absence of peptides (1 mM) for 2 hr at 4° C. Peptides were PY-GRQMLPPPPYQSNNN and PAGRQMLPPPPAQSNNN (SEQ ID NO: 25 and SEQ ID NO: 26, respectively). Beads were then washed, treated with protein sample buffer, and subjected to SDS-PAGE. Warts protein was detected by immunoblotting with anti-Myc tag 9E10 mAb.

Standard co-immunoprecipitation protocol using 293T cells were used to identify agents that bind to Salvador molecules, e.g., warts/LATS. Cells were maintained in Dulbecco's Modified Eagles Medium (DMEM) with 10% Fetal Calf Serum (FCS). Cells were transfected using Fugene (Roche) according to the manufacturer's instructions with the following plasmids: pCDNA3-HA-Salvador (full-length) and pCDNA3-Myc-warts/LATS (Full-length). Cells were harvested after 48 hours into 400 μl of lysis buffer (40 mM Tris-HCl pH7.5, 50 mM NaCl, 50 mM NaF, 100 μM NaVO₄, 0.2% NP-40, 10 μg each aprotinin and leupeptin). Insoluble material was removed by centrifugation at 4° C. for 10 minutes at 10,000 g. 50 μl of lysate was used per assay. Myc-tagged warts/LATS was precipitated with 9E 10 antibodies and 12CA5 was used for HA-tagged Salvador. 20 μl of protein A-sepharose were used per assay. The immunoprecipitates were washed 3 times for 15 minutes with lysis buffer at 4° C., then subjected to SDS-PAGE (12% gel), transferred to a nitrocellulose filter and analysed by Western blotting using the same antibodies used for the immunoprecipitation.

Methods for Screening Human Cells for a Salvador Molecule

To prepare for the Southern Blot Analysis of human tumor cell lines, the following protocol was followed: 10 μg of genomic DNA from samples of human tumor cell lines and control tissues were cut and run on a 0.8-1% agarose gel at 100 V for 4 to 6 hours. Afterwards, a picture was taken with a ruler next to the gel. The gel was denatured in 1 liter of denaturing solution (88 g/L NaCl and 20 g/L NaOH) for 35 to 40 minutes at which point the dye changed color. The gel was rinsed with water and neutralized with 1 liter of neutralizing solution (60.55 g/L Tris base and 87.6 g/L NaCl, pH 7.2, RT) twice for 20 minutes. The gel was transferred onto a Hybond N+ membrane (Amarsham Pharmacia, Piscataway, N.J.) with 10×SSC solution (87.65 g/L NaCl, 44.1 g/L sodium citrate, pH 7.2, RT). The well positions were marked with a pencil after transfer. The membrane was placed, DNA side up, on a piece of Whatman paper and allowed to absorb the superficial liquid for 20 seconds. The DNA was crosslinked to a damp membrane using 120,000 μJ using standard autocrosslinking procedures with a Stratagene UV oven. The resultant membrane, ready for prehybridization, is optionally stored dried between two pieces of Whatman paper for several months in the dark.

The block was prehybridized in 15-20 ml (0.2 ml/cm²) of prehybridization buffer at 68° C. for at least 2 hours in a sealed bag. Prehybridization buffer contains 6×SSPE and 0.05× blotto. 20×SSPE contains 175.3 g/L NaCl, 27.6 g/L NaH₂PO₄, and 7.4 g/L EDTA, pH 7.4, RT 1× blotto. Blotto contains 5 g of non-fat dried milk powder in 100 ml of water and 0.02% NaN₃, stored at 4° C. 20 ml of prehybridization buffer contains 6 ml of SSPE 20×, 1 ml of Blotto 1×, and 13 ml of water.

Hybridization proceeded overnight at 68° C. in a sealed bag with 15 ml of hybridizaton solution. Hybridization solution includes 6×SSPE, 0.5% SDS, 100 μg/ml salmon sperm DNA, and no more than 2×10⁶ cpm/ml of hybridization solution of P³² labeled probe. 15 ml of hybridization solution contained 4.5 ml SSPE 20×, 0.375 ml of 20% SDS, 150 ml of salmon sperm DNA (10 mg/ml denatured by boiling), 9.975 ml of water, and the probe, which had been denatured by boiling.

The blot was rinsed for 5 minutes with 50 ml 2×SSPE and 12.5 ml of 20% SCF. The blot was then washed for 15 minutes with 2.5 ml of 20% SDS. The blot was further washed for 1 hour with 2.5 ml of 20×SSC and 12.5% SDS. The membrane was wrapped in plastic wrap and exposed for at least 16 hours at −80° C. with an enhancing screen.

The probe (SEQ ID NO:5) was made on oligolabeling kit (Amersham, Pharmacia). The DNA to be labeled was boiled and 50 ng were added to an eppendorf tube along with 10 μl of reagent mix, 5 μl of a P³² dCTP (3000 mCi/mmol), and 1 ml Klenow enzyme. Water was added to bring the volume to 49 μll. After mixing the reagents, the eppendorf tube was incubated at 37° C. for 1 hour. After incubation, the unincorporated nucleotides were discarded by passing the reaction through a Sephadex G50 column. Since 5 μl of a P³² dCTP (3000 mCi/mmol) is equivalent to 100 million cpm, the signals from the column to that of the reaction were compared to evaluate the amount of incorporated a P³² dCTP. No more than 2 million cpm/ml were placed in the hybridization solution.

All references disclosed herein are incorporated by reference in their entirety.

1. A method for treating a subject with a disorder characterized by aberrant expression of a salvador molecule, comprising:
administering to the subject an effective amount of a Salvador nucleic acid molecule to treat the disorder.

2. The method of claim 1, wherein the disorder is a cancer.

3. The method of claim 1, wherein the disorder is an autoimmune disorder.

4. The method of claim 1, wherein the disorder is a birth defect.

5. A method for treating a subject with a disorder characterized by aberrant expression of a salvador molecule, comprising:
administering to the subject an effective amount of a Salvador protein to treat the disorder.

6-8. (canceled)

9. A method for diagnosing a disorder characterized by aberrant expression of a Salvador molecule, comprising:
detecting in a first biological sample obtained from a subject, expression of a salvador molecule or a Mutant Salvador molecule;
wherein decreased expression of the salvador molecule or detectable expression of the Mutant salvador molecule compared to a control sample indicates that the subject has the disorder characterized by aberrant expression of the salvador molecule.

10-34. (canceled)

35. An isolated nucleic acid molecule selected from the group consisting of:
(a) nucleic acid molecules which hybridize under stringent conditions to a nucleic acid molecule having a nucleotide sequence set forth as SEQ ID NO:3, and which code for a Salvador protein,
(b) deletions, additions and substitutions of the nucleic acid molecules of (a), which code for a Salvador protein,
(c) nucleic acid molecules that differ from the nucleic acid molecules of (a) and (b) in codon sequence due to the degeneracy of the genetic code, and
(d) complements of (a), (b) or (c).

36. (canceled)

37. An isolated nucleic acid molecule selected from the group consisting of:
(a) a unique fragment of the nucleotide sequence set forth as SEQ ID NO:1 or set forth as SEQ ID NO:3 between 12 and 2000 nucleotides in length and
(b) complements of (a),
wherein the unique fragments exclude nucleic acids having nucleotide sequences that are contained within SEQ ID NO:1 or SEQ ID NO:3 and that are known as of the priority date of this application.

38-40. (canceled)

41. An isolated nucleic acid molecule selected from the group consisting of:
(a) nucleic acid molecules which hybridize under stringent conditions to a nucleic acid molecule having a nucleotide sequence set forth as SEQ ID NO:6, and which codes for a Salvador protein,
(b) deletions, additions and substitutions of the nucleic acid molecules of (a), which code for a Salvador protein,
(c) nucleic acid molecules that differ from the nucleic acid molecules of (a) and (b) in codon sequence due to the degeneracy of the genetic code, and
(d) complements of (a), (b) or (c).

42. An isolated nucleic acid molecule selected from the group consisting of:
(a) a unique fragment of the nucleotide sequence set forth as SEQ ID NO:6 between 12 and 2000 nucleotides in length, and
(b) complements of (a),
wherein the unique fragments exclude nucleic acids having nucleotide sequences that are contained within SEQ ID NO:6 and that are known as of the priority date of this application.

43-44. (canceled)

45. An isolated nucleic acid molecule selected from the group consisting of:
(a) nucleic acid molecules which hybridize under stringent conditions to a nucleic acid molecule having a nucleotide sequence set forth as SEQ ID NO:1 or set forth as SEQ ID NO:3, and which code for a Mutant Salvador protein,
(b) deletions, additions and substitutions of the nucleic acid molecules of (a), which code for a Mutant Salvador protein,
(c) nucleic acid molecules that differ from the nucleic acid molecules of (a) and (b) in codon sequence due to the degeneracy of the genetic code, and
(d) complements of (a), (b) or (c).

46. (canceled)

47. An expression vector comprising the isolated nucleic acid molecule of claim 35 operably linked to a promoter.

48. An expression vector comprising isolated nucleic acid molecules selected from the group consisting of:
(a) nucleic acid molecules which hybridize under stringent conditions to a nucleic acid molecule having a nucleotide sequence set forth as SEQ ID NO:1, and which code for a Salvador protein,
(b) deletions, additions and substitutions of the nucleic acid molecules of (a), which code for a Salvador protein,
(c) nucleic acid molecule that differ from the nucleic acid molecules of (a) and (b) in codon sequence due to the degeneracy of the genetic code, and
(d) complements of (a), (b) or (c),
operably linked to a promoter.

49. A host cell transformed or transfected with the expression vector of claim 48.

50. A transgenic non-human animal comprising the expression vector of claim 48.

51-54. (canceled)

55. An isolated polypeptide encoded by the isolated nucleic acid molecule of claim 35.

56-85. (canceled)

86. A method to induce apoptosis in a cell, comprising:
administering to the cell an effective amount of a salvador molecule to induce apoptosis in the cell.

87-90. (canceled)

91. A method to inhibit cellular development of a cell comprising:
administering to the cell an effective amount of a salvador molecule to inhibit cellular development of the cell.

92-95. (canceled)

96. A method for identifying a salvador molecule comprising:
(a) introducing a putative salvador molecule or a putative Mutant salvador molecule into a cell; and
(b) detecting a salvador functional activity selected from the group consisting of binding to a warts/LATS molecule, modulating cell maturation/differentiation, modulating cell growth, modulating cell proliferation, and modulating cell death.

97-100. (canceled)

101. A method for identifying salvador modulating agents that modulate a salvador molecule-cognate interaction, comprising:
(a) contacting a salvador molecule with a cognate under conditions to allow the salvador molecule to bind to the cognate, in the presence of a putative modulating agent; wherein the cognate is a warts/LATS molecule; and
(b) detecting salvador molecule binding to the cognate;
wherein a change in salvador molecule binding to the cognate in the presence of the putative modulating agent compared to salvador molecule binding to the cognate in the absence of the cognate indicates that the agent is a salvador modulating agent.

102. (canceled)


SEQUENCE LISTING (26 sequences)

SEQ ID NO: 1 - DNA, 2902 bp, Homo sapiens
tgaccagcgactgagcggcggccggcgcgcttagcgccctgaacatgcggcagtccctgcgggcgaccccgggctccggacaggcggcggcggaggcggcggctcgggagggaaggaggcggcggcgccggcggaggtggcggcggagacggccggcgcccggcgcggagccctagggaggcagttcagcgcggcctcgggcctcgtcgagaaggatgctgtcccgaaagaaaaccaaaaacgaagtgtccaagccggccgaggtgcaggggaagtacgtgaagaaggagacgtcgcctctgcttcggaatcttatgccttcattcatccggcatggtccaacaattccaagacgaactgatatctgtcttccagattcaagccctaatgccttttcaacttctggagatgtagtttcaagaaaccagagtttccttagaactccaattcaaagaacacctcatgaaataatgagaagagaaagcaacagattatctgcaccttcttatcttgccagaagtctagcagatgtccctagagagtatggttcttctcagtcatttgtaacggaagttagttttgctgttgaaaatggagactctggttcccgatattattattcagacaatttttttgatggtcagagaaagcggccacttggagatcgtgcacatgaagactacagatattatgaatacaaccatgatctcttccaaagaatgccacagaatcaggggaggcatgcttcaggtattgggagagttgctgctacatctttaggaaatttgactaaccatggttctgaagatttaccccttcctcctggctggtctgtggactggacaatgagagggagaaaatattatatagatcataacacaaatacaactcactggagccatcctcttgagcgagaaggacttcctcctggatgggaacgagttgagtcatccgaatttggaacctattatgtagatcacacaaataagaaggcccaatacaggcatccctgtgctcctagtgtacctcggtatgatcaaccacctcctgtcacataccagccacagcaaactgaaagaaatcagtcccttctggtacctgcaaatccatatcatactgcagaaattcctgactggcttcaggtttacgcacgagcccctgtgaaatatgaccacattctgaagtgggaactcttccagctggctgacctggatacataccagggaatgctaaagttgctcttcatgaaagaattggagcagattgttaaaatgtatgaagcatacagacaagcccttcttacagagttggaaaaccgaaagcagagacagcagtggtatgcccaacaacatggaaaaaatttttgagctgattttttaaaaatttaagttttgtaagagctttaaaatattttcacagataaaaaattgcaaacaagtactctggttaataaatgctgcttcctttgtggaaattataaaattctaactttacatgtattttgttattagaaattttcttttattgaatgagaaaaattagtctatcattttaagagccaatatggcaaacactttcaaatactgtatattaggaaactgttttggtattcttgatggaaaaaaatgcagcggaaatgtcattatgaacagatgttaaataggaaattattacttgttaacttcttacagcagtagtaccttctttaaaaaaaaaaaagaatctgcggtatttttttaaaaaaaaagtttactgctgtagtgtgaaatattgtctggaaagggatggtttaaatattaccatgatgtagttgaaatataaaataggatttggaaccttattgtgataaatatttatatatgatgtatgtttgtctcccctctccaaagtaaggaacccagctgggcgtggtggctcactcctgtaatcccagcattttgggaggccgaggtgggtggaccgcttgagtccacgagtttaagaccagcctgggcaacatggtgaagccctacaaaaaatacaaaaaaattagccgggtatgatggtgtgtgcctctagtcccagctacttgggaggctgaggtcagaggatggcttgagcccaagaggcagaggttgcagggtcaagatcgcaccacttgcactgcagcctgagagagagagagccagaccttgtctcaaaaaataaaaaataaaaaaataaaaacaaaataaggaatccttttaaagcatgttgagaaagtattttttggtatgaatgggtgacgggccttttaatctttcaggaatacatataagcaggaatacttagcctgtattgggaaggtgcgtgaaactcaaagttgctaacatcatcttccttattttcagccagtgctgttactgcatttagcatggctctgacgaggcagtggagagttcctgacagcatagcatactgccttctgacagactgggttaagctaccaaaaatgacaaccagggattcaaaaaaaatttttggtcattaaatcgaatgtatattattactttttaattttctaagtatttatcacaggactttaacaccatatatttccctcccagtttttctccccttttggtttaatagttaaaacattcttcataattagtgctgtacatttgattgcaaacaaattggtgatacttctaaactataactataaatctctagaattctgtgaatttcaacattatttgaagtagttcttcagttatatttacatgtgttgtaaatatgtaaatatacttttagcttaggaagtcttccagaatacaaactactcccagttagatatcagtgagtttgaataactgaagaaatgttgacaatgttaagtgtttgattttatataacaagcttattaaatatatttttgtgg

SEQ ID NO: 2 - protein, 383 aa, Homo sapiens
MLSRKKTKNEVSKPAEVQGKYVKKETSPLLRNLMPSFIRHGPTIPRRTDICLPDSSPNAFSTSGDVVSRNQSFLRTPIQRTPHEIMRRESNRLSAPSYLARSLADVPREYGSSQSFVTEVSFAVENGDSGSRYYYSDNFFDGQRKRPLGDRAHEDYRYYEYNHDLFQRMPQNQGRHASGIGRVAATSLGNLTNHGSEDLPLPPGWSVDWTMRGRKYYIDHNTNTTHWSHPLEREGLPPGWERVESSEFGTYYVDHTNKKAQYRHPCAPSVPRYDQPPPVTYQPQQTERNQSLLVPANPYHTAEIPDWLQVYARAPVKYDHILKWELFQLADLDTYQGMLKLLFMKELEQIVKMYEAYRQALLTELENRKQRQQWYAQQHGKNF

SEQ ID NO: 3 - DNA, 2212 bp, Drosophila melanogaster
cgatcgtacagcataaatttagctcgaaaatggactaaacaaagacagcgatccggaatccgagcggaaacatagtctgcatgaactatctaacgatcctgctgtgcaaccgaaaaccgacgatgctctcgcgccggaacaaggagaagtcccagcacaaggagggcgtggtggggaagtacatgaagaaggacaccccaccggatatttcggtgatcaatgtgtggagcgatcagcgggccaagaagaaatcgctgcagcgctgtgcgagcacctcgcccagctgcgagttccatccgcgcagctcgagcaccagtcggaacacctactcctgcacggactcgcagccggactactaccatgctcgacgagcacagagccagatgcccctgcagcagcactcccactcgcatcctcactctctgccccacccctcccatccgcatgtgcgtagtcatcctcccctgccgccccaccagttccgcgccagcagcaatcagttgagtcagaacagcagcaactacgttaatttcgagcagatcgagcggatgcgccgtcagcagtcgtcgccactgctgcagaccacatcatcgccggcgccgggagccggaggattccagcgcagctactccaccacccagcggcagcatcatccccatatgggtggtgacagctacgatgcagatcagggcctgctaagcgcctcctatgccaacatgttgcaactgccccagcggccacactcgcccgctcactacgccgtcccgccgcagcagcagcagcatccacagattcatcaacagcacgcctcgacgccgtttggctccacgctgcggttcgatcgagctgccatgtccatcagggagcgacagcccaggtatcagccaactagctctccgatgcagcagcaacaacaacaacaacaacagcagcagcagcagctgcagcacacacaactggcagctcacctgggcggcagctactccagcgattcgtacccgatctacgagaatccgtcccgcgtcatctcgatgcgcgccacgcagtcgcagcgatcggagtcgcccatctacagcaatacgacggcctcgtcggccacgctggccgtggttccgcagcatcatcatcagggtcacctggcggtgccatctggaagcgggggaggatccctgagcggcagcggtcgtggtggcagttctggcagtgttcgcggcgcctctacctcagtgcaatcactgtacgtcccaccgcgaactccgcccagtgcggttgccggagcgggaggcagtgccaatgggtcgctgcagaaggtaccatcacagcaatcgctcacggagcccgaggagctgcctctgccgcccggctgggccactcagtacacgctacacggtcggaaatactatattgatcacaatgcgcataccacgcactggaatcatccgttggagcgcgaaggtctgccggtgggctggcggcgggtggtgtccaagatgcatggcacctactatgagaaccagtataccgggcagagccaacgtcagcatccatgcttgacctcctactatgtctacacgacgtctgcggagccaccgaaagcgattcgaccagaggcgtcgctctatgccccacccacgcacactcacaatgcactggtgccggccaatccctatctgctcgaggagatccccaagtggttggccgtctactcggaggcggactcgtccaaggaccacctgctgcagttcaacatgtttagcctgccggagctggagggcttcgacagcatgctggtgcggctcttcaagcaggaactgggcaccatcgtgggcttctacgagcgctaccgtcgcgctttgatactcgagaagaatcgacgcgccggccagaaccagaaccaaaaccagtgacccggtgaccaggtgacgactgactcagaccacatactcgccagcagctatatgcacatcatagtgctcctgtaatcgacctttaacttatttaaccatcgactcatcgcgaaatcagtgccttatacgaaaccagacgagatggtagccaagcagatccatgacagttcgaatgccttgatgaaacgtagaattgtgctacgttctatataaccttaatgtgatttgagcttggcgtttgtttgtaatgtgagcaaagaaaattaaactggtttactgatcatcttaaaaaaaaaaaaaaaaaa

SEQ ID NO: 4 - protein, 608 aa, Drosophila melanogaster
MNYLTILLCNRKPTMLSRRNKEKSQHKEGVVGKYMKKDTPPDISVINVWSDQRAKKKSLQRCASTSPSCEFHPRSSSTSRNTYSCTDSQPDYYHARRAQSQMPLQQHSHSHPHSLPHPSHPHVRSHPPLPPHQFRASSNQLSQNSSNYVNFEQIERMRRQQSSPLLQTTSSPAPGAGGFQRSYSTTQRQHHPHMGGDSYDADQGLLSASYANMLQLPQRPHSPAHYAVPPQQQQHPQIHQQHASTPFGSTLRFDRAAMSIRERQPRYQPTSSPMQQQQQQQQQQQQQLQHTQLAAHLGGSYSSDSYPIYENPSRVISMRATQSQRSESPIYSNTTASSATLAVVPQHHHQGHLAVPSGSGGGSLSGSGRGGSSGSVRGASTSVQSLYVPPRTPPSAVAGAGGSANGSLQKVPSQQSLTEPEELPLPPGWATQYTLHGRKYYIDHNAHTTHWNHPLEREGLPVGWRRVVSKMHGTYYENQYTGQSQRQHPCLTSYYVYTTSAEPPKAIRPEASLYAPPTHTHNALVPANPYLLEEIPKWLAVYSEADSSKDHLLQFNMFSLPELEGFDSMLVRLFKQELGTIVGFYERYRRALILEKNRRAGQNQNQNQ

SEQ ID NO: 5 - DNA, 1342 bp, Homo sapiens
aaatttgactaaccatggttctgaagatttaccccttcctcctggctggtctgtggactggacaatgagagggagaaaatattatatagatcataacacaaatacaactcactggagccatcctcttgagcgagaaggacttcctcctggatgggaacgagttgagtcatccgaatttggaacctattatgtagatcacacaaataagaaggcccaatacaggcatccctgtgctcctaggtacagtatcaactttattttcaactttttgtttcgggattgtctaatgttaggttttgaactgatattttcatctaagagaatagcatcctaaagcttgagatttgaaagccttatatttcaaaatttacatttttctctggacatacatttagtaataaatgactttaataagttaaaaaaatacttttctctttaaaaaaacctcaaaaacaagatcattgttcttatgatcagattctgctgtagtaatgccagaatgtttttgttataggtgtccagaaaattaaataattaaagcttctgccttcttacacttaaatttttacaaataataacagacctaccccctaagtcagaatgtacaattgaattataacattttgctgtttattctatttcagacatgattgtatgttctgtttcttgctattactagacaagaaagtgaaaattgggactgaacaaaacatcccatttctgtttaataaatatagccagtaattctttgtgctgttttgattgatactgggctgggtagatctgctttgttgccagacttttagccctgtgctgcctgatagccagttgtcacgtgtggctgttgagcccttgaatgtgattaatataaattgagatgtgctgtaagtgtaaatacacactggatttcaaagtacataaaagctagtattagtttagtaatgaagttgcaaatatttcaaattaaccacatttgagcttcaaaagctgagccacaagattaaaaatgagtaaaatatcatctgcattttgataaatgttgcttagtgtcatctggcataatacagttttttggttatagtggacatttgtattttcttagaaccattaaaatagttatgaaaatgtgttttaggattatcaactggaaaactaagctttatcattattgtgttgttttaattattttatgtatttggatttattaattcaactttgaaaatctgttttttcccatgattccatgtagtggcacccaatcagtgtttattcaactactgaggttgttgtcctttgaaagaatcaaacctgtggtgtggtcatcaggtggcaatgattgttg

SEQ ID NO: 6 - DNA, 38045 bp, Homo sapiens
  misc_feature (11476)..(11476): n = a or g or c or t
ttagcattattgaaggcaatgtctggagaattacagctccgtagaactacagcaaatctgaattccttaataaaaattctcagaagtcaaggaatttctagaaaacaaacatgaattcaaagcttttagatttaaggattcaaattcaaagttcacatatttgttgtgcattgctatagctaatgctgcctgcagattgttgattctggaatgtaccctccttgataaaaaggcaaattagttgtcttaaatagagcacctaattaaggctttctctctgctataatccacagaaattaaatagcattgtaaaagggagtactctctaaggaacacagacggctttcaaaacaactgttagaaacagtctggactcacattagtgggattctgaatgttccgtagcagtaaagagccccaaacggaaattgaggctggcctggtggctcacgcctacaattgcagcactttgggaggccgaggcgggcagatcacttgaggccaggagttcgggaccagcctggccaacatgatgaaatccagtctctactaaaaatacaaaaaatagccaggtgtggtggcgggcgcttgtaatcctagctactcaggaggccaaggtaggaggatcgcttgaacctgagaggcagcagttgcagtgagctgagatagtgctactgcactcctgcctgagggatagagcaagactctgcctcaggggggaaaaaaagggaattgaaaactccagttctgatgctattctgtcattaaccaggaatgtaatctgagacaaaacacttaatttttctcgttcctttttgatgttgatgaggtgagcctaggcaatctcagaattctttttcaactataaaattctaggattctttcctccgcacatgtgaaaggtgtccccgtacttcaggctctgggaaaactccaggtgtgaagagttgcctttattaacaagatgctaatatccttttgtccttctctgaggtaagccttaggcagaaacctggcacttctttggctcccccttcctccttcagctcccgccttcataccctgatgtgtaacaaaaccccattgtgctctcacttaagtttcatcactctttcttctccctgcacactatcaacgctttaggactcgtccttatttctgaaccacaggaacaagtaactgtttttctaatgtttctctcagagtacactctgcttctagtgagttccaattctgtttattaagattttcatacccatggcagtgattgatactcccacactccatattcaatgtcagcaaatttggttggctttctttaaaatatgtctaaaatatgtccacttacctctccacctattattgcctgggtccaagccagcaataacttctgactgatctcccttttgcccttagctccattgtctactctccacacagcatccaaagcagtcctatgaaaacattaactcagatcatgtcactcctctgctcaaagtaacattccttacaatggcctacaaagccctaggtgccgtggctattgttactctctaactgcattccccattcgtctccccgtcttttccagtacataaaccctcttactgttcctccaatctgccaggaacatggccacttgcagcgactttgcacttgctgttctttctgcctggaatattgttcctccagttagctgcatggccaatttcctcactctgtcacttaactccaatgtcacctcctcatgagccttctctggtccctctattaccctctcaaaaattgtgagtcctctcttattttcacttcatcacttatcactatctaacatagtatgttttgcttattttttgtcatttttttctctttcccatgacagtagggatttttgtctgttttgcttattgctctatcacaaacgactagcgcagtgggtaatgagtatttcctggttaatgaatgaattgtgggagatctgcaccaagggaacatatggagacagacactagatcaaatgtctcctttgtttgctcatggtaaaacatttgaccatcttcttaatcttacgtggtaaaaaattccatgtcttttgttaattgtgatatatgtcttagatctcttgtgagatgttttagatctcagggaaagcattcctcattattaaagcattaatttggtcactaggggatgaatccaatcttattttccaaatgttatcctgtacatttggcagtcttgctaagttatatagaaggaagtagagtctattattttagatagaaaattcccggtcccgactcgcagtggtcaaagtcttcaaaattctaagatcataacacataacgtcaggaggaaatatgtggctattaatcatactaatgatagtgatgatgcaataaaataattgtaatatgctaacaccaattgtatcttaaaaagaggtactggaaattccatcttgcattcttccgttttttcctgttatagaaaaacgcactccctgatactcagtagaggatcaacttatttctagttaatcagcatgaacgtttccagcagaatagatgctttggtgtagcacttgcctgggagtcatggttagcattccgcattttttagagagctgaattcagcagcctaggaaacattaggaagaacaccttcaaacagggaacaacgtcttggggggaagagagtcactgcctccgctagggatcccgagggggtacaaaagcaacaacaacaaaaactcgtggagagcaggctccacgacatgcgcatacgacgaactcccgcactcagcggggcaggtttcgtccttcatcttctcagtcggcaggacctgcttctctctgaggatgagtgaggacagccgcagctcggcggggacggcccgttagcctagagcgccggcacccgagtcccgccgccggagccgcgcctggagccgcggtgggggaggggcagcggcggcccgcgctgcgtcgagcttcccagaattcccagcggccgcggggaaagtttacgggatgtaggcggcggcggcggtagtggtagcggttattcggcggcccgcggcggaccatggccctggcccggcgtcgctgggctttcctcacggcgtccccgagcagcgtcgcagagcgggccgacttccgggaaggaactgaccagcgactgagcggcggccggcgcgcttagcgccctgaacatgcggcagtccctgcgggcgaccccgggctccggacaggcggcggcggaggcggcggctcgggagggaaggaggcggcggcgccggcggaggtggcggcggagacggccggcgcccggcgcggagccctagggaggcagttcagcgcggcctcgggcctcgtcgagaaggatgctgtcccgaaagaaacccaaaaacgaagtgtccaagccggccgaggtgcaggggaagtacgtgaagaaggagacgtcgcctctgcttcggagtgagtgtcaggcggggcgtgcccacacctggcccaggcggccaggtgctccggggaccctggcgggggcgccggcgggtctccagggcttctccttcgtccttggtcgcgtgaaatactggttttgagattttcttgtgtctgaagtggtattcgttgattgaaagcccataagagtaagaaacttgggttagtcccccccgcgcctaacccccccaagagcaccccctaaaaataacagacttcctcgccccaccaggcagagttgttggttttgattactggaagagagagtaggaaacatttaaattgcttatcacctaatgctataggagtaaaagtaattagacagggacactttttactcttttagatgcgtcgagatgtgcgtcatgtttgcaaacattgcgggtgtctctcgactgtgctgcaagttgtgcgaactctggggcagaattccttagtcctgctgccggatgcagacttgagaaggtgaaaagcataccgtcgttcttgtagccgccacccaaagcctcatcactcttacacagcagagcagagagccaaaagcctttgtatttggctatattcgtgagattccaagaaggcaactcaaaataatttctatcgtattagagaacccccccctcgaaaagttttttttttaatagggtagatcccactcccaatccccacctccggtcccccgctgcttttcttcttaatgtaagtatttgatttttgagttgaatctcttgaatttttaattcttgtgtgttcaggacatttgtgaggcatggagctggtattttggtgctcttactgcgccctgggccctttgcttaagtttccttcttggaagctggtagtaaccttgtgacttaagtattacctcatattgtaattgggaaagttaggtttctagtatttgcccaagatcacacagatgggagagcatgtttttccatcataaccacattgtcagccttccagatttatggtggtacttgcaatactttcaacaaaatatatgtcagttgagtacatcagtaactcgaaacttgatttttaagttttttttttttttttttaacaacttgattttcagtcacacatgcagttaggtttctggctgtttttaagtgatagcttgctgtttgctgcaaataaagtttatcagacattacttgaaatcgtgaattatgtttgtcacactttttattatactttaagttctggggtacatgtgtagaacgtgcagttttgttaatgtttgtcacagttaagcaaataacttttgatcaactttgttttgttgtcgttacatcaagtgttgagccttatttattcagatatttgtttctgtccagtgttagtttctgaagcttattaatatgagtgtctccaatattgtttacttgatgaataataagagggtctgcatattcaggtttttgtcttgttttaaaggtatttgctttctaactgggaatgtcttaagctgttttcaagtttcgtgtttaaaagtactgtatcctaaaagctgttgttctattatctactattttgaattataattacatagtttcaggcttggttttttgagccacttctacatttgtgtgcttctttgaaatgagagctttttattttcaattttcatacataaatttttggctttctagcttttgattactgtgctagtgctaggtaattccaagagaagaaagagctactaattttttaatttttaaaggaatccacttttacatatttgggagtagcattaagttctgtttttgaaattgtgtttgcttcaagaaggatttttggcagtttctaaaatgaagtgagaacaacaaaataaactacttaactagaaactatcaatttgttatattatttcctatttttatacattaattcatgagcatttcattaatgtgtagttcatagataagattattgatattttatcatattatgtatggtagtatgcagaatacattttattctagctgagcttttacgcatagtccattaatgcaagttatataaattcatttgattgggaaaatattaatcttcctcaatttaaaatgcccaagtaccatcctaaaccaaaatttaaaattgtgttttcttaagttgactgacattttgattttggggtggtagacataaatgaacaaatttcgaaaacttttgttacttaatgatgaaagggtaattttatcctttgttttattgtagatcttatgccttcattcatccggcatggtccaacaattccaagacgaactgatatctgtcttccagattcaagccctaatgccttttcaacttctggagatgtagtttcaagaaaccagagtttccttagaactccaattcaaagaacacctcatgaaataatgagaagagaaagcaacagattatctgcaccttcttatcttgccagaagtctagcagatgtccctagagagtatggttcttctcagtcatttgtaacggaagttagttttgctgttgaaaatggagactctggttcccgatattattattcagacaatttttttgatggtcagagaaagcggccacttggagatcgtgcacatgaagactacagatattatgaatacaaccatgatctcttccaaagaatgccacagaatcaggggaggcatgcttcaggtagcttaacattataaataatatagtagtttatatttattgacatgtcaattaaaaagatgttcttttctagattttcttgggacacgaacaaatactactagcgaaatgtttttccactttgctcatcctgcttaagcctgtgtgcaaatagttgttaatctacactgaatggaagagtccttgtgaccatttattttattaactcatgttatgtaaacttggtatttatctcaaggctgtctagactcttaatgctgtgacccttgaaaacctagagaatttaatgtctccagttgggctgaatttaggtttaggtttggtagaataatgggaagggtaagtcttcagagacacttgacggagtcctcagagacacttgacaaacttgggaaaggaaagaagagtaggaacaactgggcatgctcagtgataccctgagcaaccacattacttcgctagatcctgccctttttctcaacaaaaagtagttcaagaattatttcctgagttgatcaaaaagacttattgtcatatggttaattatgtgttgagcatgtctttttattatttttgtaacagacaaaaaaaattaccatataatttaagaaaagaaggtactttccatatctcctgagttggagatacttctgaaaggagcagaagagatgggagaaatgtacaattctactttattcctgaagtcctggttttaccagataaaatgaagtgagatgaaaattaaactttcttgtctgattttttttttctaccaagttatagactttaaattttggcaggataaaaataaaatgtgagacagatttactttttggctcacataacttcaaataaggcgacttaaaaatggtttggagcattagtattttgactagaaaaaatttattcatttaatattaactgtttatgtgtctagcgttttccttgagtctgttggtctctgctttctggtaccttatttgtctagtaccttatttgtggaaacaaaaccaatctatacacaatggtcacagaaaaattgttatactttgtgatactaactgtaagtgcaacagggaatcagaagagagatgagtataggctggaatacttaaagagatgattcatggaagaggtaggacttggagtgggccttgagcttggcctttagattaacaagggccagaagaaggacattttacgtggaaaattgggaatagcacaatgaaaacagagtaggcatggtttaaatataaggaacagtgtagttctgcctggtttgaagaaataagatgtaaggttgagtggagatttgtggttcaacaataggcttagatttgagtcaagtggaagctcttgcacatttcggaaagacaagtgattttatgaaagcagtgtttaaagattagttttgtactagaattaagggtagattcctagagaaaagaggattgcaggtaccagtgggtaccagtggtaataggaaaacgcaaatctaggatgcttttaaaggaaaaatgagaattagagtttaggagaaagcagatcactgaagttttaaaaataatgtggaatagttggtaagaacaacttattttatgaaagctgacatcagagtctcactggaacattgtagaggagaggttttctaggcatgtggaaatgcagtaagaggcatgggtaaaagcaccttttagtttttaaggaattaggttaaagaattagctgcatgcttgagtgtttattttggttatttctgatatgcagttctgaacctcaggaataatttgttatctaactaaaatagtaagttttaaaaataaagctcatttatattttcttctagaataataactgcttattacaggaaatttgcaaaatgcagaaacgtaaaggcaaagtacctgtccaccagagatcctcttagtatgttattttcttcatctttttccatgccaaattctttttttacatggatgtgattatataatattttgtacagttttctgttctttttacttgaatgattttcttcatgttgtatactttttatgagtggttttaatgactgcgtaatcttccatttggtgcatattcagtggttaatgaggacttgacctatacttcagaaatttggaaagaggagaagtgggacacgcccacattaatgggacttggccactaacggggtttgaaggacaaaggagagggaagagtcagataggattagcacagcacatgactgcctaagagatgctcacaaaaataaatggctgaatgagtgaatgaacccagcggttttaaatctggaggcttaagacagtttttggtagctgagggagaacagaaatagctatgtttactgttaatatagaatatgtgttattctttgcgttcacctttcccacctatattgtagacgtcactgatagcttgtaatacttttctacctgaggtttaagtatgtaaatatgaggaattaataacaagttaaactcttgttttgaaaatctaatttgccaaatataattgaggaaatactataagaactaaatctctactgatcctctcttaggagcaatttgagataatttttctaagtttaaagacagactttaaagcaactttccagtcacaacttcaaaataaagtacaagttaattgcatacgttttcagattcaagtatgtcattaaaaaaataagatttcaggctgggcacggtggctcacgcctgtaatcccagcactttgggaggccaaggagggtggatcacgaggtcaggagattgaggccatcctggctaacacggtgaaaccctgtctctactaaaaataaaaaaattagccgggcgtggtggcaggcgcctgtagtcccagctacttgggaggctgaggcaggagaatggtgtgaacccgggaggcggagcttgcagtgagctgagattgcgccactgcactccagcctgggcaacagagtgagactccgtctcaaaataaataaataaataaataaataacaagatttcaaaacactgatgaaagaaattgaagagcacaaaaacaaatggaaagacatcccatgctcatgggtcagaagaattaacatcattaaaatgaccatgttgctcaaagtaatctacagattgaatgcaatccctattaaaataccagtcatttttcacagaaataaaacaatcctaaaatttccacagaaccaacaaggagcccaaatacccaaagtaatcctgagcaaaccgaacaaagctagaagcatcacactacctgtcttcgaaataaggctatagtaaccacatacaaggttatagtaaccacaaataaggctatagtaaccacaataatatggtattggtatagaaagacacatagaccaatggaacaaaaataggtaatgcagaaataaatccacatatttacagccaactgatttttttacaaaagcaccaagaacgtgcattgggaaaaggacactttcttcaataaacgatgctgggaaaattggatatccacatgcagaagaatgaaatctgacccttgtctcaccatatgcaaattcaacttgagatggattaaagacttaagacctgaaattataaaagtactagaagaaaatgtaggggaaacacttcaggacatcgatctaacaaagattttatggctaagacctcaaaagcacaggcaacaaaaacaaatagacatatgggactatattaaaccaaaaggcgtctgcacagtaaaggaaacaacagaatgaagagacaacatattgaataggagaaagtgtttgcaaaagattcatccagcaagggactaatatccagaatatgcaaggaagccaacagtaagaaaaacaaccccattaaaaagtgggcaaaggacatgaatagacgtttctcaaaagaagacatacaaatggccaaaaggtatgtgaaaaaatgtgcaacatcactaatcgtcaaggaaatgtaagtgaaaaccacagtgaggtgtcttaccccagtcagaatggctattgttaaaaagacaaaaaaatatactactggtgagagtgtgaagaaaaggcaactgttacagactgttggtgggaatgttacaataattaggacaggcactatgaaaaacagtttggaaatttctcagaaaaccgaaaacagaactaccatatgatccggcagttccactgctgggtatctatctaaaggaaaagaaatcagtatatcaaagaaatacctgcactcccatgtttatcatgtcatttgcagcagcttgaatggaactggaggtcattatattaagggaaatcaggcacagaaagagaagtgtctcatgttcttactcacatgtgggagcgaaacacattgatttcatggagatagaatgatagcagaggttgggaaggatgggaggggaggtaaagagaggtaggttaatgggtccaaacataaagttagatggaataagttctaaggtttgatagcagagtagggtaactacagttaacattttgaaatagctaaaagagaggacttgaaatgttcccaacacatagaaatgataaatactcacggtgatggataccctaaataccctgacttgattatttcatactctttgcatgtatcacaatatcacatgtaccccataaacatgtgcaaataattatgtatccaaaaaatgctataaaacaatgttaccacctgactgggctcttgtctgctatccgaatagagcagaacactgagacagcagatactgtaaagaagaaagagtttaattattgcaaggcagctgagtaaggaggatgggaaatgtttctcagagtcatcaccgccaagactggagaagctaagatatttaaagatgatttggcaggcagggggctaggggatgggtgctggtgattggactggtatgagatcatagggttatcaatgctgtcttcatgtgctgaatcagttgctgggaaattggggagtcacaagaccaagtcagttccttggttgagttcatggaacactggtccatttggtgtcagttggtccacttgtaagcaaagtctgaaaaatatctcgaagaccagtcttaggttttacagtagtgtttgtgactgccagttactatggaaagaaagctgggaaaaaatggcaggttactgtttaaacctatagcttagcagaagagtttgcaggaggtgagatcaccagtgatcaaagtggcctggcctgtttggccagtccaaattctggaaactatcaaggggggtctgtgtgatctaggatcattgntntcttggtcttctcttctattttctttttgagacaggatcttgctctgttgcccgggctgtagtgcagtagcatgatcacagctcactgcagcctcgacctcgggctcaagcagtcctcccacctcagcctcctgagtagctgggactacaggcatgcaccaccacacctggctaattttttgtatttttagtagagactgaatttcagtgttgcccaggctggtcaaactcccaagctcaagcaatctgcctgccttggcctcccaaagtgctgggattacaggcatgtgccacagcacccagtctggattattgttctttaaaagaaacaggtttgttaatcttatgggcagcctgcccaggggctgccacaggaagacaattattagtgactgccatttgttgaaatgactttgcaagcagtcatacatggaggtggaaaaagagaaggaaaatattttaccaaaattcgggcccctaccgtaattttaatcttgagggtgtttttaattttatgagggaggcttcagcaggagtcagcaagctacacccataggccagatagacttgcctcctgcaatacagtagttttattggaacacacatgcactgaaataagatttgatttcatggaatagccaagaccttgttttaatgagacagtagacttctgccttcgaagtctcatcccaggctcttttatatcagattgcgttaggtttaaagaaggttgcaggtagttgaagaatctcaggtaataaggtatctctcccaaccaaaaaaaaaaaaaaaaaaattctttacagcagttgatacactgcttgagagcttgagagacttcatttgataagattaatttttatcagggcaatatagatttaatgttttaccttgtagaaaaaagtagtactttttttgaaggaatctctaaagttgcagggaaagattaataaccaattacatgccaaacagaaaagcctaataactgcctcttccacaagggaaaaatctgaatatgagacagtagctgaaagtttcttgaattcaccttgagaagacttaaaacatatttttaggatttatgctgaataaggttcaaggaatttgaatgaatagaaaggagaaagtaagtttgtaggaggataggataggtttgtgcgatagatctattttcagaaacattgaattttgattgtcataagcataaaaaggttggaaaatttgttttgtaatgacttaatggcaatagtaattcctgtcaattagttattagattttaaggaaacaccttgtgtcaagggttaaattcagcttattgtatttagtagaagcacaaaaacaagttgtataatctcagaaagttttagagcctagaaagttctacgtttagctgtagaactttcagattagaaaatttgacctttaatgttcttggccgcataccagtcacgcacttactgtatgttccaaagaaaggaatcctgctgttaacaggaagtctttagttggaatactaaaggcagactgaattcctttgttttgcgcttaaattttagttctgtctcgaaaacatttttaaacgtttaaagatgactaaagaaatcaggatacaaaaatcagactgaaaagtgtgagctttatcaagaaatactggatttgcagttactcgggtaatgtgtaaatatttgtgtaaccagaatcttttgaggaggggttgataatctttagagacaccaccttttctgtatttattttgcagaggaagagagaaagcatttggttgagtttaatacttagatctttttatcaacatcttgattataacacatgctaataatgtgtagtatccattactagggaccctgtggaactgtgttagagctctagatccattttattctctgtagccactacaaaatttggaaaccggcctatttgagtcttattcataatggtagatctcataaatatctgcctggttgtatgagattaagttttttggcacatctactgatagctgagagagaaaataatactttatctgtgtcttcgactaagtcatatgtttagtgtgtgaatgttttactttgtattaatagaaggtgtttgtgcagttaattcctgcaaatagaagctgttttgaggattaattgtgaatccagtttattactttcaaggactacttgaaattacagaaaggtaattttgttcttgtcactcaaaaactgaagttatcattttttcccactgatttggatcaaaattggtggcttgctttttcttttactagtggtagtcatatttttctttaaagaataaataacaattgatcttttgttgctgagattacatggttagtaacaggtttgcatttgaccttataccacttgttgtgtgaccactggtaaaataattcacttgaacctgttttctcatctgtaaattggtggtaatagtactacctcctagagcagttttggctatgaaataagacatatggaaagagctggggatagaatacacatccataactgtttgcacatgtcctttgtaagagtgagtatttcccagcactttgggaggccgaggcaggtggatcacaaggtcaggagttcaagaccagcctgaccaacatggtgaaaccccgtctctactaaaaatacaaaaattagctgggcatggtggtgtgtgcctgtaatcccagctactcaggagtctgaggcaggagaatcgcttgaaccagggaacccgcggtgtggagggtgcagtgagccaagattgtgccactgccctccagcatgggcgacagcgtgagactccgtctcaaaaaaaaaaaaaaaaaaagtgagtgttttgtgttctgtgcttttcccacatacttgggatgttagttattgaagtttgagctttctggctgaactcatactggaggaatgatttattagagcttgacatagcttttttattttttaatcttctggatctgttttgataatgctttatgctttagaacagtggtttgcattgttttgtactgtacatttatctcaatagattttgtaaaattatcccaaataataatttgaaaaaatatatcacacattgtgctttcagaaagtacagctggggccgggcacggtggctcacgcctgtaatcccagcactttgggaggctgagacgggcggatcatgaggtcaggagatcgagaccatcctggctaacacagtgaaaccccgtctctactaaaaatacaaaaaattagccaggtgtggtggcgggtgcctgtagtcccagctactcaggaggctgaggcaggagaatggcatgaacccaggaggcagagcttgcagtgagcggagatcgcgccaccgcactccaacctgggcgacaagagtgagactccatctcaaaaaaaaaaaaaaaaaatacagttggggtatgttttgaaattattaatagatatctctaagaaagtttannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttgaaagggtaccgaacaagggtgatctacttgttcttgcatctgtttttgttcttgcatcctttttgccagaatactgcatagataatgttgtgtactttctattgcataggttccagtgacatataatgactgatttgtctgtgatgttaagatgcagccatgggtacagatgtcagcttaattcatccatcatcatgaagttctccataagccttttatctattattggtttttggcagccattaatgatcattggctagcactattacaggcatacctcattttattgtgctttgctctattgtgtttcgcagatactgtgttttttacaaattgaaggtttgtggcagtccaacagcacatgctcatttcatgtctctgtgtcacatcttggtaattcttgcaatatttcaaacttttttcatttctgcctgttatggtgatctgtgatcagtgattgctcatgttactattgtaattgttttgggacatcacaaggaattgcacttgtgtgcaaactaaactaaaataagaagtggagcctgatgatgtgactgcattgctgctggtctcatgataaaacttgaacagatgagttgctttttatggatgagcaaagaaagtggtttcttgagatagaatatactcctggtgaagaagctgtaaatattgttgaaatgactaatgatttaggatattaaataaatttaattgataaagtagcagtggaatttgagaggattgaggccagtttgaaagaaatgctactgtgggtaaaatgctgtcaaacagcatcacatgctacagaaaaatctttcatgaaaggtaaagtccattgatggagcaaacttcattgttgtttttaagaaattgccacagctatcccagccttcaaacaccaccagcctgattagtcagcagccatcaatatggagacgggaccctccactagcaaaaagatgctgtgaactcactaaaggctcagataatcattagcatttttgtagcaatacaattttttttattaagacctgtactttttttaaagatataatgctattgcacacttaatagactgcaatatattgtaaaatataacttttttttttttttgagatggagtctcactctgttgccaggttggagtgcagtggcatgatctcggctcactgccacccccacttcccaggttcaagcaattctgctgcctcagcctcccaagtagctgggactacaagtatgcgccaccaggcccagctaatttttgtatttttagtagagacagggtttcaccatgttggccaggatggtctcgatctcttgacctcgtgatacacctgcctcggcctcccaaagtgctgggattacaggtgtgagccaccgtgtccggccgtaaagataactttaaggttcactgggaaacaaacaaattcacgtaactcgctttattgcaatattcactttattgcagtaatctggaaccaaacctgaaatatctccaaagtatgtctaagtgttgcaaattttgatacttgaattctgtcatttcttctttattagttggaatattataattaaggactttatcaattatttggttgctgttcatataggaaagaagggttgaatgctagatcttttcctttgtttgctatttcagaatgatgaattgataccctagccacctacaaaagtaaccatgaactcatggatttttaacgccaagctttatttggattttatcatttttttattaatgtttattttctgttctaggattcattccagagtgtactgcattgcatttcattgttgtatgtctttagtcttatttaatctatgctagtttctcagtcttttgttttttatgaccttgacagttttgaggagtacagaatgttttgtagattgtacctcaatttcaattcatttttttttcaatttctttttttttagacgtctccctctgtcacccaggctggagtgcagtggcacaatcttggctcattgcaacctccacctcccgggttcaagcagttctcctgcctcagcctcctgaatagctgggatcactgccatgtgccaccacgcctggctaatttttgtatattcagtagagatggagtttcgccatgttggccaggcaggtcttgaactcttgacctcaggtgatgcacccgccttggcctcccaaagtgttgggattataggcgtgagccactgtgcccagccttttttttcaatttcaaaagtctgatgtttttctcatgttccatattatcatgtattttccctgacccagaagccgtttctccaagatgagctggttgattttaagatttttatacttagttttaattattggtgtggtaattccttttgatgtttaaattgctgctgctttggtggcaggagccctttcaatgttggttcctttttatttctctttgctttctaatgtgactagatgttccaggctcatattgtgcattttctgcctcagtcctgtactcaaccattctaaggatccctggttcctttcgatggggaaatagtattcagggattgcactcttgtactcattgctatttggtggattaatgcttgtagaccttttcaatggacatatctgggaaatatgttttgttgctttctggaaatagaaattcattatgagttcatactgttggcagtttaagatttccaagtttttacttttagctttttaaaaaaatgtttaattgtgataaaatacacataacaacatttaccatcttaaccatttttaagtgtacagtttgttggcagtaagtacattcacattgaactaccttctttttgccttaggtttttatttttttctcatactgaaaatcttggttcctgttgacaattatgtaactattcatttgctttatctgtagcataactagctttaaaaatagtatcatcgacaatatgattactgaataaaacagaagatttctttgtagtttattttttaaatttaggatatttcccgatgaggaagtacaatttattctctttgggccgggcacaggggcgcatgcctgtaatcctagcacactgggaggctgtgatgggaggaatgcttgagtctaagggttttgagaccagcctgggcaacatagtgagaccctgtctttacaaaaaataataaaagaaattagccaggcttggtggtgcatgcccatagtcacacctacttgggaggctgaggtgggaggattgcttgagcccaggaggttgagactgcagtgagccgtaattgtgccactgcactccagcctgggtgacagagtgagaccctgtctcaaaaataaataacaataaataagtaatacataaaagtcactttgaattaatttttctgaagtatgctttagctgcttcatgtactcttagatttgtttacttcattttgttttcactttttagggattgctttttctaaattttgatttaattttgttgtaagttacataggatatttaaaaggttgcaaaatcaaaatgataaaacagaatgcatttagaaaattaattctacccctgtcctcttccaccccatttccttcctccccctgtaaatacccaattttattaatttttgttacatatttcattgcaacactttatgaaaatagatacaaattacatatatatgtaaaatatgtattcatattattctctttcatatgtattgtgttaggtttctgtagctgtaaaacacattacttccaaactcattggcataaaacaataaacatttattatctcagtttctgtggttcaggattttggaagcggcttagccaggtcattctgactcaggatctctcatgaagttggagttcacacacgtcagctggggctgcagttatccgaaggcttcgctctggctgaaagatccatttctttctttctttttttttttttggagacagagtctctctctgttgctcaggcttgagtgcagtggcgcgctctcggctcactgcatcctccatctcccaggttcaagtgttctcctgcctcagcctcctgagtagctgggactacaggcatgtgccgccacgcccagctaattatttgtaattttagtagagatagggttttgtcgtcttggcccggctggtctcaaacttctgacatcaggtggtccatccaccttggcctcccaaagtgctgggattacaggtgtgagccactgcgcccggccaaaagatccgcttctaagttcactcacaaggctgttaacaggaggcttcagttctggctgaaggcctcaattcctcactcatgggcttctcatgatatggtatctggtttcccccagggtgagtgatgagagagcaagtacacttgaccaaggcagaagctgcacggtcttttatatcctaatctcagaagtcacacaccatcctttctgccataaactattggtcacacagacaagggtgtgaatcccaggaggacagggataattggggcccaggttcaccttactggctggctactacatatatgtatatatgctggtgtactgtccagactcttttttcacataatacatcccgaatattattccagggcaataaatagaaatcatcttcattcatgtttacagctatataatatagtactctgttctgtgcgtgtaatagtgtaatcatccagtttgctattattggacatttggttgatttctagccttttgcaactatagatgttgctgcagtgactagccttatgcatatgcattgtatgttgtatttaaataaaaacagtttcttaaaccacatgccaataacccatatttatccatacacttacatatccttctttagaatctgagtttgaattgaagcatttaccttaattttaaagtttcttctaaattaccttttgtcactggggtctgtttcatttgcctttctttaaaggtaaacttgatgttttcattttgccttagtttccagccaactgtaattttaaaagctcttcaaagtcagctgggaacttttgactagtagatgttttgctctggcatggtactccttcacacctcaatggtcacccagtctttcttattttgaccattctgagatattctaaatttggcagttagggccttgggttgactggcagtcttctgttaacacactaacttttcatttaaatgctgtatccttgtacctttcaggcacagggattcaaatttatgttaaaattttgtctggggtgcaactgttacaaataatgaggattaataatagtatgaaccacatactgtcttcctaggactgtgttagaacatgcaccagcaatgataatacatctgtatttgctgcctagtggctaacagatttactctcttgattataaaaaacaccttcgtttcagaactagtaaaatatgggagtgaaagaggcaaaataagtccaaaaatcaaggaaaatatttatgcagtttctgtctcattgatgtgtgtatctcctccactaatactacactgtctttattactgcagcttatttatttaacacttactatgtgctttattctgtagacactgttctagagactggagatgtatctgtgaacaaaacaaagattcctgtcctaagaatcttctttgtgggattcttgtaggtaaataaacaaactaaaattacataagttaaaaggttaaagtgctttttaaaaaagtaaaacggtaagtagaagctcagcagatgtagactgccatagtaagaataagcttcattgaaacatttgagaagaggcttgaagagggtgagagtgagccaagagaatatctgaaggaagaatcttctagggagaataaacagccagtacctgccgtgttcgagaacagcatccaggaggtgagtgtagctggaacagagtgagctaagggtaaaaaaaatagcagcttaagtcagagaagaaatggaggtctgatcatcagtagggcttcatattgaaagttatacaagagataactttcatcaagtaatgagacagtaaagataacttgtaccaaatatcaaggaaaaaacttactgagctcagccagtatcaccaacctgtggtgaaccaaataaatacttcttgttttaagccactgagtttcggggtgatttgttatacatatgctattagtattctttttttttttgagacggagtcttgctccgtcgcccaggctggagtgcagtggcgcaatctcggctcactgcaacctccgcctcccgggttcacgccattctcctgtctcagcctcccaagtagctgggactacaggcgcccgccaccacgcccagctaatttttgtatttttagtagagatggggtttcaccgtgttagccaggatggtctcgatctcctgacctcatgatctgcccgcctcggcctcccaaagtgctgggattacaggcatgagccaccgcacccggccaactattagtattcttgtgtgagaataaaattatgcagccagattattcttttaaaaaatatagtaaatttaacttttttcaagttacacagttctgagtattaacatatgtatagatttcatgtaatgcctgtgtaatgagaatacagaacatttcaaacaccctctaaaattcccttaggttgtcctcttatagtcaactcctgtccctcacctccagttcctagcaatcactgttttctgtcctagtagttttgtgtttggaagaatctcataaaaatggaactgtacaatttgtagccacttgagactggctgctttccctcagcatgatacttctgagattcatctttgagattcttgcatgtaccaataattagttccattttattcctgagtagatttcactgtacagttgcaccacagtttgtttatccattctgttaaaggacatgtgtgtcatttccactttggggcaaatatgaatacagtcattgtaaatatccacaaataggattttgtgtgaagatgacttctcatttctctagggtaaatatttgggaatgggattgctgtagcatatgaaaagcatatgtttaactttataagaaattgctaagctgttctccagagtgactgtagcattttgcatccacaccagcagtgtatgggcattccaattgttctgtatccatgtaagcacttgcattgtcaggttttgtttggttttttggccattttcatagacgtgtattgtcatttcattatggttttaatttgcatttctctcacggctaattatgatcctgagcattttttcacgtgctatttaccatcaatatgttgtcttcggtgaagtgcctgttaaaaattcttaccccccttttttttaactgggtcgttttgttttcttcttctgtttttgttttgagagaaggggtttcactctgttgcacagaccggagtgccgtagctgttcacaggcatgatgatagtgtgctacagacctgaaatcttgggctcaagtaaccctcctactcagcttcctgagtaggttgaactacaggtgcgtgtcactgcagcaggctcagggttgttttcttaatgttgagttttgagaattctttgtatattctaggtcagagttctttggctaataatgtggtttgcaaacattttctccctgtttacagcttgattttctttctcttagtgtccttcacaagcgtttttaatcttgacagagttcaatttaatcagtattttttctttatgagttgaactttcagcatcatggtaaaaaaaagtactctttgtctaaactaaactcatgaaaatttattctatttttttcttgtaaaagcgttatagttttaaatttagatatatgatctatttttagttaatttgtatgtaaggtggcaggtataggttgtgattaggttttttgcatatggatgtccagttgttgcaacatcatttgttggaaaaacctttccccgctattgccctttcacctttataaaaaaatcagttgaccatgtctgtgtgggtgttcttctggattctctgtttggttccatttatctatttgtctgttcttctgacaataccatactgtcttgattaatgtagctttatagtaagccttaaaaatcaggtagtgtgagtccttcaactttatccctttttcaaaaaaattgttttggctactctagttctcttttgcctttcagtatacattttaaaatctgtttaataactatctgtatctacaaaaactccatggaattttttttttttttttttttttgagacggagtttcactctcgtcgcccaggatggagtgtaatggcgcgttctcagctcactgcaacctctgctctcgggttcaagcaattctcctgcctcagcctcccgagtagctgggattacaggcgtccgccaccacgcccggctaatttttgtgtttttaatagagacggggtttcaccatgttggtcaggctggtctcaaactccggacctcaggtgatccgctcaccttggcctcccaaagtgctcggattacaggcgtgagccactgcgctcggccggaatttttattggaatgacagtagtctcgctttatccatggttttgctttccacagtttcagttacctctgcaactgcagtctgaaaattgatgaatatggtacaagatctttcaagagagagggagagagattacattcacccagtttttattataataattgttcaattttatttttgttaatcccttactgtgcctaatttataagttaaacttcatcatagatacgtatgtataggaaaatacacagcatacacagggttaggtactatctgcagtttcaggcatctacggggggtcttggaatggatcccccatggataagggggggactcctataaagataaagataatttcaggagaattgacatcttaactttatgtcgaatcttccaatccgttaatgtgataggttttttatttctttcatcagtgttttgtagtttttagcatattggtcctgcacaattctttggagctattataaattatattatctcaataacttttgtttccagttgttgtttgttaatatataaatattaatttttgtatggtgaccttaaccctgctaccttaagcttacttaataattctagggcacagccagattacttttaagaatttataaccgggaatggttttacaagttcatttttgcattttttttaaagctgaaatgtcaaatctttaatcgggaaattttctttttcccacttttcttttggcagtatacatacttaagtagctttactgctacaggttgattatcccttaccccaaaatgcttaggaccagaagtgtttgagattttggatttttttttttattttggaatatttgtatgtacataataagatatcttgggaagagatcccaaatctgaacatgaaattcatttatatttcatatataccttctacacatagcctgaaggtaattttacataatattttaaataattttgtgcataaaacaaagttttgactgaattttgactatgactcatcacataaggtcaggtgtggaattttctgcttgtggcaacatgtcagctctcgaagttttggattttggagcatttcaaatttcagatttttggattagggatgctcaacctgttgttaagtgtttgtatggcctgttgatggaagcatcagccaaaaaaccctctactaatttcaaagtctagttcctccccctttttataattccttaatagtaatactgactagttttatgaaaaccatcataatgggcataatcatgaatttaaatagaagagtacaatcaggatagttaatgttgtgactttgctagctggcacattttttgcattgaataattgttctgtttcttctctatcattttctctttccccgtaggtattgggagagttgctgctacatctttaggaaatttgactaaccatggttctgaagatttaccccttcctcctggctggtctgtggactggacaatgagagggagaaaatattatatagatcataacacaaatacaactcactggagccatcctcttgagcgagaaggacttcctcctggatgggaacgagttgagtcatccgaatttggaacctattatgtagatcacacaaataagaaggcccaatacaggcatccctgtgctcctaggtacagtatcaactttattttcaactttttgtttcgggattgtctaatgttaggttttgaactgatattttcatctaagagaatagcatcctaaagcttgagatttgaaagccttatatttcaaaatttacatttttctctggacatacatttagtaataaatgactttaataagttaaaaaaatacttttctctttaaaaaaacctcaaaaacaagatcattgttcttatgatcagattctgctgtagtaatgccagaatgtttttgttataggtgtccagaaaattaaataattaaagcttctgccttcttacacttaaatttttacaaataataacagacctaccccctaagtcagaatgtacaattgaattataacattttgctgtttattctatttcagacatgattgtatgttctgtttcttgctattactagacaagaaagtgaaaattgggactgaacaaaacatcccatttctgtttaataaatatagccagtaattctttgtgctgttttgattgatactgggctgggtagatctgctttgttgccagacttttagccctgtgctgcctgatagccagttgtcacgtgtggctgttgagcccttgaatgtgattaatataaattgagatgtgctgtaagtgtaaatacacactggatttcaaagtacataaaagctagtattagtttagtaatgaagttgcaaatatttcaaattaaccacatttgagcttcaaaagctgagccacaagattaaaaatgagtaaaatatcatctgcattttgataaatgttgcttagtgtcatctggcataatacagttttttggttatagtggacatttgtattttcttagaaccattaaaatagttatgaaaatgtgttttaggattatcaactggaaaactaagctttatcattattgtgttgttttaattattttatgtatttggatttattaattcaactttgaaaatctgttttttcccatgattccatgtagtggcacccaatcagtgtttattcaactactgaggttgttgtcctttgaaagaatcaaacctgtggtgtggtcatcaggtggcaatgattgttgcagtccagataaccagatttgagagtttacttttctgttcgttgacatggtttggctctgtgtcctcacccaaatctcatctcaaatcgtaatccccatgtatcaagggagggacctggtgggaagtgattgaatcatgggggccatttcccccatgttgttctggtagtgagttctcatgagatctgatagtttaaaagtgtgtggcagttcccccttctttctctctcctgccaccacgtaagatgtgccttgcttccccttcatcttctgccatgatttaagtttcctgaggcctcccaattatggaactgtgggtcaattaaacctcttttctttataaattacccagtttcaggtaattctttataacattgtgaaaatagactaatacaatcatcattaaccttatgttattacagaattttaatagttttttaaaagcaatttcataattagttaatttacttctctcagcaacttggtagaagttgaacaagagagaataaacgctgcccttaagctcacatatgtagtattactagttgtctggttgtctatcttaggccaatccttatacaaaattcttgacttacattaggtcatttaatcctgagaacaaccacctgtggtatgaatttattattccacttttacaagtatggaggctgaggcttagggataggaggagttttgcttacatttacatagtttgtaagtgttaaagctgtggttcaaatccaggcgtacctgacttcaaagtcccaattcttcttcacccccagttgtactatcaaagaacattttcctttgaacctatgtaggaaagaagagttgaagatagaattcagttccatttgcatattgatcttttaaaattgttaaccacagcataatctaaccttattttaatgccaggttagtaacagtgaccttttctaatatacacatgcatttttttctccccattgttatatatacaccacattgtcttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaggtggtgtaagtcagtttggacagaaggcacactgaaaaatgcttaagtattatttgttctttataatttcttgtttagattttaagacctttatcntaaagaatgtcactcctttcttcttagtgtacctcggtatgatcaaccacctcctgtcacataccagccacagcaaactgaaagaaatcagtcccttctggtacctgcaaatccatatcatactgcagaaattcctgactggcttcaggtttacgcacgagcccctgtgaagtaagtacatttacagcacaaatttgtttgaggagtgagtgaagggctgaatggagtctctgggctgctcgatccaagtagtgtgccatattttactcttaataatattcggcttctaagtaatacgtgacttgaataaaggatgtcatagtttataatgtgtgaaaatcattaattagaaatattagttcttcagacatttatagaatcttcatttctttgtgtgcgggcctctgctattagggatacaagtaagaaacttacattctcataggagaagattaatttgtaaataagtgatacagtacttgtgataaaagtaaggacctgggcttggcacagtggctcacacctgtaatcccagcacttcgggaggctgaggcaggaggatctcttaagtccaggagttcgagaccagtctaggcaacataacgagagccccatttccacagaaaaattttaaaaattagccaggctttttgcatggtctgtagtcccaagtacttgggaggctgaggtgggaggatagcttgatctgaggagttcaagttcagcctgagcaacatagcaggactctgtctctataaaaaatttaaaaattagcagcatgctgtggtgtatgcctgtgatctcagctacttgggaggctaaggtgggaggactgcttgagcccaggagtttgaggccgcagtgagctatgcttgtgccactgcactccagcctgggtgacagagcatgagagactctgactgcccccctactagtgccagaaaaaggaatgatagcagatggcattttctttcctattttcctcttttctttgtcccttgataccacctgacctacagctgtatgtttgtatgctgattctaaatagtacataatatctgtatgcgtttttattttccttaaatctggaagaatatgtgcctaactgttaaccttagtatttctgggggtggatatggttgaggagagttattctttctgttgtatgttatttctactctatagtcatgtttaataagaaatagcataatctttcctttttgggggaagagatagtacttaaatggaattcctggtttcttacagaaagaaacagattcctgaagattatagtctttttgaattctgtggttctgtgagctaactataaaactcaaggaaactagcaaataggttgtaattcttccaaaatgggccagactgacaaaaagggagcaagtactatacttctttactgcttaaaaacttaaacaagaggtctggaagcctgttgctttctagttgattctaattaatgttgctgaagaggatggaaatggttaatattctatcaacaaagggtgagtggttatctatgaattagataatgaagaatcaattatcacaacaaaaagagacatttttgcaaaaaaaatttgcatgtttcttccttttgtttttcagacaattgagaatctgaaattctttaatcctgaaattctttaatcctatctctcacagtaatttaccaacacaatttttgcatatacttctaaaacttgatttagaaacagactgtgttttaaatttgtatttaatttttgtaaagttatgttcctgtgtctgctcacaaaagtctataatgcttggtataactgtactcaagctgaactataggataaaattttaaaagttaggccgggtgcggcggctcatgcctgtaatcccagccctttgggaggctgaggcgagtggatcacaaggtcgagagatcgagaccatcctggctaacacagtgaaactctgtatctactaaaaatacaaaaaattagctgggtatggtggcacgcgcctgtagtcccagctactcgggaggctgaggcaggagaatcatttgaacccgggaggcggaggttgcagtgagccaagattgcgccattgcactccagcctgggtgacagagcgagactccatctcaaaataaaatataataaaataaaatttaatctttgacagcaaaaacattgtcaagaacatatttactttctgttatttctcacacacacaccaaaacactagtcctcagcagtgtgtggcaggggtattttttattccagtggcttggcagtcctgcagtgtcaccattgaccctgctctggatacaggtgaaaaagagggaggagtggacacagtgttatggcgggtgggtgcaggggacttaggctacatggtgatacctggagctgaggatagcagcactgttgatcttgaagtcagcatgcagtgtggacaggttatttttggtagcattatagcaactggcatttgtatatatggcttatttataagtagtatttgaatagaatactgccctaggaaaaacagagtgaaagagatgctagaagcatggactactggagtgttttacacccaattttcggaatgatttacctgtgtatatacagtataaatgactaaaagttaaacttgtggaagcaaatttcttggctagttaagaaggaaagaatgggagtttatacttttcagaaaaagaaggaacacttaggtgattataggcagaagggtagtaactatgtagaagcaacagaggaaagaatttggaataggatttccattccatgctttgcagttttttcacatctgtgcccttcttggagtgcactttcttcccccagaaggattttctctaatctgattttgttgcttctacccataatcgtactcttgatgctgaatgctgattggcaagtcagaaacttggccaggcatggtggtgcacctgtaatccaagctacttgagaggcagagtcaggattgttagagcttaggagttctgggctgtgatgcactatgccagtcggatgtctgtactctttggcatcagtatggtgacctcccaggagcagggaccaccaggttgcctaaggaggggcgaactggcccaggttggaaagggagcaggttaatactactgtgccgatcagtagtggcattgtgcttgtgattagccactgcacttcagcctgggagacataggagatcttcatctcttaaaaaaaaaaaaaaaaacaaaaaactgacaaaattgtttgaagattttttcttcctttagacaaaacttgaatacttatacagaatgatattcgtgtcttaaaaataagtataatatttaccatcttatttttgttctttgttttccttgctagaaagtacacagatgatagattagtttaacattgttgtagttcacctggtataaatggtaaccatcttgacttagtagacattgctgaattgcctcttagctaactctgggagattaagattttttgttaggaattatgaatttgtttccaaggagcacataaaatcttaagttacaagaatggtggatgttctaagaaataccttataactgtttcttcatggaggctcattgagtatttcttacagtatcactctaatttatgttcacaagtggttggagtttcccattaaaaagaacagagcttcctaccaaagaaatctaaatctattaataaatttgcagctctcaagtaactgagtgaaattggcagtccaattaaatatatttaccttccagtttttgcatgttgtattactgacatttctgttacacatactcaagttgaatagaaacatgtcaaaatgccaagtatagtggctcacactaactgctcggtatgttttctatctcccttttttaaaatctcattttaaaatattagacagtccttttgaaagaaaatggtataatatcaaagaaccactgggtaggaaatactataaacgatcattaaaaataatgctagagtttgcaatcaaagcttcctattatgtctgctctgcagtatgatttgagaggtataatttcgggtataacatgcgtttccctaattcaagttctttgtctagtcttagtggatattaacattgaaaaccactattttgaagcatcttggtccatagttctcttcctttttgaacttttagtcactaagcacttttgcaagaggtaaagagcctctttaggttttagtgtttttcatctttaaaatgagaatattaattacagtgctgtcttcatcacatactgaaatggaactatgtagccgactactaaaggtactactgctaagcagggatctacttaccactgaaagactactaatggtcttccatgatatatgtaaacagaatcttttgtgaatccttcaccacactgaagccttctatagtctactcattctctcattttaaaaagtttattctttccttctttagttttcctgtttgtttttttagatgctgtgtactcagcctcaagaatacttagagccacgaattgagggttcctcttcatttctccttctgtttggttccttgtttgctgttgtgaatagcattcctcaaaaagatgcatcaacatgaatgatgcaaatgaccatccatttttgtgcattatcatttgcaagcacaatatggtctaaagtattcttaagttcctgacagttaacttttgatgaacttataagtagcacaaaatatgttcttcagaaattatagaaccactgtagtttgaaatctgtgttcaaggttgtctggtgttaatattagaataatgggtaatttaaagaaataggtattctttaacttaaattgaaatgaagtaatattagcagttttctcctttgcttagaagaattatatgtaggccgggtgcggtggctcccaaagttaatcccagcactttgggaggctgaggtgggcgaataacgaggtcaggagattgagaacctcctggccaacatggtgaaactctgtctctactaaaaatacagaagtagctgggcatggtggcgcatgcctgtaatcccagctacttgggaggctgaggcaggagaattgcttgaacccgggaggcggaggttgcagtgagctgagatcacgccactgcactccagcctggtgacatagcaagactctgtctcaaaaaaacaaaccaacaaagaattatatgtaaatagcagttaataactcagatttttaaaaaactgatttatttatttttagaaacagggtcttgctatgttgcccaggctggcctcaaactcttgggcacaagcagtcctcccacctcagcctcccacctcagcctcccaagtagctgggactataataggcgccaccactgtgcccagttatttttaattaaaacttatggtttgtttagttataaataaagttgacattttaaaaaacagactagtttcttgctagaaatacatgtttatgtatgtgttgtgaacatatatatgattgtgttttaaaacagatatgaccacattctgaagtgggaactcttccagctggctgacctggatacataccagggaatgctaaagttgctcttcatgaaagaattggagcagattgttaaaatgtatgaagcatacagacaagcccttcttacagagttggaaaaccgaaagcagagacagcagtggtatgcccaacaacatggaaaaaatttttgagctgattttttaaaaatttaagttttgtaagagctttaaaatattttcacagataaaaaattgcaaacaagtactctggttaataaatgctgcttcctttgtggaaattataaaattctaactttacatgtattttgttattagaaattttcttttattgaatgagaaaaattagtctatcattttaagagccaatatggcaaacactttcaaatactgtatattaggaaactgttttggtattcttgatggaaaaaaatgcagcggaaatgtcattatgaacagatgttaaataggaaattattacttgttaacttcttacagcagtagtaccttctttaaaaaaaaaaaagaatctgcggtatttttttaaaaaaaaagtttactgctgtagtgtgaaatattgtctggaaagggatggtttaaatattaccatgatgtagttgaaatataaaataggatttggaaccttattgtgataaatatttatatatgatgtatgtttgtctcccctctccaaagtaaggaacccagctgggcgtggtggctcactcctgtaatcccagcattttgggaggccgaggtgggtggaccgcttgagtccacgagtttaagaccagcctgggcaacatggtgaagccccatctctacaaaaaatacaaaaaaattagccgggtatgatggtgtgtgcctctagtcccagctacttgggaggctgaggtcagaggatggcttgagcccaagaggcagaggttgcagggtcaagatcgcaccacttgcactgcagcctgagagagagagagccagaccttgtctcaaaaaataaaaaataaaaaaataaaaacaaaataaggaatccttttaaagcatgttgagaaagtattttttggtatgaatgggtgacgggccttttaatctttcaggaatacatataagcaggaatacttagcctgtattgggaaggtgcgtgaaactcaaagttgctaacatcatcttccttattttcagccagtgctgttactgcatttagcatggctctgacgaggcagtggagagttcctgacagcatagcatactgccttctgacagactgggttaagctaccaaaaatgacaaccagggattcaaaaaaaatttttggtcattaaatcgaatgtatattattactttttaattttctaagtatttatcacaggactttaacaccatatatttccctcccagtttttctccccttttggtttaatagttaaaacatttttcataattagtgctgtacatttgattgcaaacaaattggtgatacttctaaactataactataaatctctagaattctgtgaatttcaacattatttgaagtagttcttcagttatatttacatgtgttgtaaatatgtaaatatacttttagcttaggaagtcttccagaatacaaactactcccagttagatatcagtgagtttgaataactgaagaaatgttgacaatgttaagtgtttgattttatataacaagcttattaaatatatttttgtggaaactaagcgcgtggtttttgagtccatgttttagacatagtgctgtatttcatttttctgttcaaaaggccatagacaaaggaatcaatataattgaaaggggtaatgaaaaggggtattgaaaaatgaaagtgtggctagtggaattttatcactgggaaaagataatttgctgaacttgtgatcagacttttctaagatgtcagtatatatatcttatgaactcccacactcctctatcttaattgttctttcctgtcacttcttactttatatcgctacttaattatggtgtagccatttctgtgacagtctttagaagtttctctgttttggacctcctagataagtattacttttttcattaacacactttccttcttcctctaagaagtctgttcagtgaaactccactagagattttagtcattaagaactagaagttacctaaaggaattagcatgttttgtaaaatagaaacttaagtttgtactgatgtcaacattaaataaaatgcatcttgagtctaaaaaaagagataaaccttcatcattctatatcagcacctctcattttcaaattcagtaagcatttgtttttaatcttaggaaccagagtaaactttaataatagttcacatattaaaagaataaacctatgaagaatacattctttgtgtaccaaatgaaaaaatatacaatgcagtgaaactcacatatacaaagtgattttttgtgcattttttatcaagaggtttatattctattaaaccatgtaaaagcaatatctggtttttgataaagcaactaaggaactctaaaatgtgcttaaaaataagattgtgatatatataagatgatacagattaaatccagcctgctgagttatgatgctgtgttttaacaaatggctaatgtgaaataatttaaaattttttctgtagaaattaaatgagcatatccaaacgtggaatatcacatgatttaattataacccagtagtagaatttagtaaattcaagcagaacacgtgtactgttatcataaatagacaaatactttataacagcatttttgaagaaaaatcaggaaattgtacatgttaacaaaatagtataaccatgcatacatacatctgcttattaaacactgaagtaaaagaactaatccagtttgcagtcttcagaggtgtttagtattttactccgttctgatggaagcactttgaatgtttgcatggagacataaaactgaatatttaattgacaattggtcatgcacctgtatttcttaaaatttgcattacatttttttcttttctgattgttcagtagattccgactttggtgtagggaaagcttgatgatacagatgtctgtgggttgctgctgcactgtaaagcttgtacaaagcctagaataaaagcatcaaaattataaaacattttattaaacacacagatgtaaaattttttcctttttagtttatcgtactgtataaaatttagcatgttgaatttgaaatcttttttaatcgatatataaattattatcttaaaattcaaaagtattttcaactcctcaatgtgtgcattaacacttttgtgtgttgacctgtgtggtgaatgttttattctcaaactgtgtttccttttaccttgcatacctcttggagcagtgagcttctactttgccccaggggtgtgctgaaaattcaaattgtgaatattagcagttttagatgaattgtaagacctcataacttctgattggcagtaactattatgcatagctaggctacttgctttctgggcttccagtgttttcctgagtggggccaattttattctccatcatttactgatacagtcatgcagctaagcacatatggtgtccttcccccagcacagtaggatccttcactttacacgctcttgccttgtttccgtgacttagctaatatcttacaaaccttattcaaaaacattacctcagcaagctttcctcatttaaaactacctcttattgttctctgtgggtttttaatgatgtttgaatcataggaatctctgtgtattgtatattgctttatataagtgttcatagatatctttgtttcctggccccgttagctagtgtctaagatttcatcccctcctctgactttcacatacttgacagtcagttacaaatgacttgtctgatttagttctggagttcgtaaagagtctttgaatatttttaaaagctgcataaaagcttgagatcctttaaatagtaatctgttcccaggaaattccattaatttttatcatttgtgagtctatacctactttctttggatctggtttttttttgtttttgtttttgtttttactttttgagttggaatctcactctctcgctcaggctggaatgcagtggtggcacgatcttggctcactgcaacctcctcccaggttcaagctattctcttgcctcagcctccccagtggctgggactacaggtgcctgccaccacacctggctaattttttgtatttttagtagagatggggcctcaccatgttagccaggctggtctcgaactcctgacctaacgtgatccgcccacctcagtctcccaaagtgctgggattacaggcgtgagccaccgcgcctggccttgattttcttatggcattacgtctgtatggtaccatcttcagatctgtgaatgtatattggtgcaacatagtattgttttccattatattctgattctaaggcattcaatttaattctaattctcttatattttttcttaagctttgatttacaaagttgaaatttgcttttgcttttttaatgaacttgcttactgaattttgcataaaataacttcttattgacttgtaaatagtaaatggtatttgattcgaggacataatccaattatatgccacagtagaaactgaggaaagaattatctctatagttgttcaagatcagctttgcttcctgaagataatgacctaaatgattccagagtggtaatttttcttagtgtaacagatgattccttcgtggatgcctagagtcttttctgagatatgtagtgttgcacatgtgcggtaggagaaatgaacactaatttacaggtcaggcctgaggtctgccaagaaagaatacat

SEQ ID NO: 7 - DNA, 25 bp, Homo sapiens
gtcgagaaggatgctgtcccgaaag

SEQ ID NO: 8 - DNA, 25 bp, Homo sapiens
ctccctctcattgtccagtccacag

SEQ ID NO: 9 - DNA, 24 bp, Homo sapiens
ggagagttgctgctacatctttag

SEQ ID NO: 10 - DNA, 23 bp, Homo sapiens
tattaaccagagtacttgtttgc

SEQ ID NO: 11 - DNA, 2212 bp, Drosophila melanogaster
cgatcgtacagcataaatttagctcgaaaatggactaaacaaagacagcgatccggaatccgagcggaaacatagtctgcatgaactatctaacgatcctgctgtgcaaccgaaaaccgacgatgctctcgcgccggaacaaggagaagtcccagcacaaggagggcgtggtggggaagtacatgaagaaggacaccccaccggatatttcggtgatcaatgtgtggagcgatcagcgggccaagaagaaatcgctgcagcgctgtgcgagcacctcgcccagctgcgagttccatccgcgcagctcgagcaccagtcggaacacctactcctgcacggactcgcagccggactactaccatgctcgacgagcacagagccagatgcccctgcagcagcactcccactcgcatcctcactctctgccccacccctcccatccgcatgtgcgtagtcatcctcccctgccgccccaccagttccgcgccagcagcaatcagttgagtcagaacagcagcaactacgttaatttcgagcagatcgagcggatgcgccgtcagcagtcgtcgccactgctgcagaccacatcatcgccggcgccgggagccggaggattccagcgcagctactccaccacccagcggcagcatcatccccatatgggtggtgacagctacgatgcagatcagggcctgctaagcgcctcctatgccaacatgttgcaactgccccagcggccacactcgcccgctcactacgccgtcccgccgcagcagcagcagcatccacagattcatcaacagcacgcctcgacgccgtttggctccacgctgcggttcgatcgagctgccatgtccatcagggagcgacagcccaggtatcagccaactagctctccgatgcagcagcaacaacaacaacaacaacagcagcagcagcagctgtagcacacacaactggcagctcacctgggcggcagctactccagcgattcgtacccgatctacgagaatccgtcccgcgtcatctcgatgcgcgccacgcagtcgcagcgatcggagtcgcccatctacagcaatacgacggcctcgtcggccacgctggccgtggttccgcagcatcatcatcagggtcacctggcggtgccatctggaagcgggggaggatccctgagcggcagcggtcgtggtggcagttctggcagtgttcgcggcgcctctacctcagtgcaatcactgtacgtcccaccgcgaactccgcccagtgcggttgccggagcgggaggcagtgccaatgggtcgctgcagaaggtaccatcacagcaatcgctcacggagcccgaggagctgcctctgccgcccggctgggccactcagtacacgctacacggtcggaaatactatattgatcacaatgcgcataccacgcactggaatcatccgttggagcgcgaaggtctgccggtgggctggcggcgggtggtgtccaagatgcatggcacctactatgagaaccagtataccgggcagagccaacgtcagcatccatgcttgacctcctactatgtctacacgacgtctgcggagccaccgaaagcgattcgaccagaggcgtcgctctatgccccacccacgcacactcacaatgcactggtgccggccaatccctatctgctcgaggagatccccaagtggttggccgtctactcggaggcggactcgtccaaggaccacctgctgcagttcaacatgtttagcctgccggagctggagggcttcgacagcatgctggtgcggctcttcaagcaggaactgggcaccatcgtgggcttctacgagcgctaccgtcgcgctttgatactcgagaagaatcgacgcgccggccagaaccagaaccaaaaccagtgacccggtgaccaggtgacgactgactcagaccacatactcgccagcagctatatgcacatcatagtgctcctgtaatcgacctttaacttatttaaccatcgactcatcgcgaaatcagtgccttatacgaaaccagacgagatggtagccaagcagatccatgacagttcgaatgccttgatgaaacgtagaattgtgctacgttctatataaccttaatgtgatttgagcttggcgtttgtttgtaatgtgagcaaagaaaattaaactggtttactgatcatcttaaaaaaaaaaaaaaaaaa

SEQ ID NO: 12 - protein, 288 aa, Drosophila melanogaster
MNYLTILLCNRKPTMLSRRNKEKSQHKEGVVGKYMKKDTPPDISVINVWSDQRAKKKSLQRCASTSPSCEFHPRSSSTSRNTYSCTDSQPDYYHARRAQSQMPLQQHSHSHPHSLPHPSHPHVRSHPPLPPHQFRASSNQLSQNSSNYVNFEQIERMRRQQSSPLLQTTSSPAPGAGGFQRSYSTTQRQHHPHMGGDSYDADQGLLSASYANMLQLPQRPHSPAHYAVPPQQQQHPQIHQQHASTPFGSTLRFDRAAMSIRERQPRYQPTSSPMQQQQQQQQQQQQQL

SEQ ID NO: 13 - DNA, 2212 bp, Drosophila melanogaster
cgatcgtacagcataaatttagctcgaaaatggactaaacaaagacagcgatccggaatccgagcggaaacatagtctgcatgaactatctaacgatcctgctgtgcaaccgaaaaccgacgatgctctcgcgccggaacaaggagaagtcccagcacaaggagggcgtggtggggaagtacatgaagaaggacaccccaccggatatttcggtgatcaatgtgtggagcgatcagcgggccaagaagaaatcgctgcagcgctgtgcgagcacctcgcccagctgcgagttccatccgcgcagctcgagcaccagtcggaacacctactcctgcacggactcgcagccggactactaccatgctcgacgagcacagagccagatgcccctgcagcagcactcccactcgcatcctcactctctgccccacccctcccatccgcatgtgcgtagtcatcctcccctgccgccccaccagttccgcgccagcagcaatcagttgagtcagaacagcagcaactacgttaatttcgagcagatcgagcggatgcgccgtcagcagtcgtcgccactgctgcagaccacatcatcgccggcgccgggagccggaggattccagcgcagctactccaccacccagcggcagcatcatccccatatgggtggtgacagctacgatgcagatcagggcctgctaagcgcctcctatgccaacatgttgcaactgccccagcggccacactcgcccgctcactacgccgtcccgccgtagcagcagcagcatccacagattcatcaacagcacgcctcgacgccgtttggctccacgctgcggttcgatcgagctgccatgtccatcagggagcgacagcccaggtatcagccaactagctctccgatgcagcagcaacaacaacaacaacaacagcagcagcagcagctgcagcacacacaactggcagctcacctgggcggcagctactccagcgattcgtacccgatctacgagaatccgtcccgcgtcatctcgatgcgcgccacgcagtcgcagcgatcggagtcgcccatctacagcaatacgacggcctcgtcggccacgctggccgtggttccgcagcatcatcatcagggtcacctggcggtgccatctggaagcgggggaggatccctgagcggcagcggtcgtggtggcagttctggcagtgttcgcggcgcctctacctcagtgcaatcactgtacgtcccaccgcgaactccgcccagtgcggttgccggagcgggaggcagtgccaatgggtcgctgcagaaggtaccatcacagcaatcgctcacggagcccgaggagctgcctctgccgcccggctgggccactcagtacacgctacacggtcggaaatactatattgatcacaatgcgcataccacgcactggaatcatccgttggagcgcgaaggtctgccggtgggctggcggcgggtggtgtccaagatgcatggcacctactatgagaaccagtataccgggcagagccaacgtcagcatccatgcttgacctcctactatgtctacacgacgtctgcggagccaccgaaagcgattcgaccagaggcgtcgctctatgccccacccacgcacactcacaatgcactggtgccggccaatccctatctgctcgaggagatccccaagtggttggccgtctactcggaggcggactcgtccaaggaccacctgctgcagttcaacatgtttagcctgccggagctggagggcttcgacagcatgctggtgcggctcttcaagcaggaactgggcaccatcgtgggcttctacgagcgctaccgtcgcgctttgatactcgagaagaatcgacgcgccggccagaaccagaaccaaaaccagtgacccggtgaccaggtgacgactgactcagaccacatactcgccagcagctatatgcacatcatagtgctcctgtaatcgacctttaacttatttaaccatcgactcatcgcgaaatcagtgccttatacgaaaccagacgagatggtagccaagcagatccatgacagttcgaatgccttgatgaaacgtagaattgtgctacgttctatataaccttaatgtgatttgagcttggcgtttgtttgtaatgtgagcaaagaaaattaaactggtttactgatcatcttaaaaaaaaaaaaaaaaaa

SEQ ID NO: 14 - protein, 230 aa, Drosophila melanogaster
MNYLTILLCNRKPTMLSRRNKEKSQHKEGVVGKYMKKDTPPDISVINVWSDQRAKKKSLQRCASTSPSCEFHPRSSSTSRNTYSCTDSQPDYYHARRAQSQMPLQQHSHSHPHSLPHPSHPHVRSHPPLPPHQFRASSNQLSQNSSNYVNFEQIERMRRQQSSPLLQTTSSPAPGAGGFQRSYSTTQRQHHPHMGGDSYDADQGLLSASYANMLQLPQRPHSPAHYAVPP

SEQ ID NO: 15 - DNA, 2211 bp, Drosophila melanogaster
cgatcgtacagcataaatttagctcgaaaatggactaaacaaagacagcgatccggaatccgagcggaaacatagtctgcatgaactatctaacgatcctgctgtgcaaccgaaaaccgacgatgctctcgcgccggaacaaggagaagtcccagcacaaggagggcgtggtggggaagtacatgaagaaggacaccccaccggatatttcggtgatcaatgtgtggagcgatcagcgggccaagaagaaatcgctgcagcgctgtgcgagcacctcgcccagctgcgagttccatccgcgcagctcgagcaccagtcggaacacctactcctgcacggactcgcagccggactactaccatgctcgacgagcacagagccagatgcccctgcagcagcactcccactcgcatcctcactctctgccccacccctcccatccgcatgtgcgtagtcatcctcccctgccgccccaccagttccgcgccagcagcaatcagttgagtcagaacagcagcaactacgttaatttcgagcagatcgagcggatgcgccgtcagcagtcgtcgccactgctgcagaccacatcatcgccggcgccgggagccggaggattccagcgcagctactccaccacccagcggcagcatcatccccatatgggtggtgacagctacgatgcagatcagggcctgctaagcgcctcctatgccaacatgttgcaactgccccagcggccacactcgcccgctcactacgccgtcccgccgcagcagcagcagcatccacagattcatcaacagcacgcctcgacgccgtttggctccacgctgcggttcgatcgagctgccatgtccatcagggagcgacagcccaggtatcagccaactagctctccgatgcagcagcaacaacaacaacaacaacagcagcagcagcagctgcagcacacacaactggcagctcacctgggcggcagctactccagcgattcgtacccgatctacgagaatccgtcccgcgtcatctcgatgcgcgccacgcagtcgcagcgatcggagtcgcccatctacagcaatacgacggcctcgtcggccacgctggccgtggttccgcagcatcatcatcagggtcacctggcggtgccatctggaagcgggggaggatccctgagcggcagcggtcgtggtggcagttctggcagtgttcgcggcgcctctacctcagtgcaatcactgtacgtcccaccgcgaactccgcccagtgcggttgccggagcgggaggcagtgccaatggtcgctgcagaaggtaccatcacagcaatcgctcacggagcccgaggagctgcctctgccgcccggctgggccactcagtacacgctacacggtcggaaatactatattgatcacaatgcgcataccacgcactggaatcatccgttggagcgcgaaggtctgccggtgggctggcggcgggtggtgtccaagatgcatggcacctactatgagaaccagtataccgggcagagccaacgtcagcatccatgcttgacctcctactatgtctacacgacgtctgcggagccaccgaaagcgattcgaccagaggcgtcgctctatgccccacccacgcacactcacaatgcactggtgccggccaatccctatctgctcgaggagatccccaagtggttggccgtctactcggaggcggactcgtccaaggaccacctgctgcagttcaacatgtttagcctgccggagctggagggcttcgacagcatgctggtgcggctcttcaagcaggaactgggcaccatcgtgggcttctacgagcgctaccgtcgcgctttgatactcgagaagaatcgacgcgccggccagaaccagaaccaaaaccagtgacccggtgaccaggtgacgactgactcagaccacatactcgccagcagctatatgcacatcatagtgctcctgtaatcgacctttaacttatttaaccatcgactcatcgcgaaatcagtgccttatacgaaaccagacgagatggtagccaagcagatccatgacagttcgaatgccttgatgaaacgtagaattgtgctacgttctatataaccttaatgtgatttgagcttggcgtttgtttgtaatgtgagcaaagaaaattaaactggtttactgatcatcttaaaaaaaaaaaaaaaaaa

SEQ ID NO: 16 - protein, 405 aa, Drosophila melanogaster
MNYLTILLCNRKPTMLSRRNKEKSQHKEGVVGKYMKKDTPPDISVINVWSDQRAKKKSLQRCASTSPSCEFHPRSSSTSRNTYSCTDSQPDYYHARRAQSQMPLQQHSHSHPHSLPHPSHPHVRSHPPLPPHQFRASSNQLSQNSSNYVNFEQIERMRRQQSSPLLQTTSSPAPGAGGFQRSYSTTQRQHHPHMGGDSYDADQGLLSASYANMLQLPQRPHSPAHYAVPPQQQQHPQIHQQHASTPFGSTLRFDRAAMSIRERQPRYQPTSSPMQQQQQQQQQQQQQLQHTQLAAHLGGSYSSDSYPIYENPSRVISMRATQSQRSESPIYSNTTASSATLAVVPQHHHQGHLAVPSGSGGGSLSGSGRGGSSGSVRGASTSVQSLYVPPRTPPSAVAGAGGSAN

SEQ ID NO: 17 - DNA, 5720 bp, Drosophila melanogaster
atctagcacgacggcagcaacaaaaccacgaattaattttactaaatttaagccaaacgcgcatcggaaatgcctgaaaatgcgattgaatgcacgcgaaaagtgatgggttgcgaacgcgagtgaatcaagtgaaaatacgtcggcaaatatcagcgaattgtcgtcaaaaggcaaggaaaaacggagaaaaagaggaaaagcaataagtgccgtgtgtgggaaacgcgaaaaaggcgagaacaaagaggcgaaaagcgaggaaattgcgtggaaaacgtggaaaacgcgaagaagcgaagctccaagttggccgccatcgattcgtgcgtaggatcaattaagattccgagtggtcgagaatcggctcaaatcaaattaaaatcaactaatattttggtattcagatattcaaatggaattcattcatcgcctgcgacttttattcggatctgccaactatttttgaatttgaattgtgtgtctgcggctggcgcagaatctctgataaagcagaggaataaaatcggaagaacaacaaatacaaatacaaatgaaatgcggggagcagtatttacatgccaaatgaatgctggataggcgaaagggggggtttctcttataatgcaaatgtgaatgtgaatgcgaatgcgaatgcgagtggaagaattcccggcgcgagtgataaataatccgacgacaaacaaagcagaagcctacaccgcgagaaagagcagcgcaaacacaattatctttattgagagcaacaatatcaagatcgagataataaagcatcctaaaacccgcgccttagttcgttttagtctcgccacggatatagatattcaaaggcaaaaaggtggtgtcggcatcgccagacaaacaagtaaagcatctatttcatacaaaacaaccaattaaataataataaaaataataataatcgtagagaggcagagccaaatcaaattcccggccgccgatgtgccccagtgtgtgtgcgtgtgtgtgtgtgtgtgctgtgctgtgctgtgcgagtgttagtgtgcggagcatttctgtgatatgagtgctaaatgccacagggcgaagcagcagcatcatgcatccagcgggcgaaaaaaggggcggtcgccccaatgataaatacacggcggaagccctcgagagcatcaagcaggacctaacccgatttgaagtacaaaataaccataggaataatcagaattacacacctctgcgatacacggcgaccaacggacgcaacgatgcacttactcctgactatcaccacgccaagcagccgatggagccgccaccctccgcctctcctgctccggacgtggtcataccgccgccgcccgccattgtaggtcagcccggagccggctccatatccgtatccggtgtgggcgttggagtggtgggtgtggcgaacggacgtgtgccaaagatgatgacggccctaatgccaaacaaactgatccggaagccgagcatcgaacgggacacggcgagcagtcactacctgcgctgcagtccggctctggactccggagccggtagctcccgatcggacagcccccattcgcaccacacccaccagccgagctcgaggacggtgggtaatccaggtggaaatggtggattttctccgtcgccaagcggtttcagtgaggtggctccaccggcgccgccgccacgcaatcccaccgcctccagcgcggccacgcccccaccgccagtgccgcccaccagccaggcgtacgtgaagcggcgatcaccggccctgaacaaccgcccgccggcgatagcgccacccactcagcgaggcaactcacctgtaataacccaaaacgggctgaagaacccgcagcagcagttgacgcagcagctgaagtccctgaacctatacccaggcggaggcagtggagcagtggtggagccaccgccgccctacctaattcaaggcggagccggaggagcagcaccgccgccgccaccacccagttacacggcctccatgcagtcgcggcagtcgcccacacaatcccaacaatcggactacaggaaatccccgagcagtgggatatactcggccacctcggcgggctcgccgagccccataactgtgtcgctgccgccggcgccgctggcgaagccacaaccacgagtctaccaggccaggagtcagcagccgatcatcatgcagagtgtgaagagcacgcaggtccaaaagcccgtgctgcaaacagcagtggcgcgccaatcgccatcgagtgcctcggccagcaattcaccagtccacgtgctggccgctccaccctcttaccctcagaagtccgcggcagtggtgcagcagcagcaacaggcagcagcggcggcccaccagcagcagcatcagcaccagcaatccaaaccaccaacgccaaccacaccgcccttggtgggtctgaacagcaagcccaattgcctggagccaccgtcctatgccaagagcatgcaggccaaggcggccacggtggtacagcagcagcaacagcagcagcaacaacagcaggtccagcagcagcaggtgcaacagcagcagcaacagcagcaacagcaactgcaggccttgagggtgctccaggcacaggctcagagggagcgggatcaacgggagcgggaacgggatcagcagaagctggccaacggaaatcctggccggcagatgcttccgccgccgccctatcagagcaacaacaacaacaacagcgagatcaaaccgccgagctgcaacaacaacaacatacagataagcaacagcaacctggcgacgacaccacccattccgcctgccaaatacaataacaactcctccaacacgggcgcgaatagctcgggcggcagcaacggatccaccggcaccaccgcctcctcgtcgaccagctgcaagaagatcaagcacgcctcgcccatcccggagcgcaagaagatctccaaggagaaggaggaggagcgcaaggagttccgcatcaggcagtactcgccgcaagccttcaagttcttcatggagcagcacatagagaacgtgatcaagtcgtatcgccagcgcacgtatcgcaagaatcagctggagaaggagatgcacaaagtgggactgcccgatcagacccaaatcgagatgaggaaaatgctgaaccaaaaggagagcaactacattcgattgaagcgcgccaagatggacaagagcatgttcgtcaaactgaagcccattggagtgggtgcatttggcgaggtaacgctggtgagcaaaatcgatacctcgaaccatttgtatgcgatgaaaaccctgcggaaagcggacgttctcaagcggaatcaggtggcacacgtgaaggccgagagggatatcctcgcggaagccgacaataactgggtggtgaagttgtactacagcttccaggacaaggataatctgtactttgtgatggactacataccaggtggtgatctgatgtcgctgctcatcaaactgggcattttcgaggaggaactggccagattctacatcgccgaggtcacctgcgccgtggacagcgttcacaaaatgggcttcattcacagagacatcaagcctgacaacatactcatcgatagggacggacacataaagctcaccgactttggcctgtgcacgggattccgatggacgcacaactcgaagtactaccaggagaacggcaatcactcgcgccaggactcgatggagccctgggaggaatactccgagaacggaccgaagcccaccgtgctggagaggcgacggatgcgcgatcaccaaagagtcctggcccactcgctggtgggcaccccgaactacatagctcccgaggtgctggagaggagtgggtacacgcagctgtgcgactactggagcgtgggcgtcatcctttacgagatgctggtgggtcagccgccctttctggccaacagtccgctggaaacgcaacaaaaggtcatcaactgggagaaaacgctgcatattccgccgcaggccgagttatcccgcgaggctacggacttgataaggaggctctgtgcgtcggctgacaagcggctgggcaagagcgtggacgaggtcaagagccacgacttcttcaagggcatcgactttgcggacatgcggaagcagaaagcgccctacataccggaaatcaagcacccaacggacacatccaactttgatcccgtggatccggagaagctgcgctcgaatgactccaccatgagcagcggcgatgatgtcgaccagaatgaccgcactttccacggctttttcgaatttaccttccgtcgcttcttcgacgacaagcagccgccggatatgacggacgatcaggcgccggtttacgtctgaaatggatgctctccatgtgcccaacaccaacaccccgcccccgaatcattgttagtcaaatagtcacaaaaaggggatagaaaccattgagtgggcttgcattgtaaaggaagcgtggctatagaatgaaactatctatatacattatataaattataggagacagtagaggcgggagctacgtatatacatacaaataatatacatatatttgatatatatatatatatatatgccgtagggcatgaactgaataaatataaaacggagccgagtagagatgaaacgagaggagcgagtcaggaccttcgacctttaactgaacatagtatatccttgtgcactactactccacaacaaatatatatttttaaattgttagaattcaaaagggaccaactggaaatcgaacctttctggtgctcaaagcaaagcaaagcaaagcaaaacaaaacgccttaaactaaatgagacgcgaatttacccaaccacttcactcctctcctttctccacctccgatcggtggccggattcgaactcagcaggctggttgcatccggccatcccattgacttcccattcagaattgagattgcgaggtgtgcgatggagaacgaacggagaccaaaagtcgcacggcagcgatataagcgggtcttataagcctaatctaaatctaaactgggagaacaggacctatgtatgtcctgctatccaattcgtctatcactgctcttcatctgtgtacgacccccaccccccccctccccatccaaaagaacaaacttagacgtagcctatgtgaaaagctagcaatgttagaccaacttgttgaatgccaaatgaaattgtttagccccacgaggaaaacgcgggggaaattcaacacttattctctgatagcaaacggaaaagaaagaaagaaaaaaaaaaacagaaacagtacgagaaaattgtaatcttcttaatgtaatattgtaaagaacacgttaattgtaatctatgctagagttgtgtagcgccctaagatgttttttagtttatagaccgctaaccgtaatctagtttaattcctaacactaagcgagagtacagtacattggtttttttgtttgtcgtaggttcgttggaaaatgcttaacgggaaacgatttgtttttctctttaattagcttcagtttgtatgtgcgtgtgtttttattatgacttatatatagtccatctgaatattcgtggatggagcctattttaaatgtgagatcgagctaattgaaggaaatacaaacaaactctgtgtgccttggccaattagtttac

SEQ ID NO: 18 - protein, 1099 aa, Drosophila melanogaster
MHPAGEKRGGRPNDKYTAEALESIKQDLTRFEVQNNHRNNQNYTPLRYTATNGRNDALTPDYHHAKQPMEPPPSASPAPDVVIPPPPAIVGQPGAGSISVSGVGVGVVGVANGRVPKMMTALMPNKLIRKPSIERDTASSHYLRCSPALDSGAGSSRSDSPHSHHTHQPSSRTVGNPGGNGGFSPSPSGFSEVAPPAPPPRNPTASSAATPPPPVPPTSQAYVKRRSPALNNRPPAIAPPTQRGNSPVITQNGLKNPQQQLTQQLKSLNLYPGGGSGAVVEPPPPYLIQGGAGGAAPPPPPPSYTASMQSRQSPTQSQQSDYRKSPSSGIYSATSAGSPSPITVSLPPAPLAKPQPRVYQARSQQPIIMQSVKSTQVQKPVLQTAVARQSPSSASASNSPVHVLAAPPSYPQKSAAVVQQQQQAAAAAHQQQHQHQQSKPPTPTTPPLVGLNSKPNCLEPPSYAKSMQAKAATVVQQQQQQQQQQQVQQQQVQQQQQQQQQQLQALRVLQAQAQRERDQRERERDQQKLANGNPGRQMLPPPPYQSNNNNNSEIKPPSCNNNNIQISNSNLATTPPIPPAKYNNNSSNTGANSSGGSNGSTGTTASSSTSCKKIKHASPIPERKKISKEKEEERKEFRIRQYSPQAFKFFMEQHIENVIKSYRQRTYRKNQLEKEMHKVGLPDQTQIEMRKMLNQKESNYIRLKRAKMDKSMFVKLKPIGVGAFGEVTLVSKIDTSNHLYAMKTLRKADVLKRNQVAHVKAERDILAEADNNWVVKLYYSFQDKDNLYFVMDYIPGGDLMSLLIKLGIFEEELARFYIAEVTCAVDSVHKMGFIHRDIKPDNILIDRDGHIKLTDFGLCTGFRWTHNSKYYQENGNHSRQDSMEPWEEYSENGPKPTVLERRRMRDHQRVLAHSLVGTPNYIAPEVLERSGYTQLCDYWSVGVILYEMLVGQPPFLANSPLETQQKVINWEKTLHIPPQAELSREATDLIRRLCASADKRLGKSVDEVKSHDFFKGIDFADMRKQKAPYIPEIKHPTDTSNFDPVDPEKLRSNDSTMSSGDDVDQNDRTFHGFFEFTFRRFFDDKQPPDMTDDQAPVYV

SEQ ID NO: 19 - DNA, 5360 bp, Drosophila melanogaster
gtctatcgatatcggtctatcgatatgtgaacatatctagcacgacggcagcaacaaaaccacgaattaattttactaaatttaagccaaacgcgcatcggaaatgcctgaaaatgcgattgaatgcacgcgaaaagtgatgggttgcgaacgcgagtgaatcaagtgaaaatacgtcggcaaatatcagcgaattgtcgtcaaaaggcaaggaaaaacggagaaaaagaggaaaagcaataagtgccgtgtgtgggaaacgcgaaaaaggcgagaacaaagaggcgaaaagcgaggaaattgcgtggaaaacgtggaaaacgcgaagaagcgaagctccaagttggccgccatcgattcgtgtgtaggatcaattaagattccgagtggtcgagaatcggctcaaatcaaattaaaatcaactaatattttggtattcagatattcaaatggaattcattcatcgcctgcgacttttattcggatctgccaactatttttgaatttgaattgtgtgtctgcggctggcgcagaatctctgataaagcagaggaataaaatcggaagaacaacaaatacaaatacaaatgaaatgcggggagcagtatttacatgccaaatgaatgctggataggcgaaagggggggggtttctcttataatgcaaatgtgaatgtgaatgcgaatgcgaatgcgagtggaagaattcccggcgcgagtgataaataatccgacgacaaacaaagcagaagcctacaccgcgagaaagagcagtgcaaacacaattatctttattgagagcaacaatatcaagatcgagataataaagcatcctaaaacccgcgccttagttcgttttagtctcgccacggatatagatattcaaaggcaaaaaggtggtgtcggcatcgccagacaaacaagtaaagcatctatttcatacaaaacaaccaattaaataataataaaaataataataatcgtagagaggcagagccaaatcaaattcccggccgccgatgtgccccagtgtgtgtgcgtgtgtgtgtgtgtgctgtgctgtgctgtgcgagtgttagtgtgcggagcatttctgtgatatgagtgctaaatgccacagggcgaagcagcagcatcatgcatccagcgggcgaaaaaaggggcggtcgccccaatgataaatacacggcggaagccctcgagagcatcaagcaggacctaacccgatttgaagtacaaaataaccataggaataatcagaattacacacctctgagatacacggcgaccaacggacgcaacgatgcacttactcctgactatcaccacgccaagcagccgatggagccgccaccctccgcctctcctgctccggacgtggtcataccgccgccgcccgccattgtaggtcagcccggagccggctccatatccgtatccggtgtgggcgttggagtggtgggtgtggcgaacggacgtgtgccaaagatgatgacggccctaatgccaaacaaactgatccggaagccgagcatcgaacgggacacggcgagcagtcactacctgcgctgcagtccggctctggactccggagccggtagctcccgatcggacagcccccattcgcaccacacccaccagccgagctcgaggacggtgggtaatccaggtggaaatggtggattttctccgtcgccaagcggtttcagtgaggtggctccaccggcgccgccgccacgcaatcccaccgcctccagcgcggccacgcccccaccgccagtgccgcccaccagccaggcgtacgtgaagcggcgatcaccggccctgaacaaccgcccgccggcgatagcgccacccactcagcgaggcaactcacctgtaataacccaaaacgggctgaagaacccgcagcagcagttgacgcagcagctgaagtccctgaacctatacccaggcggaggcagtggagcagtggtggagccaccgccgccctacctaattcaaggcggagccggaggagcagcaccgccgccgccaccacccagttacacggcctccatgcagtcgcggcagtcgcccacacaatcccaacaatcggactacaggaaatccccgagcagtgggatatactcggccacctcggcgggctcgccgagccccataactgtgtccctgccgccggcgccgctggcgaagccacaaccacgagtctaccaggccaggagtcagcagccgatcatcatgcagagtgtgaagagcacgcaggtccaaaagcccgtgctgcaaacagcagtggcgccccaatcgccatcgagtgcctcggccagcaattcaccagtccacgtgctggccgctccaccctcttaccctcagaagtccgcggcagtggtgcagcagcagcaacaggcagcagcggcggcccaccagcagcagcatcagcaccagcaatccaaaccagcaacgcccaccacaccgcccttggtgggtctgaacagcaagcccaattgcctggagccaccgtcctatgccaagagcatgcaggccaaggcggccacggtggtacagcagcagcaacagcagcagcaacaacagcaggtccagcagcagcaggtgcaacagcagcagcaacagcagcaacagcaactgcaggccttgagggtgctccaggcacaggctcagagggagcgggatcaacgggagcgggaacgggatcagcagaagctggccaacggaaatcctggccggcagatgcttccgccgccgccctatcagagcaacaacaacaacaacagcgagatcaaaccgccgagctgcaacaacaacaacatacagataagcaacagcaacctggcgacgacaccacccattccgcctgccaaatacaataacaactcctccaacacgggcgcgaatagctcgggcggcagcaacggatccaccggcaccaccgcctcctcgtcgaccagctgcaagaagatcaagcacgcctcgcccatcccggagcgcaagaagatctccaaggagaaggaggaggagcgcaaggagttccgcatcaggcagtactcgccgcaagccttcaagttcttcatggagcagcacatagagaacgtgatcaagtcgtatcgccagcgcacgtatcgcaagaatcagctggagaaggagatgcacaaagtgggactgcccgatcagacccaaatcgagatgaggaaaatgctgaaccaaaaggagagcaactacattcgattgaagcgcgccaagatggacaagagcatgttcgtcaaactgaagcccattggagtgggtgcatttggcgaggtaacgctggtgagcaaaatcgatacctcgaaccatttgtatgcgatgaaaaccctgcggaaagcggacgttctcaagcggaatcaggtggcacacgtgaaggccgagagggatatcctcgcggaagccgacaataactgggtggtgaagttgtactacagcttccaggacaaggataatctgtactttgtgatggactacataccaggtggtgatctgatgtcgctgctcatcaaactgggcattttcgaggaggaactggccagattctacatcgccgaggtcacctgcgccgtggacagcgttcacaaaatgggcttcattcacagagacatcaagcctgacaacatactcatcgatagggacggacacataaagctcaccgactttggcctttgcacgggattccgatggacgcacaactcgaagtactaccaggagaacggcaatcactcgcgccaggactcgatggagccctgggaggaatactccgagaacggacccaagcccaccgtgctggagaggcgacggatgcgcgatcaccaaagagtcctggcccactcgctggtgggcaccccgaactacatagctcccgaggtgctggagaggagtgggtacacgcagctgtgcgactactggagcgtgggcgtcatcctttacgagatgctggtgggtcagccgccctttctggccaacagtccgctggaaacgcaacaaaaggtcatcaactgggagaaaacgctgcatattccgccgcaggccgagttatcccgcgaggctacggacttgataaggaggctctgtgcgtcggctgacaagcggctgggcaagagcgtggacgaggtcaagagccacgacttcttcaagggcatcgactttgcggacatgcggaagcagaaagcgccctacataccggaaatcaagcacccaacggacacatccaactttgatcccgtggatccggagaagctgcgctcgaatgactccaccatgagcagcggcgatgatgtcgaccagaatgaccgcactttccacggctttttcgaatttaccttccgtcgcttcttcgacgacaagcagccgccggatatgacggacgatcaggcgccggtttacgtctgaaatggatgctctccatgtgcccaacaccaacaccccgcccccgaatcattgttagtcaaatagtcacaaaaaggggatagaaaccattgagtgggcttgcattgtaaaggaagcgtggctatagaatgaaactatctatatacattatataaattataggagacagtagaggcgggagctacgtatatacatacaaataatatacatatatttgatatatatatatatatatatgccgtagggcatgaactgaataaatataaaacggagccgagtagagatgaaacgagaggagcgagtcaggaccttcgacctttaactgaacatagtatatccttgtgcactactactctacaacaaatatatatttttaaattgttagaattcaaaagggaccaactggaaatcgaacctttctggtgctcaaagcaaagcaaagcaaagcaaaacaaaacgccttaaactaaacgagacgcgaatttacccaaccacttcactcctctcctttctccacctccgatcggtggccggattcgaactcagcaggctggttgcatccggccatcccattgacttctcattcagaattgagattgcgaggtgtgcgatggagaacgaacggagaccaaaagtcgcacggcagcgatataagcgggtcttataagcctaatctaaatctaaactgggagaacaggacctatgtatgtgttattagtgtcctgctatccaattcgtctatcactgctcttcatctgtgtacccccctcccccaccacccctccccatccaaaagaacaaacttagacgtagcctatgtgaaaagctagcaatgttagaccaacttgttgaatgccaaatgaaattgtttagccccgcgaggaaaacgcggaggaaattcaacacttattctctgatagcaaccgg

SEQ ID NO: 20 - protein, 1099 aa, Drosophila melanogaster
MHPAGEKRGGRPNDKYTAEALESIKQDLTRFEVQNNHRNNQNYTPLRYTATNGRNDALTPDYHHAKQPMEPPPSASPAPDVVIPPPPAIVGQPGAGSISVSGVGVGVVGVANGRVPKMMTALMPNKLIRKPSIERDTASSHYLRCSPALDSGAGSSRSDSPHSHHTHQPSSRTVGNPGGNGGFSPSPSGFSEVAPPAPPPRNPTASSAATPPPPVPPTSQAYVKRRSPALNNRPPAIAPPTQRGNSPVITQNGLKNPQQQLTQQLKSLNLYPGGGSGAVVEPPPPYLIQGGAGGAAPPPPPPSYTASMQSRQSPTQSQQSDYRKSPSSGIYSATSAGSPSPITVSLPPAPLAKPQPRVYQARSQQPIIMQSVKSTQVQKPVLQTAVAPQSPSSASASNSPVHVLAAPPSYPQKSAAVVQQQQQAAAAAHQQQHQHQQSKPATPTTPPLVGLNSKPNCLEPPSYAKSMQAKAATVVQQQQQQQQQQQVQQQQVQQQQQQQQQQLQALRVLQAQAQRERDQRERERDQQKLANGNPGRQMLPPPPYQSNNNNNSEIKPPSCNNNNIQISNSNLATTPPIPPAKYNNNSSNTGANSSGGSNGSTGTTASSSTSCKKIKHASPIPERKKISKEKEEERKEFRIRQYSPQAFKFFMEQHIENVIKSYRQRTYRKNQLEKEMHKVGLPDQTQIEMRKMLNQKESNYIRLKRAKMDKSMFVKLKPIGVGAFGEVTLVSKIDTSNHLYAMKTLRKADVLKRNQVAHVKAERDILAEADNNWVVKLYYSFQDKDNLYFVMDYIPGGDLMSLLIKLGIFEEELARFYIAEVTCAVDSVHKMGFIHRDIKPDNILIDRDGHIKLTDFGLCTGFRWTHNSKYYQENGNHSRQDSMEPWEEYSENGPKPTVLERRRMRDHQRVLAHSLVGTPNYIAPEVLERSGYTQLCDYWSVGVILYEMLVGQPPFLANSPLETQQKVINWEKTLHIPPQAELSREATDLIRRLCASADKRLGKSVDEVKSHDFFKGIDFADMRKQKAPYIPEIKHPTDTSNFDPVDPEKLRSNDSTMSSGDDVDQNDRTFHGFFEFTFRRFFDDKQPPDMTDDQAPVYV

SEQ ID NO: 21 - DNA, 4307 bp, Homo sapiens
gcgacgctcacgaacgatcagagctgcgggcgacgcaacgaagcccggaggccgcaggctgcgcgctccctcgcagcagccgggcgggcaaaagcccccagtcctcggcccccgcgcaagcgacgccgggaaatgcccacatccgggaaacctgcagcggagtgcggcggcggcgacactgagtggaaggcaaaatggcggcggcggcggcggtggcctggtgttaaggggagagccaggtccttacgacccctgggacgggccgcgctggcccgcggcagcccccccgttcgtctccccgctctgccccaccagggatacttggggttgctgggacggactctggccgcctcagcgtccgccctcaggcccgtggccgctgtccaggagctctgctctcccctccagagttaattatttatattgtaaagaattttaacagtcctggggacttccttgaaggatcattttcacttttgctcagaagaaagctctggatctatcaaataaagaagtccttcgtgtgggctacatatatagatgttttcatgaagaggagtgaaaagccagaaggatatagacaaatgaggcctaagacctttcctgccagtaactatactgtcagtagccggcaaatgttacaagaaattcgggaatcccttaggaatttatctaaaccatctgatgctgctaaggctgagcataacatgagtaaaatgtcaaccgaagatcctcgacaagtcagaaatccacccaaatttgggacgcatcataaagccttgcaggaaattcgaaactctctgcttccatttgcaaatgaaacaaattcttctcggagtacttcagaagttaatccacaaatgcttcaagacttgcaagctgctggatttgatgaggatatggttatacaagctcttcagaaaactaacaacagaagtatagaagcagcaattgaattcattagtaaaatgagttaccaagatcctcgacgagagcagatggctgcagcagctgccagacctattaatgccagcatgaaaccagggaatgtgcagcaatcagttaaccgcaaacagagctggaaaggttctaaagaatccttagttcctcagaggcatggcccgccactaggagaaagtgtggcctatcattctgagagtcccaactcacagacagatgtaggaagacctttgtctggatctggtatatcagcatttgttcaagctcaccctagcaacggacagagagtgaaccccccaccaccacctcaagtaaggagtgttactcctccaccacctccaagaggccagactccccctccaagaggtacaactccacctcccccttcatgggaaccaaactctcaaacaaagcgctattctggaaacatggaatacgtaatctcccgaatctctcctgtcccacctggggcatggcaagagggctatcctccaccacctctcaacacttcccccatgaatcctcctaatcaaggacagagaggcattagttctgttcctgttggcagacaaccaatcatcatgcagagttctagcaaatttaactttccatcagggagacctggaatgcagaatggtactggacaaactgatttcatgatacaccaaaatgttgtccctgctggcactgtgaatcggcagccaccacctccatatcctctgacagcagctaatggacaaagcccttctgctttacaaacagggggatctgctgctccttcgtcatatacaaatggaagtattcctcagtctatgatggtgccaaacagaaatagtcataacatggaactatataacattagtgtacctggactgcaaacaaattggcctcagtcatcttctgctccagcccagtcatccccgagcagtgggcatgaaatccctacatggcaacctaacataccagtgaggtcaaattcttttaataacccattaggaaatagagcaagtcactctgctaattctcagccttctgctacaacagtcactgcaattacaccagctcctattcaacagcctgtgaaaagtatgcgtgtattaaaaccagagctacagactgctttagcacctacacacccttcttggataccacagccaattcaaactgttcaacccagtccttttcctgagggaaccgcttcaaatgtgactgtgatgccacctgttgctgaagctccaaactatcaaggaccaccaccaccctacccaaaacatctgctgcaccaaaacccatctgttcctccatacgagtcaatcagtaagcctagcaaagaggatcagccaagcttgcccaaggaagatgagagtgaaaagagttatgaaaatgttgatagtggggataaagaaaagaaacagattacaacttcacctattactgttaggaaaaacaagaaagatgaagagcgaagggaatctcgtattcaaagttattctcctcaagcatttaaattctttatggagcaacatgtagaaaatgtactcaaatctcatcagcagcgtctacatcgtaaaaaacaattagagaatgaaatgatgcgggttggattatctcaagatgcccaggatcaaatgagaaagatgctttgccaaaaagaatctaattacatccgtcttaaaagggctaaaatggacaagtctatgtttgtgaagataaagacactaggaataggagcatttggtgaagtctgtctagcaagaaaagtagatactaaggctttgtatgcaacaaaaactcttcgaaagaaagatgttcttcttcgaaatcaagtcgctcatgttaaggctgagagagatatcctggctgaagctgacaatgaatgggtagttcgtctatattattcattccaagataaggacaatttatactttgtaatggactacattcctgggggtgatatgatgagcctattaattagaatgggcatctttccagaaagtctggcacgattctacatagcagaacttacctgtgcagttgaaagtgttcataaaatgggttttattcatagagatattaaacctgataatattttgattgatcgtgatggtcatattaaattgactgactttggcctctgcactggcttcagatggacacacgattctaagtactatcagagtggtgaccatccacggcaagatagcatggatttcagtaatgaatggggggatccctcaagctgtcgatgtggagacagactgaagccattagagcggagagctgcacgccagcaccagcgatgtctagcacattctttggttgggactcccaattatattgcacctgaagtgttgctacgaacaggatacacacagttgtgtgattggtggagtgttggtgttattctttttgaaatgttggtgggacaacctcctttcttggcacaaacaccattagaaacacaaatgaaggttatcaactggcaaacatctcttcacattccaccacaagctaaactcagtcctgaagcttctgatcttattattaaactttgccgaggacccgaagatcgcttaggcaagaatggtgctgatgaaataaaagctcatccattttttaaaacaattgacttctccagtgacctgagacagcagtctgcttcatacattcctaaaatcacacacccaacagatacatcaaattttgatcctgttgatcctgataaattatggagtgatgataacgaggaagaaaatgtaaatgacactctcaatggatggtataaaaatggaaagcatcctgaacatgcattctatgaatttaccttccgaaggttttttgatgacaatggctacccatataattatccgaagcctattgaatatgaatacattaattcacaaggctcagagcagcagtcggatgaagatgatcaaaacacaggctcagagattaaaaatcgcgatctagtatatgtttaacacactagtaaataaatgtaatgaggatttgtaaaagggcctgaaatgcgaggtgttttgaggttctgagagtaaaattatgcaaatatgacagagctatatatgtgtgctctgtgtacaatattttattttcctaaattatgggaaatccttttaaaatgttaatttattccagccgtttaaatcagtatttagaaaaaaattgttataaggaaagtaaattatgaactgaatattatagtcagttcttggtacttaaagtacttaaaataagtagtgctttgtttaaaaggagaaacctggtatctatttgtatatatgctaaataattttaaaatacaagagtttttgaaatttttttctaga

SEQ ID NO: 22 - protein, 1130 aa, Homo sapiens
MKRSEKPEGYRQMRPKTFPASNYTVSSRQMLQEIRESLRNLSKPSDAAKAEHNMSKMSTEDPRQVRNPPKFGTHHKALQEIRNSLLPFANETNSSRSTSEVNPQMLQDLQAAGFDEDMVIQALQKTNNRSIEAAIEFISKMSYQDPRREQMAAAAARPINASMKPGNVQQSVNRKQSWKGSKESLVPQRHGPPLGESVAYHSESPNSQTDVGRPLSGSGISAFVQAHPSNGQRVNPPPPPQVRSVTPPPPPRGQTPPPRGTTPPPPSWEPNSQTKRYSGNMEYVISRISPVPPGAWQEGYPPPPLNTSPMNPPNQGQRGISSVPVGRQPIIMQSSSKFNFPSGRPGMQNGTGQTDFMIHQNVVPAGTVNRQPPPPYPLTAANGQSPSALQTGGSAAPSSYTNGSIPQSMMVPNRNSHNMELYNISVPGLQTNWPQSSSAPAQSSPSSGHEIPTWQPNIPVRSNSFNNPLGNRASHSANSQPSATTVTAITPAPIQQPVKSMRVLKPELQTALAPTHPSWIPQPIQTVQPSPFPEGTASNVTVMPPVAEAPNYQGPPPPYPKHLLHQNPSVPPYESISKPSKEDQPSLPKEDESEKSYENVDSGDKEKKQITTSPITVRKNKKDEERRESRIQSYSPQAFKFFMEQHVENVLKSHQQRLHRKKQLENEMMRVGLSQDAQDQMRKMLCQKESNYIRLKRAKMDKSMFVKIKTLGIGAFGEVCLARKVDTKALYATKTLRKKDVLLRNQVAHVKAERDILAEADNEWVVRLYYSFQDKDNLYFVMDYIPGGDMMSLLIRMGIFPESLARFYIAELTCAVESVHKMGFIHRDIKPDNILIDRDGHIKLTDFGLCTGFRWTHDSKYYQSGDHPRQDSMDFSNEWGDPSSCRCGDRLKPLERRAARQHQRCLAHSLVGTPNYIAPEVLLRTGYTQLCDWWSVGVILFEMLVGQPPFLAQTPLETQMKVINWQTSLHIPPQAKLSPEASDLIIKLCRGPEDRLGKNGADEIKAHPFFKTIDFSSDLRQQSASYIPKITHPTDTSNFDPVDPDKLWSDDNEEENVNDTLNGWYKNGKHPEHAFYEFTFRRFFDDNGYPYNYPKPIEYEYINSQGSEQQSDEDDQNTGSEIKNRDLVYV

SEQ ID NO: 23 - DNA, 3412 bp, Homo sapiens
caaacagtgacacttccctggatgccaaagtcctggggagcaaagatgccaccagcagcaagcagcagatgagagccaccccaaagttcggaccttatcagaaagccttgagggaaatcagatattccttgttgccttttgctaatgaatcgggcacctctgcagctgcagaagtgaaccggcaaatgctgcaggaactggtgaacgcaggatgcgaccaggagatggctggccgagctctcaagcagactggcagcaggagcatcgaggccgccctggagtacatcagcaagatgggctacctggacccgaggaatgagcagattgtgcgggtcattaagcagacctccccaggaaaggggctcatgccaaccccagtgacgcggaggcccagcttcgaaggaaccggcgattcgtttgcgtcctaccaccagctgagcggtaccccctacgagggcccaagcttcggcgctgacggccccacggcgctggaggagatgccgcggccgtacgtggactaccttttccccggagtcggcccccacgggcccggccaccagcaccagcacccacccaagggctacggtgccagcgtagaggcagcaggggcacacttcccgctgcagggcgcgcactacgggcggccgcacctgctggtgcctggggaacccctgggctacggagtgcagcgcagcccctccttccagagcaagacgccgccggagaccgggggttacgccagcctgcccacgaagggccagggaggaccgccaggcgccggcctcgctttcccaccccctgccgccgggctctacgtgccgcacccacaccacaagcaggccggtcccgtggcccaccagctgcatgtgctgggctcccgcagccaggtgttcgccagcgacagccccccgcagagcctgctcactccctcgcggaacagcctcaacgtggacctgtatgaattgagcagcacctccgtccagcagtggccggctgccaccctggcccgccgggactccctgcagaagccgggcctggaggcgccgccgcgcgcgcacgtggccttccggcctgactgcccagtgcccagcaggaccaactccttcaacagccaccagccgcggcccggtccgcctggcaaggccgagccctccctgcccgcccccaacaccgtgacggctgtcagcgccgcgcacatcttgcacccggtgaagagcgtgcgtgtgctgaggccggagccgcagacggctgtggggccctcgcaccccgcctgggtgcccgcgcctgccccggcccccgcccccgcccccgccccggctgcggagggcttggacgccaaggaggagcatgccctggcgctgggcggcgcaggcgccttcccgctggacgtggagtacggaggcccagaccggagtgcccgcctccgccctacccgaagcacctgctgctgcgcaaagtcggagcagtacgacctggacagcctgtgcgcaggcatggagcagagcctccgtgcgggccccaacgagcccgagggcggcgacaagagccgcaaaagcgccaagggggacaaaggcggaaaggataaaaagcagattcagacctctcccgttcccgtccgcaaaaacagcagagacgaagagaagagagagtcacgcatcaagagctactcgccatacgcctttaagttcttcatggagcagcacgtggagaatgtcatcaaaacctaccagcagaaggttaaccggaggctgcagctggagcaagaaatggccaaagctggactctgtgaagctgagcaggagcagatgcggaagatcctctaccagaaagagtctaattacaacaggttaaagagggccaagatggacaagtctatgtttgtcaagatcaaaaccctggggatcggtgcctttggagaagtgtgccttgcttgtaaggtggacactcacgccctgtacgccatgaagaccctaaggaaaaaggatgtcctgaaccggaatcaggtggcccacgtcaaggccgagagggacatcctggccgaggcagacaatgagtgggtggtcaaactctactactccttccaagacaaagacagcctgtactttgtgatggactacatccctggtggggacatgatgagcctgctgatccggatggaggtcttccctgagcacctggcccggttctacatcgcagagctgactttggccattgagagtgtccacaagatgggcttcatccaccgagacatcaagcctgataacattttgatagatctggatggtcacattaaactcacagatttcggcctctgcactgggttcaggtggactcacaattccaaatattaccagaaagggagccatgtcagacaggacagcatggagcccagcgacctctgggatgatgtgtctaactgtcggtgtggggacaggctgaagaccctagagcagagggcgcggaagcagcaccagaggtcgctggcacattcactggtggggactccaaactacatcgcacccgaggtgctcctccgcaaagggtacactcaactctgtgactggtggagtgttggagtgattctcttcgagatgctggtggggcagccgccctttttggcacctactcccacagaaacccagctgaaggtgatcaactgggagaacacgctccacattccagcccaggtgaagctgagccctgaggccagggacctcatcaccaagctgtgctgctccgcagaccaccgcctggggcggaatggggccgatgacctgaaggcccaccccttcttcagcgccattgacttctccagtgacatccggaagcagccagccccctacgttcccaccatcagccaccccatggacacctcgaatttcgaccccgtagatgaagaaagcccttggaacgatgccagcgaaggtagcaccaaggcctgggacacactcacctcgcccaataacaagcatcctgagcacgcattttacgaattcaccttccgaaggttctttgatgacaatggctacccctttcgatgcccaaagccttcaggagcagaagcttcacaggctgagagctcagatttagaaagctctgatctggtggatcagactgaaggctgccagcctgtgtacgtgtagatgggggccaggcacccccaccactcgctgcctcccaggtcagggtcccggagccggtgccctcacaggccaatagggaagccgagggctgttttgttttaaattagtccgtcgattacttcacttgaaattctgctcttcaccaagaaaacccaaacaggacacttttgaaaacaggactcagcatcgctttcaataggcttttcaggaccttcactgcattaaaacaatatttttgaaaaatttaaaaaaaaaaaaaaaaaaaaaaa

SEQ ID NO: 24 - protein, 1046 aa, Homo sapiens
NSDTSLDAKVLGSKDATSSKQQMRATPKFGPYQKALREIRYSLLPFANESGTSAAAEVNRQMLQELVNAGCDQEMAGRALKQTGSRSIEAALEYISKMGYLDPRNEQIVRVIKQTSPGKGLMPTPVTRRPSFEGTGDSFASYHQLSGTPYEGPSFGADGPTALEEMPRPYVDYLFPGVGPHGPGHQHQHPPKGYGASVEAAGAHFPLQGAHYGRPHLLVPGEPLGYGVQRSPSFQSKTPPETGGYASLPTKGQGGPPGAGLAFPPPAAGLYVPHPHHKQAGPVAHQLHVLGSRSQVFASDSPPQSLLTPSRNSLNVDLYELSSTSVQQWPAATLARRDSLQKPGLEAPPRAHVAFRPDCPVPSRTNSFNSHQPRPGPPGKAEPSLPAPNTVTAVSAAHILHPVKSVRVLRPEPQTAVGPSHPAWVPAPAPAPAPAPAPAAEGLDAKEEHALALGGAGAFPLDVEYGGPDRSARLRPTRSTCCCAKSEQYDLDSLCAGMEQSLRAGPNEPEGGDKSRKSAKGDKGGKDKKQIQTSPVPVRKNSRDEEKRESRIKSYSPYAFKFFMEQHVENVIKTYQQKVNRRLQLEQEMAKAGLCEAEQEQMRKILYQKESNYNRLKRAKMDKSMFVKIKTLGIGAFGEVCLACKVDTHALYAMKTLRKKDVLNRNQVAHVKAERDILAEADNEWVVKLYYSFQDKDSLYFVMDYIPGGDMMSLLIRMEVFPEHLARFYIAELTLAIESVHKMGFIHRDIKPDNILIDLDGHIKLTDFGLCTGFRWTHNSKYYQKGSHVRQDSMEPSDLWDDVSNCRCGDRLKTLEQRARKQHQRSLAHSLVGTPNYIAPEVLLRKGYTQLCDWWSVGVILFEMLVGQPPFLAPTPTETQLKVINWENTLHIPAQVKLSPEARDLITKLCCSADHRLGRNGADDLKAHPFFSAIDFSSDIRKQPAPYVPTISHPMDTSNFDPVDEESPWNDASEGSTKAWDTLTSPNNKHPEHAFYEFTFRRFFDDNGYPFRCPKPSGAEASQAESSDLESSDLVDQTEGCQPVYV

SEQ ID NO: 25 - protein, 17 aa, Artificial Sequence
  Synthetic Sequence
PYGRQMLPPPPYQSNNN

SEQ ID NO: 26 - protein, 17 aa, Artificial Sequence
  Synthetic Sequence
PAGRQMLPPPPAQSNNN